(19) United States
(12) Patent Application Publication
(10) Pub. No.: US 2026/0033816 A1
(43) Pub. Date: Feb. 5, 2026

(54) DEVICES AND METHODS FOR ANCHORING A SLEEVE IN A TISSUE CAVITY

(71) Applicant: AVERTO MEDICAL, INC., Fremont, CA (US)

(72) Inventors: Kenton D. FONG, Belmont, CA (US); Jeffrey W. ETTER, Hayward, CA (US); Dean HU, San Leandro, CA (US)

(21) Appl. No.: 18/986,319

(22) Filed: Dec. 18, 2024

Related U.S. Application Data
(60) Provisional application No. 63/678,843, filed on Aug. 2, 2024.
(60) Provisional application No. 63/728,531, filed on Dec. 5, 2024.

(51) Int. Cl. A61B 17/00

(52) U.S. Cl. CPC A61B 17/00234; A61B 2017/00238; A61B 2017/00296; A61B 2017/00398; A61B 2017/00544; A61B 2017/00557; A61B 2017/00566; A61B 2017/00734; A61B 2017/00845

(57) ABSTRACT

According to embodiments of the invention, an anchoring system includes a sleeve having an inner surface defining a lumen, a first expandable sealing mechanism disposed along a proximal end of the sleeve, and a second expandable sealing mechanism disposed along the proximal end of the sleeve. The anchoring system further includes roughening material disposed on an outer surface of the sleeve. Expansion of the first and second expandable sealing mechanisms and application of negative pressure to the anchoring system, causes a seal to form between the first and second expandable sealing mechanisms, the outer surface of the sleeve, and an inner surface of a tissue cavity. According to some embodiments, a sleeve body is included with the sleeve, with the first expandable sealing mechanism being disposed at a proximal end of the sleeve body and the second expandable sealing mechanism being disposed at a distal end of the sleeve body.

### CROSS-REFERENCE TO RELATED APPLICATIONS

This application claims the benefit of priority from U.S. Provisional Application No. 63/728,531, filed Dec. 5, 2024, and U.S. Provisional Application No. 63/678,843, filed Aug. 2, 2024, the entire disclosures of which are hereby incorporated herein by reference in their entirety.

### BACKGROUND

### 1. Technical Field

The field of the currently claimed embodiments of this invention relates to medical devices, and, more particularly, to delivery and anchoring medical devices within a hollow, substantially cylindrical organ cavity (such as, e.g., an intestine), a tissue cavity and/or a luminal tissue space using a specialized delivery system and endoscope.

### 2. Discussion of Related Art

Delivery and anchoring devices for medical devices exist. But, delivery and anchoring of medical devices within an organ cavity, tissue cavity and/or a luminal tissue space can be challenging. Improvement to existing medical device anchoring is needed, especially, in an organ cavity, a tissue cavity and/or luminal tissue space.

### SUMMARY

According to some embodiments of the invention, an anchoring system includes a sleeve having an inner surface defining an inner lumen, a first expandable sealing mechanism disposed along a proximal end of the sleeve, and a second expandable sealing mechanism disposed along the proximal end of the sleeve. The anchoring system further comprises a roughening material disposed on an outer surface of the sleeve and a sheath covering the outer surface of the sleeve, the roughening material, and the first and second expandable sealing mechanisms, wherein the sheath is configured to be removed after insertion of the device into a tissue cavity. Expansion of the first and second expandable sealing mechanisms and application of negative pressure to the anchoring system, causes a seal to form between the first and second expandable sealing mechanisms, the outer surface of the sleeve, and an inner surface of a tissue cavity.

In some embodiments, each of the first and second expandable sealing mechanisms comprises a low-profile balloon. In some embodiments, each of the first and second expandable sealing mechanisms comprises a balloon having a varying thickness, such that each of the first and second expandable sealing mechanisms has a higher thickness in an area of the respective balloon that attaches or bonds to the sleeve.

In some embodiments, the sheath comprises a smooth material.

According to one embodiment, the roughening material comprises an open-cell foam. According to some embodiments, the roughening material comprises at least one of a porous fiber or matrix, a polymeric material having small protrusions, an open-cell foam, or a combination thereof.

According to one embodiment, the anchoring system further includes a sleeve body. According to an embodiment, the first expandable sealing mechanism is disposed at a proximal end of the sleeve body and the second expandable sealing mechanism is disposed at a distal end of the sleeve body. According to some embodiments, the sleeve body and sleeve are comprised of the same extruded piece of polymer. According to other embodiments, the sleeve body is a separate tubular structure bonded to the tubular structure of the sleeve. According to some embodiments, the sleeve body of the anchoring system has thicker wall thickness than the sleeve of the system. According to some embodiments, the sleeve body of the anchoring system has a higher durometer than the sleeve of the system. According to some embodiments, the sleeve body of the anchoring system is configured so that the lumen does not collapse closed when the expandable sealing mechanisms are expanded within a tissue cavity and negative pressure is applied to the anchoring system. According to some embodiments, the sleeve body of the anchoring system is configured so that the expandable sealing mechanisms remain in a near perpendicular orientation to the sleeve body when the sealing elements are expanded within a tissue cavity and negative pressure is applied.

According to some embodiments, the distal end of the sleeve body is connected to a proximal end of the sleeve. According to another embodiment, the sleeve body is coextensive with the sleeve. According to some embodiments, the sleeve body is disposed on a proximal end of the sleeve.

According to an embodiment, the sleeve includes a plurality of fluid lumens. According to some embodiments, the plurality of fluid lumens includes one or more of (i) a distal fluid lumen to provide a fluid to the first expandable sealing mechanism, (ii) a proximal fluid lumen to provide a fluid to the second expandable sealing mechanism, (iii) a flushing lumen, (iv) a contrast dye lumen, and (v) a negative pressure lumen.

According to some embodiments of the invention, application of negative pressure creates a frictional force that resists displacement of the sleeve and/or the sleeve body. According to some embodiments of the invention, the application of negative pressure brings the roughening material disposed on the outer surface of the sleeve into contact with the inner surface of the tissue cavity thereby creating frictional force that resists displacement of the sleeve.

According to some embodiments of the invention, the first and second expandable sealing mechanisms are expanded by providing a non-compressible fluid (e.g., saline, mineral oil, and/or a dye-based (e.g., iodine-based) contrast solution) to inflate or expand the first and second expandable sealing mechanisms. According to some embodiments of the invention, the first and second expandable sealing mechanisms are expanded by a radiopaque liquid. According to some embodiments of the invention, the first and second expandable sealing mechanisms are expanded by filling with a non-compressible liquid. According to some embodiments the expandable sealing mechanisms are expanded by filling with air. According to an embodiment, once expanded, the first and second expandable sealing mechanisms form a substantially airtight and fluid-tight seal with the inner surface of the tissue cavity. According to some embodiments of the invention, the first and second expandable sealing elements (or mechanisms) are collapsible.

According to some embodiments of the invention, the first and second expandable sealing mechanisms are expanded to create a difference in luminal occupancy that results in a step off between the first and second expandable sealing elements and the inner lumen in an area in which the seal is formed between the first and second expandable sealing mechanisms, the outer surface of the sleeve, and the inner surface of the tissue cavity when negative pressure is applied, such that the inner surface of the tissue cavity (e.g., bowel lumen) conforms to this step off, and thereby displacement forces required to slide the device within the tissue cavity (e.g., bowel) are increased.

According to some embodiments of the invention, the inner lumen of the sleeve extends the length of the device and is configured to protect the inner surface of the tissue cavity from fecal flow distal to the sleeve body. According to some embodiments, the inner lumen has a diameter between approximately 1 cm and approximately 6 cm. According to an embodiment, the inner lumen is hollow. According to some embodiments, the sheath is disposed through the inner lumen and through an entire length of the device to a region of the device that is outside of the patient and at an opposite end of the inner lumen where the anchoring element (or sleeve) is disposed, such that the sheath everts back onto itself, and extends back down the length of the device on the outer surface, thereby covering the anchoring system. According to an embodiment, the sheath is configured to be removed by applying tension on the sheath at an end of the sheath that is opposite to the anchoring element (or sleeve) (i.e., the proximal end), thereby causing the sheath to de-evert, uncovering the anchoring element and allowing the sheath to be removed by pulling the sheath all the way through the inner lumen. According to some embodiments, the distal end of the sheath that is everted to cover the anchoring element (or sleeve) is connected mechanically to a filament or tube provided through the inner lumen that can be pulled on to provide traction and remove sheath to expose the anchoring element (or sleeve) of the device. In yet another embodiment, the sheath encases the outside surface of the device and is not withdrawn through the center of the anchoring element (or sleeve) but is directly withdrawn from the outside of the device to expose the anchoring element (or sleeve) of the device. According to an embodiment, a filament or tube is mechanically attached to a portion of sheath that covers the anchoring element (or sleeve) of the device and thereby provides a means of retracting the sheath and exposing the anchoring element (or sleeve) of the device.

According to an embodiment, a lubricant or other friction-reducing substance is provided along one or more of (i) inner and/or outer surfaces of the sheath (e.g., sliding surfaces), (i) an inner surface of the inner lumen, and (iii) the outer surface of the sleeve. In some embodiments, the lubricant or other friction-reducing substance comprises at least one of (i) a liquid, (ii) a surface coating, (iii) a solid particulate, or (iv) a combination thereof, which are pre-applied to the device during manufacturing and are already in place when a user is presented with the device for use. In some embodiments, the lubricant or other friction-reducing substance comprises a dry lubricant. According to an embodiment, the lubricant or other friction-reducing are provided with a packaging of the device and a user applies them to the device prior to use. In further embodiments, a first type of lubricant is provided between the outer surface of the sheath and the inner surface of the lumen and a second type of the lubricant, which is differ from the first type of lubricant, is provided between the inner surface of the sheath and the outer surface of the sleeve. In some embodiments, the same type of lubricant is used or only one of the surfaces is provided with a lubricant.

According to some embodiments, the outer surface of the sleeve and/or the sleeve body has a diameter between approximately 1.1 cm and approximately 6.1 cm. According to some embodiments, the sleeve and/or the sleeve body comprises a flexible material having a Shore A hardness between about 20A and about 70A. According to some embodiments, the sleeve and/or the sleeve body has a length that is between about 3 cm and about 25 cm. According to some embodiments, the sleeve and/or the sleeve body has a tubular wall thickness of between about 0.1 mm and about 8 mm. According to some embodiments, the sleeve and/or the sleeve body has a tubular wall thickness that is between about 0.2 mm and about 5 mm.

According to some embodiments of the invention, the roughening material comprises an open-cell foam that comprises a material having an average pore size between about 50 microns and about 1000 microns. According to some embodiments, the open-cell foam comprises a material having an average pore size between about 300 microns and about 600 microns. According to some embodiments, the open-cell foam comprises a material having an average pore size between about 200 microns and about 400 microns. According to some embodiments, the open-cell foam comprises a material having an average pore size between about 100 microns and about 300 microns. According to some embodiments, the open-cell foam is compressible by peristaltic contractions of a patient's bowel. According to some embodiments, the open-cell foam is compressible by negative pressure between the sealing elements, sleeve, and tissue cavity. According to some embodiments, the open-cell foam comprises polyvinyl alcohol, polyurethane foam, or other synthetic polymer. According to some embodiments, the open-cell foam has a tensile strength of at least 50 kpa. According to some embodiments, the open-cell foam has a thickness of between 2 mm and 150 mm. According to some embodiments, the open-cell foam comprises a single tubular piece of foam. According to some embodiments, the open-cell foam comprises multiple pieces of foam. According to some embodiments, the open-cell foam is bonded to the sleeve and/or the sleeve body. According to some embodiments, the open-cell foam has a higher coefficient of friction than that of the sleeve and/or the sleeve body.

According to some embodiments, the first and second expandable sealing mechanisms each comprises an inflatable or expandable elastomeric balloon. According to some embodiments, the first and second expandable sealing mechanisms each comprises multiple expandable elastomeric balloons. According to some embodiments, the first expandable sealing mechanism comprises a single inflatable or expandable elastomeric balloon, while the second expandable sealing mechanism comprises multiple or a plurality of expandable elastomeric balloons. According to an embodiment, the first and second expandable sealing mechanisms have an annular diameter that is greater than an annular diameter of the open-cell foam dispersed around the sleeve and/or the sleeve body.

According to some embodiments of the invention, the sleeve has a column strength of about 3.0 lbs. to 6.0 lbs. According to some embodiments of the invention, the sleeve has a column strength of about 3.0 lbs to 6.0 lbs. According to some embodiments the combined sleeve body and sleeve have a column strength of 3.0 lbs to 6.0 lbs.

According to some embodiments, the inner lumen of the sleeve remains patent throughout the length of the device to allow for constant decompression of the tissue cavity (e.g., bowel). According to an embodiment, the sleeve attaches to an effluence bag at a distal end of the sleeve, wherein the inner lumen of the sleeve remains patent along a length of the device and up to the effluence bag to allow for constant decompression of the tissue cavity (e.g., bowel). In some embodiments, the lumen is designed to remain patent to air and fluid despite compression from the anus.

According to some embodiments of the invention, the anchoring system further includes a shaft element that is disposed within the inner lumen of the sleeve and couples to the device, such that it may be used to advance the device to its desired position. In some embodiments, the shaft element is flexible enough to allow for delivery of the device through the torturous course of the intestines. According to an embodiment, once the device is in its desired position and the anchoring system is activated, the shaft can be removed, and all that is left in place is the soft, flexible anchoring system. In one embodiment, the sheath is provided on an outside surface of the shaft element. In some embodiments, the shaft is hollow and includes a lumen, such that the sheath is disposed within the lumen of the shaft (which itself is disposed within the inner lumen of the sleeve).

According to some embodiments, the shaft element includes at least one of an endoscope, a colonoscope, or a sigmoidoscope, upon which the shaft element is able to advance the device into a patient. This may aid in visualization of the treatment area or allow manipulation such as introduction of air to expand the cavity and make the insertion and advancement easier. In some instances, an endoscope may not be available, and thus a rigid shaft may be supplied with the device to be used. It would be further advantageous to have a single device configurable to use either the supplied semi-rigid shaft or an endoscope.

According to some embodiments of the invention, the sheath of the anchoring system is disposed within the inner lumen of the device, and is comprised of a thin, flexible, smooth, but not stretchable, material, disposed in a cylindrical form. According to an embodiment, on one side of the sheath, e.g., on the side opposite to the anchoring system side of the sheath, the sheath is coupled to a hollow cylindrical slider element, with this slider element disposed within a larger cylindrical handle. In some embodiments, the cylindrical handle is detachable and coupled to the end of the device. According to an embodiment, the cylindrical handle further comprises one or more cutouts disposed axially, wherein a member or portion of the slider element may protrude through the one or more cutouts. In some embodiments, a portion of the sheath disposed within the lumen of the device may comprise woven fibers, one or more discrete filaments, a flexible cylindrical tube structure, or a combination thereof.

In some embodiments, the sheath and slider element are joined mechanically together such that they create one continuous luminal path to the opposite end of the device. The semi-rigid shaft would then be placed through this luminal path, and in one embodiment, secured into place mechanically at the end of the handle element. In one embodiment, the securing is done by a snap fitting.

According to some embodiments of the invention, the anchoring system further includes a negative pressure source, wherein negative pressure is applied to the anchoring system by the negative pressure source to maintain one of a constant negative pressure or a variable negative pressure at a level between −50 mmHg and −200 mmHg and/or between −50 mmHg and −150 mmHg. According to an embodiment, the negative pressure source (e.g., pump) has electronic controls (e.g., a controller) that allow for the pump to maintain negative pressure in a continuous fashion in a range from around −50 mmHg to −150 mmHg. In some embodiments, the negative pressure source comprises an electrically powered negative pressure pump attached to a collection canister. In some embodiments, the pump has a sensor to indicate that negative pressure has dropped from a pre-determined setting range. According to one embodiment, the sensor includes a “low pressure” indicator or alarm. According to some embodiments, the pump includes one or more of (i) a full canister alarm, (ii) a low pressure alarm, (iii) a leak alarm, and (iv) an idle alarm. According to some embodiments, the pump includes a long-lasting battery.

According to an embodiment, the anchoring system includes a negative pressure lumen configured to provide negative pressure to the sleeve and/or the sleeve body. According to some embodiments of the invention, the anchoring system further includes an irrigation lumen in fluid connection with the outer surface of the sleeve and/or the sleeve body. According to some embodiments of the invention, the anchoring system further includes an irrigation system in fluid connection with the sleeve and/or the sleeve body, wherein the irrigation system introduces a fluid into the sleeve and/or the sleeve body for irrigation. In some embodiments, the irrigation tube in fluid connection with the outer surface of the sleeve is the same tubing as a pressure tube.

According to some embodiments of the invention, the sleeve has a length that allows it to extend outside the tissue cavity. According to some embodiments, the inner lumen and first and second expandable sealing mechanisms are compressible by normal peristaltic forces of a patient's bowel. According to some embodiments, a diameter of the first expandable sealing mechanism and the second expandable sealing mechanism is less than or equal to a diameter of the tissue cavity in which the sleeve is to be anchored. According to some embodiments, a diameter of the first expandable sealing mechanism and the second expandable sealing mechanism is greater than a diameter of the tissue cavity in which the sleeve is to be anchored, when the first expandable sealing mechanism and the second expandable sealing mechanism are in an expanded state. According to some embodiments, the anchoring system is configured so that traction on the sleeve can be used to remove the anchoring system from the body cavity. According to some embodiments, the sleeve has a wall thickness that is between about 50 microns and about 5 mm. According to some embodiments, the sleeve has a length that is between about 8 inches and about 72 inches. According to some embodiments, the sleeve has a length that is between about 3 cm and about 25 cm. According to some embodiments, the sleeve has markings along its length that indicate the length of sleeve within the tissue cavity after placement. According to some embodiments, the sleeve body and/or the sleeve is comprised of one or more of silicone, polyurethane, thermoplastic elastomer, rubber, or other polymer. According to an embodiment, the sleeve and the sleeve body are comprised of one continuous tubular extrusion.

According to some embodiments of the invention, a pressure tube is attached to the sleeve along its length. According to some embodiments, a pressure tube is disposed within a wall of the sleeve. According to some embodiments, a pressure tube is integrated into the sleeve and comprises a same material as the sleeve. According to some embodiments, a pressure tube is disposed within an additional lumen along the length of the sleeve.

According to some embodiments of the invention, the sleeve body and/or the sleeve are comprised of one or more of silicone, polyurethane, thermoplastic elastomer, rubber, rubber-like material, or other polymer.

According to some embodiments of the invention, the anchoring system further includes a plurality of pressure tubes in fluid connection with the outer surface of the sleeve and/or the sleeve body.

According to some embodiments of the invention, the anchoring system further includes an effluence bag in fluid connection with the sleeve, the effluence bag configured to receive the content of the sleeve. According to some embodiments, the effluence bag is detachable. In some embodiments, the effluence bag can be emptied of content without detachment from the sleeve.

According to some embodiments of the invention, the sleeve has a proximal end in sealed fluid communication with, and extending distal to, the distal end of the sleeve body and the second expandable sealing mechanism, wherein the proximal end of the sleeve, extending distal to the distal end of the sleeve body, is configured to cover and protect a damaged area of tissue of the tissue cavity from content flowing through the lumen of the sleeve and the sleeve body. According to some embodiments of the invention, the sleeve body is bonded around the sleeve. According to some embodiments the sleeve body and the sleeve of the anchoring system are a single continuous piece of polymer, such that the sleeve body and the sleeve share the same lumen.

According to some embodiments of the invention, the sleeve and/or the sleeve body and the first and second expandable sealing elements are made from a single injection mold using a single material.

According to some embodiments, the anchoring system is configured to be positioned in the tissue cavity using an endoscope.

According to some embodiments, the tissue cavity is bowel comprising an anastomosis, and wherein the anchoring system is positioned within the bowel such that the anastomosis is located distal in the bowel to the second expandable sealing mechanism. According to some embodiments, the anchoring system further includes an irrigation system in fluid connection with the sleeve and/or the sleeve body, wherein the irrigation system introduces a fluid into the sleeve and/or the sleeve body for irrigation.

According to some embodiments, there is an irrigation tubing that extends from outside the body to the bowel, proximal within the bowel to the first expandable sealing mechanism. In some embodiments, this irrigation tubing is used to dilute stool that impacts the proximal within the bowel end of the anchoring system.

According to some embodiments, there is an additional irrigation tubing that extends from outside the body to the bowel, distal within the bowel to the second expandable sealing mechanism. In some embodiments, this additional irrigation tubing is used to irrigate the anastomosis. In some embodiments, this additional irrigation tubing is used to inject contrast to check for an anastomotic leak with a radiographic study.

According to some embodiments of the invention, a delivery system includes an outer protective sheath that encases the anchoring system according to embodiments of the invention, a handle, a guide shaft, and a sheath pull handle. The anchoring system is configured to be pushed into position by advancing the guide shaft into a patient's bowel. According to some embodiments, the delivery system outer protective sheath is withdrawn through the center of the guide shaft to expose the first and second expandable sealing mechanisms and the roughening material (or open cell foam) disposed on the outer surface of the sleeve body. In some embodiments, the outer protective sheath only extends to cover the sleeve body of the anchoring system containing the first and second expandable sealing mechanisms and the roughening material.

According to some embodiments, a delivery system is provided that includes a handle, a guide shaft connected to the handle, an outer protective sheath, and a sheath pull handle attached to the outer protective sheath. According to an embodiment, the outer protective sheath is disposed within a central lumen of the guide shaft and is configured to extend from the central lumen in order to cover and protect a proximal end of an anchoring system having at least one expandable sealing mechanism. According to an embodiment, the delivery system is used with an anchoring system according to embodiments of the invention having a sleeve with a column strength that is high enough to prevent collapse of the system during insertion via the delivery system.

According to some embodiments of the invention, the roughening material comprises an air conducting rough surface material disposed on the outer surface of the sleeve body or proximal to the body portion of the sleeve. According to some embodiments, the air conducting rough surface material is a stacked mesh matrix, a honey-comb lattice of interconnected channels, gauze, fabric, or a three-dimensional woven material, which can be, e.g., oriented in a radial fashion around the sleeve.

According to some embodiments of the invention, a method for anchoring a sleeve in a tissue cavity is provided, with the sleeve having an outer surface an expandable sealing mechanism for isolating a portion of the tissue cavity adjacent to the sleeve from a remainder of the tissue cavity, includes inserting the sleeve in the tissue cavity. The method further includes inflating or expanding the expandable sealing mechanism to create a seal between the expandable mechanism and the inner surface of a tissue cavity, and applying a constant negative pressure in a range of −50 mmHg to −150 mmHg using an electrically powered negative pressure pump to a region between an outer surface of the sleeve and an inner surface of the isolated portion of the tissue cavity to create a frictional force between the outer surface of the sleeve and the inner surface of the tissue cavity. According to an embodiment, the sleeve further comprises a roughening material (e.g., open cell foam) for contacting an inner wall of the tissue cavity. According to one embodiment, the sleeve further comprises a sleeve body.

In some embodiments, the expandable sealing mechanism comprises a low-profile balloon. In some embodiments, the expandable sealing mechanism comprises a balloon having a varying thickness, such that the expandable sealing mechanism has a higher thickness in an area of the balloon that attaches or bonds to the sleeve.

According to some embodiments of the invention, the step of inflating or expanding the expandable sealing mechanism is conducted by injecting a non-compressible liquid into the expandable sealing mechanism. According to some embodiments of the invention, the step of inflating or expanding the expandable sealing mechanism is conducted by injecting an inflation media (e.g., saline, mineral oil, and/or a dye-based (e.g., iodine-based) contrast solution) into the expandable sealing mechanism. According to some embodiments, the step of inflating or expanding the expandable sealing mechanism is conducted by injecting radiopaque contrast into the expandable sealing mechanism.

According to some embodiments of the invention, the step of inserting the sleeve in the tissue cavity is conducted using a delivery system comprising an endoscope.

According to some embodiments of the invention, the step of inserting the sleeve in the tissue cavity is conducted using a delivery system that includes (i) a semi-rigid shaft element and/or (ii) a handle and a guide shaft. According to an embodiment, the method further includes a step of withdrawing the handle and the guide shaft of the delivery system from the sleeve.

According to some embodiments of the invention, the method further includes removing the sleeve from the tissue cavity by (i) releasing the negative pressure, (ii) collapsing the expandable sealing mechanism, and (iii) injecting an amount of saline through an irrigation tubing to break the seal.

According to some embodiments of the invention, an anchoring device is provided that utilizes two redundant methods of anchoring within the bowel, wherein the first method is to utilize a negative pressure based-friction anchor as described herein and the second method is to fixate the external portion of the device to skin with a column strength to the sleeve that is high enough to hold the anchor portion of the device in place even if there is failure of the first method of anchoring; and such that the first method of fixation is sufficient to hold the anchor portion of the device in place if there is failure of the second method. According to an embodiment of the invention, a method of inserting the anchoring system according to embodiments of the invention is provided that uses an endoscope that is connected to the anchoring system. According to some embodiments, the anchoring system is inserted using direct visualization via the endoscope.

According to some embodiments of the invention, an anchoring system includes a sleeve having an inner surface defining a lumen, a first expandable sealing mechanism disposed along a proximal end of the sleeve, and a second expandable sealing mechanism disposed along the proximal end of the sleeve. The anchoring system further comprises a roughening material disposed on the outer surface of the sleeve. Expansion of the first and second expandable sealing mechanisms and application of negative pressure to the anchoring system, causes a seal to form between the first and second expandable sealing mechanisms, the outer surface of the sleeve, and an inner surface of a tissue cavity. According to some embodiments, the system further includes a sleeve body, with the first expandable sealing mechanism disposed at a proximal end of the sleeve body and the second expandable sealing mechanism disposed at a distal end of the sleeve body.

According to some embodiments of the invention, an anchoring device configured to be anchored within a bowel of a patient is provided. The anchoring device comprises a sleeve configured to be positioned within the bowel of the patient, an external portion configured to extend externally from the bowel of the patient, and two redundant methods of anchoring the device within the bowel, wherein a first method of anchoring the device utilizes a negative pressure-based system that comprises an electrically powered negative pressure pump that applies negative pressure to the device to create a frictional force that resists displacement of the sleeve of the device from the bowel, wherein a second method of anchoring the device utilizes first and second expandable sealing mechanisms, wherein each of the first and second expandable sealing mechanisms comprises a low-profile balloon and expansion of the first and second expandable sealing mechanisms causes a seal to form between the first and second expandable sealing mechanisms and an inner surface of a tissue cavity, wherein at least one of (i) a column strength of the sleeve is high enough to hold the device in place even if there is failure of the first method of anchoring, (ii) the first method of anchoring is sufficient to hold the device in place even if there is failure of the second method of anchoring, or (iii) the second method of anchoring is sufficient to hold the device in place even if there is failure of the first method of anchoring.

According to an embodiment of the invention, an electrically powered negative pressure pump for use with an anchoring device configured to be anchored within a tissue cavity of a patient is provided. The anchoring device includes a sleeve having an outer surface for contacting an inner wall of the tissue cavity, and an expandable sealing mechanism for isolating a portion of the tissue cavity adjacent to the sleeve from a remainder of the tissue cavity. The electrically powered negative pressure pump comprises at least one pressure tube configured to connect to the sleeve of the anchoring device, to thereby provide negative pressure to the sleeve of the anchoring device to create a frictional force that resists displacement of the sleeve of the anchoring device from the tissue cavity, a collection canister configured to collect fluid from an area around the tissue cavity in which the anchoring device is anchored, and a negative pressure indicator configured to detect when the negative pressure provided to the sleeve of the anchoring device drops below a pre-determined threshold value, wherein the electrically powered negative pressure pump is configured to provide a continuous negative pressure to the sleeve of the anchoring device in a range of from −50 mmHg to −150 mmHg.

According to one embodiment, the at least one pressure tube is configured to connect to the sleeve of the anchoring device via an one-way valve.

According to one embodiment, the pump further comprises a controller configured to maintain the continuous negative pressure to the sleeve of the anchoring device.

According to one embodiment, the pump further comprises a housing configured to house electronic controls configured to maintain the continuous negative pressure to the sleeve of the anchoring device.

According to one embodiment, the sleeve further comprises a roughening material on the outer surface of the sleeve for contacting an inner wall of the tissue cavity.

According to some embodiments, the electrically powered negative pressure pump further includes one or more of (i) a full canister alarm, (ii) a leak alarm, and (iii) an idle alarm.

According to an embodiment, the pre-determined threshold value is −70 mmHg. According to an embodiment, the pre-determined threshold value is −60 mmHg. According to an embodiment, the pre-determined threshold value is −50 mmHg.

According to some embodiments, the negative pressure indicator includes an alarm comprising an audible signal. According to some embodiments, the negative pressure indicator comprises a display screen.

According to an embodiment, the collection canister comprises a housing that creates an internal chamber configured to hold the fluid collected from an area around or within the tissue cavity.

According to an embodiment, the collection canister further comprises an inlet into which the fluid collected from an area around the tissue cavity enters the internal chamber of the housing.

According to an embodiment, the electrically powered negative pressure pump includes a long-lasting battery.

According to one embodiment, the negative pressure brings the outer surface of the sleeve into contact with the inner wall of the tissue cavity thereby creating frictional force that resists displacement of the sleeve.

According to an embodiment of the invention, an anchoring system is provided that includes a sleeve having an inner surface defining an inner lumen, a first expandable sealing mechanism disposed along a proximal end of the sleeve, a second expandable sealing mechanism disposed along the proximal end of the sleeve, and a negative pressure system configured to provide negative pressure to the sleeve to cause the sleeve to remain in place in a tissue cavity, wherein expansion of the first and second expandable sealing mechanisms and application of negative pressure to the sleeve via the negative pressure system, creates a bypass for fluid, gas and/or solid matter flowing through the inner lumen of the sleeve such that a bolus of fluid, gas and/or solid matter has unrestricted flow down the sleeve to prevent build up of pressure or expansile forces in the bowel proximal to the sealing mechanism most proximal in the bowel (i.e., the first sealing mechanism). The sleeve is configured to allow unrestricted flow of fluid, gas, and/or solid material past the anal sphincter and into the collection bag, minimizing build up of pressure or enteric content volume that could result in enteric contents expanding the bowel wall and pushing past the sealing mechanism most proximal in the bowel (i.e., the first sealing mechanism). In some embodiments, the sleeve is configured to stent open the anal sphincter. In some embodiments, the sleeve is configured so that the pressure to expand the bowel wall larger than the diameter of the sealing mechanism most proximal in the bowel (i.e., the first sealing mechanism) is less than the pressure in the sleeve and collection bag so enteric contents preferably flow down the sleeve rather than expand the bowel wall. In some embodiments, the sleeve is configured to remain patent to air and fluid while implanted in in the tissue cavity. In some embodiments, the sleeve is configured to “decompress” the bowel proximal to the device to the atmospheric pressure outside the body. In some embodiments, the sleeve is configured to be open to air outside the body. In other embodiments, the sleeve is attached to an expandable collection bag. In some embodiments, the sleeve is configured to not collapse and become occluded by bending or the patient's weight on the sleeve. In some embodiments occlusion of the sleeve by bending or collapsing is prevented by raised feature or features on the inner surface of the sleeve.

According to an embodiment, the anchoring system further comprises a sheath covering an outer surface of the sleeve the first and second expandable sealing mechanisms. According to one embodiment, the sheath is configured to be removed via the inner lumen after insertion of the system into a tissue cavity.

According to an embodiment of the invention, an anchoring system is provided, with the anchoring system comprising a sleeve having an inner surface defining an inner lumen, a first expandable sealing mechanism disposed along a proximal end of the sleeve, a second expandable sealing mechanism disposed along the proximal end of the sleeve, a roughening material disposed on an outer surface of the sleeve, and a sheath configured to cover the outer surface of the sleeve, the roughening material, and the first and second expandable sealing mechanisms, wherein the sheath is configured to be removed via the inner lumen after insertion of the system into a tissue cavity, wherein the anchoring system comprises (i) a first configuration in which the sheath covers the outer surface of the sleeve, the roughening material, and the first and second expandable sealing mechanisms, and (ii) a second configuration in which the sheath is removed from the system after insertion of the system into a tissue cavity, and wherein expansion of the first and second expandable sealing mechanisms and application of negative pressure to the anchoring system, causes a seal to form between the first and second expandable sealing mechanisms, the outer surface of the sleeve, and an inner surface of a tissue cavity.

### BRIEF DESCRIPTION OF THE DRAWINGS

Further objectives and advantages will become apparent from a consideration of the description, drawings, and examples.

FIG. 1A is an illustration of an anchoring system according to some embodiments of the invention.

FIG. 1B is a schematic illustration of an anchoring system according to an embodiment of the invention.

FIG. 1C is a schematic illustration of an anchoring system according to an embodiment of the invention.

FIG. 2A is a schematic illustration of a sheath everted over or covering one end of an anchoring system according to some embodiments of the invention.

FIGS. 2B and 2C are schematic illustrations of applying tension to a sheath to uncover one end of an anchoring system according to some embodiments of the invention.

FIG. 3 is an illustration of an anchoring system and a delivery system that includes a handle with a rigid shaft for inserting the anchoring system according to some embodiments of the invention.

FIG. 4 is an illustration of a portion of an anchoring system, including a sheath coupled to a slider element, according to some embodiments of the invention.

FIG. 5 is a schematic illustration of a sheath everted over or covering one end of an anchoring system according to some embodiments of the invention.

FIGS. 6A and 6B are schematic illustrations of a semi-rigid shaft extending through an anchoring system according to some embodiments of the invention.

FIG. 7 is an illustration of an anchoring system being attached to a scope adapter mounted on an endoscope according to an embodiment of the invention.

FIG. 8 is a schematic illustration of an anchoring system having a negative pressure source attached to a negative pressure channel according to an embodiment of the invention.

FIGS. 9A and 9B are illustrations of a negative pressure system (e.g., pump and canister) for an anchoring system according to an embodiment of the invention.

FIG. 10 illustrates an exemplary tubing with connection members for connecting a negative pressure system (e.g., pump) to the anchoring system according to some embodiments of the invention.

FIG. 11A is a schematic illustration of a shaft tool for clearing obstructions according to an embodiment of the invention.

FIG. 11B is a schematic illustration of an anchoring system according to an embodiment of the invention.

FIG. 11C is a schematic illustration of a shaft tool in combination with an anchoring system according to an embodiment of the invention.

FIGS. 12A and 12B are schematic illustrations of a secondary lumen for connecting an external source to an anchoring system according to an embodiment of the invention.

FIG. 13A is a schematic illustration of a side view of an expandable sealing mechanism in the form of a low-profile balloon according to an embodiment of the invention.

FIG. 13B is a schematic illustration of a cross-sectional view taken along line A-A of FIG. 13A of the expandable sealing mechanism in the form of a low-profile balloon according to an embodiment of the invention.

FIG. 13C is a schematic illustration of an exploded view taken from area B of FIG. 13B of the expandable sealing mechanism in the form of a low-profile balloon according to an embodiment of the invention.

FIG. 13D is a schematic illustration of a top view of the expandable sealing mechanism of FIG. 13A in the form of a low-profile balloon according to an embodiment of the invention.

FIG. 14A is a graph illustrating the principal stress on an expandable sealing mechanism according to an embodiment of the invention.

FIG. 14B is a graph illustrating the principal strain on an expandable sealing mechanism according to an embodiment of the invention.

FIG. 15 is an illustration of an anchoring system in which negative pressure has been applied to create a seal between the anchoring system and the inner surface of a tissue cavity (e.g., bowel lumen) according to some embodiments of the invention.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure are intended to be illustrative, and not restrictive.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though they may. Furthermore, the phrases “in another embodiment” and “in some other embodiments” as used herein do not necessarily refer to a different embodiment, although they may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, terms such as “comprising” “including,” and “having” do not limit the scope of a specific claim to the materials or steps recited by the claim.

As used herein, terms such as “consisting of” and “composed of” limit the scope of a specific claim to the materials and steps recited by the claim.

All prior patents, publications, and test methods referenced herein are incorporated by reference in their entireties.

The need for temporary protection of the bowel lumen and/or a segment of the bowel from enteric fecal flow after surgical bowel resection and anastomosis or when the bowel wall is damaged (e.g., tissue damage) is a common problem in modern medical treatment. Examples include, e.g., protection of newly formed surgical anastomoses, anatomic leaks, inflamed or irritated bowel, partial thickness injuries to the bowel wall, and full thickness bowel perforations. A traditional means of temporarily protecting the bowel from enteric flow is by creating an external diversion of the bowel through the creation of an ostomy or stoma. An ostomy is a purposeful anastomosis between a segment of the gastrointestinal (GI) tract and the skin of the anterior abdominal wall. Basically, a surgeon diverts a segment of bowel proximal to the region to be protected to the skin, bypassing stool flow away from the vulnerable bowel segment. An ostomy can be created virtually anywhere along the GI tract. For diversion of the fecal stream, the most common ostomies involve the distal small intestine (e.g., ileostomy) and large intestine (e.g., colostomy). Ostomies are performed in around 300,000 patients in the US and over 2 million patients globally each year, but this surgery is complicated by high morbidity, mortality, and severe impact on a patient's quality of life. Complications such as, e.g., parastomal hernias, infections/sepsis, skin irritation and erosion, electrolyte depletion, dehydration, and prolapse are common. Up to 50% of patients who undergo ostomy surgery will have some type of ostomy related complication, with many requiring re-hospitalization or additional operations. Although many ostomies are intended to be temporary, as many as ⅓ of temporary ostomies are never reversed, due to, for example, the patient's fear of undergoing further surgery or risk factors associated with complications from the original surgery. In addition, the reversal surgery itself is associated with high complication rates (17%) and significant mortality (1.2-7%). Accordingly, there is a need for improved methods and devices to provide a less morbid alternative for fecal diversion for colonic protection.

One of the major indications for a temporary ostomy is to protect a bowel anastomosis after bowel resection surgery. The protective ostomy or stoma is performed to both protect the newly formed anastomosis from fecal flow and to prevent the development of severe sepsis if an anastomotic leak develops. An anastomotic leak is defined as a defect of the intestinal wall at the anastomotic site leading to a communication between the intra- and extraluminal compartments. The overall incidence of colorectal anastomotic leak varies widely in the literature, ranging from 1 to 24%. Leaks can cause severe complications such as loss of the anastomosis, sepsis, and death. Even in those cases where the anastomosis is salvaged, poor compliance in the neorectum can lead to a poor functional outcome. In many large studies, anastomotic leaks have been shown to be associated with a pelvic sepsis at a rate of 50%. By protecting the anastomosis from fecal flow, anastomotic leaks may be prevented, or perhaps more importantly, their complications of severe sepsis and death mitigated. This is why colon surgeons perform temporary ostomy surgeries after bowel resections even though the vast majority of patients will likely heal uneventfully. Even after an anastomotic leak has occurred, protection from fecal flow can make the anastomotic leak less severe and aid in healing of the leak. There are several risk factors for the development of an anastomotic leak. The most significant risk factor is the level of the anastomosis, with the leak rate increasing as the distance from the anastomosis to the anus decreases. Other than meticulous technique in creating the anastomosis, the major strategy to prevent and treat anastomotic leaks during complicated or high-risk cases involving bowel resection is to divert fecal flow with a protective ostomy. This is accomplished by having the flow of enteric contents diverted using an ostomy created in the bowel proximal to the anastomosis or anastomotic leak. Proximal in the bowel is defined as higher up in the GI tract towards the mouth, distal in the bowel is defined as lower down in the GI tract towards the anus. This ostomy can be either an end ostomy such as an end colostomy or end ileostomy or can be a diverting loop ileostomy that does not completely disrupt bowel continuity.

A temporary diverting ostomy and its closure has its own set of complications and morbidities including dehydration due to high output, difficulty with ostomy care, stricture at the closure site, wound infections, and incisional hernias. Complication rates of ostomies range between 5% and 100%. The complications can be divided into minor complications, which do not require surgical intervention, and major complications requiring surgical intervention. Major complications include stenosis, small bowel obstruction, retraction, necrosis, prolapse, stricture, fistula, and parastomal hernia. In some cases, such as partial small bowel obstruction, the patient can first be treated conservatively and surgical intervention may be avoided. Major complications such as ostomy necrosis that extends more than a few millimeters, surgical intervention is mandatory. Minor complications include dermatitis, electrolyte imbalance, and dehydration from high ostomy output, although the last often necessitates early closure of the ostomy. For major complications, additional costs and morbidity associated with additional operations or hospitalizations can be significant. Even for minor complications, treating complications and providing ostomy education can be burdensome to healthcare providers and patients. Some complications such as hernia, prolapse, and stenosis may become chronic and often require multiple corrective operations and associated costs. Ostomies also significantly reduce a patient's quality of life. Fecal output from the ostomy is collected into an ostomy bag attached to the patient's abdomen. These bags need to be emptied and replaced regularly to properly care for the ostomy and prevent unintentional discharge of fecal material. Ostomy appliances can fail and lead to leakage. The burden of living with an ostomy can negatively impact a patient's social life and restrict physical activities.

Furthermore, the reversal of an ostomy is a surgical procedure fraught with potential complications, as often times the abdominal compartment has dense adhesions that make re-establishment of normal bowel continuity both technically challenging and potentially morbid. The repaired bowel after ostomy takedown may also develop a leak at the repair site or anastomotic site in cases of loop ileostomies or in cases of end ostomy reattachment, respectively. Because of the potential for bowel injury and development of a leak from ostomy reversal surgery, the procedure is also associated with significant mortality. In addition to expenses associated with taking patients to the operating room, patients typically require a hospitalization of 2-4 days post-procedure until bowel function returns. Furthermore, the reversal of an ostomy may be too risky or impossible in some patients, requiring the patient to live the rest of their life with an ostomy.

Besides anastomotic protection, there are other potential indications for temporary fecal diversion. These include: 1) treatment of an anastomotic leak after it has occurred, 2) diverticulitis, 3) inflammatory bowel diseases such as Crohn's or Ulcerative Colitis, 4) intestinal perforation and 5) other less common instances of bowel injury where fecal diversion could be useful such as in cases of ischemic bowel disease, bowel contusion injury from trauma, or non-healing perineal/perianal wounds. When a leak or bowel perforation has occurred, such as, in cases of anastomotic leak and diverticulitis, as examples, treatment with fecal diversion can reduce the severity and extent of the condition. Thus, these patients may heal their leak/perforation faster and not develop more severe complications when continued fecal flow contamination of the affected site is mitigated. Inflammatory conditions of the bowel wall such as Crohn's disease or Ulcerative Colitis can make the intestinal lining susceptible to damage from fecal flow. Continued fecal flow can further inflame and contaminate the bowel wall and lead to worsening of the patient's overall disease, infection/sepsis, or even perforation of the bowel wall. Protection from fecal flow allows the inflamed sections of bowel to heal, and, potentially, fecal diversion could reduce recovery time, hospitalization time, and limit severe complications, such as, e.g., perforations or fistula formation. Patients with these conditions may not be good candidates for surgery due to their concomitant conditions or sepsis; thus, performing major surgery to create an ostomy can be morbid in these cases. Accordingly, there is a need for improved methods and devices to provide a less morbid alternative for temporary fecal diversion.

More recently, intraluminal diversion has been attempted using a protective sleeve/sheath within the bowel as an alternative to temporary ostomy formation. Instead of diverting stool outside the body with an ostomy/stoma, the stool is diverted through the lumen of the bowel inside a protective sleeve/sheath effectively protecting the bowel wall from fecal contamination. However, anchoring of the sleeve/sheath securely and safely within the bowel lumen has proven to be historically challenging. Previous attempts at anchoring within the bowel have all either caused potentially catastrophic complications (erosion or ischemic injury to bowel wall, increased anastomotic leak rates) or unreliable anchoring and protection (early extrusion, device migration, incomplete fecal bypass). Historically, these sleeves have been inserted during a laparoscopy or laparotomy under direct visualization or blindly without visualization. However, avoiding inadvertently causing damage to the bowel during blind insertion of a protective sleeve/sheath can be risky in the setting of bowel wall damage or other bowl pathology such as inflammation that can make the bowel wall more susceptible to iatrogenic injury. However, there are many clinical scenarios were placement of the anchor and attached sleeve is needed without an associated surgical laparoscopy or laparotomy. In these cases, performing a laparoscopy or laparotomy to assist in placement of the device would expose the patient to additional risks and potential complications from surgery that might limit the indications for use to only the most severe cases where the benefits of colonic protection outweighed the potential morbidity of abdominal surgery. The ability to anchor a protective sleeve/sheath safely without performing a laparoscopy or laparotomy would increase the indications for use of the protective device and minimize morbidity from using the device. Thus, there exists a need for a device to protect a segment of bowel that can be safely placed without laparoscopy or laparotomy through a completely intraluminal approach. There are many clinical examples where this technology could be beneficial. For example, conditions that require temporary colon protection could include, e.g., high risk colonoscopy, inflammatory bowel disease, or diverticulitis.

Disclosed herein are systems and methods for anchoring a protective sleeve within the bowel proximal to a region of bowel that requires protection from fecal flow, such as a bowel anastomosis or area of bowel damage. The system and methods can make temporary fecal diversion ostomy surgery unnecessary in most patients, as it provides internal fecal diversion and accomplishes the same overall objective as a temporary ostomy by protecting the distal segment of bowel from fecal flow. In addition, we disclose additional configurations of this system that enable drug delivery to the intestinal lumen.

According to an embodiment, an anchoring system or an Intraluminal Colonic Diversion (ICD) System is provided that is a medical device designed to temporarily protect a segment of colon from fecal flow. The system or product is intended to improve patient care by reducing the need for temporary ostomies after bowel wall damage. The ICD system is a two-part system that includes (i) a sterile, single patient use ICD Device and (ii) a sterile, single patient use ICD Delivery System. The ICD Device and ICD Delivery System are assembled ready-to-use in a pouch, such as, e.g., a Tyvek-PET/LDPE pouch, and the sealed pouch is packaged in a single chipboard box as the complete ICD System.

According to some embodiments, the system (i.e., the ICD System) includes an anchoring mechanism that allows for non-traumatic and reversible anchoring of a sleeve within the GI tract that diverts fecal contents away from the anastomotic site or area of damaged bowel. The device is designed to be left in place for a period of a few days to several weeks (including, e.g., 10 to 14 days, up to 21 days, and/or at least four weeks), and then removed completely from the patient after healing has occurred or diversion is no longer required. While the device and method are described here in the context of securely anchoring a sleeve within the GI tract for the purpose of therapeutic benefit such as diverting bowel contents, the device and method for anchoring may also have applications in other regions of the body where secure anchoring within a tissue cavity is desired. It is important to emphasize that this is a device designed to be substantially and securely anchored in place within the bowel and to prevent substantial device migration, including, during peristalsis of the bowel, until the device is actively disengaged and removed by the clinician. This is in contrast to other non-surgically attached sheath or sleeve-based protection devices that are unable to be securely anchored and are slowly extruded from the bowel over time because they cannot maintain the same high level of anchoring strength required to resist bowel expulsion forces (including, e.g., peristaltic forces). The unique design of the device disclosed herein allows for it to anchor in place within the bowel without dislodgement, without damaging the bowel wall, without the need for a surgical fixation such as suturing, stapling, or external to the bowel retention rings, and without the need for a permanent implant. Each of these features are described in more detail below.

According to an embodiment, the ICD device includes a specialized negative pressure anchor and an associated protective sleeve designed to reliably, reversibly, and safely protect the distal bowel (i.e., the bowel downstream in the gastrointestinal (GI) tract from the anchor) from fecal enteric flow. The specialized negative pressure anchor utilizes a unique foam interface (or a roughening material and/or an air conducting rough surface material) to create an atraumatic, but strong bond to the bowel wall that holds the ICD device in place and prevents migration. According to an embodiment, the anchor portion of the ICD device has expandable sealing mechanisms in the form of, e.g., two soft, inflatable elastomeric balloons that provide atraumatic sealing surfaces to both facilitate negative pressure anchoring and provide a fluid/air tight seal against fecal contamination distal within the bowel to the anchor portion. These seals are also collapsable to allow for withdrawal of the anchor system without straining the bowel wall. Negative pressure is applied to the sealed area between the expandable or expanded sealing mechanisms (e.g., the inflated balloons) and the bowel lumen to activate anchoring. A biocompatible elastomeric open-cell foam material and/or a roughening material is spaced between the expandable sealing mechanisms (e.g., balloons) to provide a large contact surface and a means for negative pressure to distribute evenly around the outer surface of the anchor sleeve and/or the anchor sleeve body and inner bowel wall. This interface also creates a unique friction bond to the bowel wall that is stronger than what has been previously achieved without more invasive fixation techniques. The ICD device is designed to be placed in the operating/procedure room and can be removed at the patient's bedside or in clinic. The ICD Device does not require surgical fixation, such as, e.g., stapling, extraluminal restriction rings, or expandable wire-stenting, that can damage the delicate bowel wall. The ICD Device can be left in place for typically a 10 to 14-day period, though it may be left up to 21 days or more if necessary, to provide extended protection to a surgical bowel anastomosis or segment of damaged bowel, such as, e.g., an anastomotic leak and/or inflamed bowel.

Further disclosed herein is a system or device intended to be placed within a hollow and substantially cylindrical organ cavity (such as, e.g., an intestine), with such device being capable of anchoring in place in a manner that is atraumatic and reliable. According to an embodiment, the device comprises an anchoring system that includes at least two expandable elements (such as, e.g., balloon seals) and a roughening material (such as, e.g., an open cell foam) disposed between the at least two expandable elements. When the anchoring system is to be activated or engaged with the organ cavity, the expandable elements are expanded, thereby coming into contact with the walls of the organ cavity and expanding the organ cavity. However, according to an embodiment, the section or portion of the anchoring system between the expandable elements where the roughening material is disposed is not in substantial contact with the walls of the organ cavity. A negative pressure is applied to the volume between the expandable elements, the wall(s) of the organ cavity (e.g., intestine), and the section of the anchoring system where the roughening material is disposed. This negative pressure pulls the walls of the hollow cylindrical organ cavity inward, into contact with roughening material. Thus, according to an embodiment, the anchoring system is held in place not substantially by any means of expansile properties of the anchoring system, but by the mechanical interlocking of the portions of wall(s) of the organ cavity that are in contact with the roughening material. In some embodiments, the roughening material is an open cell foam. In other embodiments, the roughening material is any porous fiber or matrix. In yet other embodiments, the roughening material is a polymeric material with small protrusions. According to some embodiments, the roughening material comprises at least one of a porous fiber or matrix, a polymeric material having small protrusions, an open-cell foam, or a combination thereof.

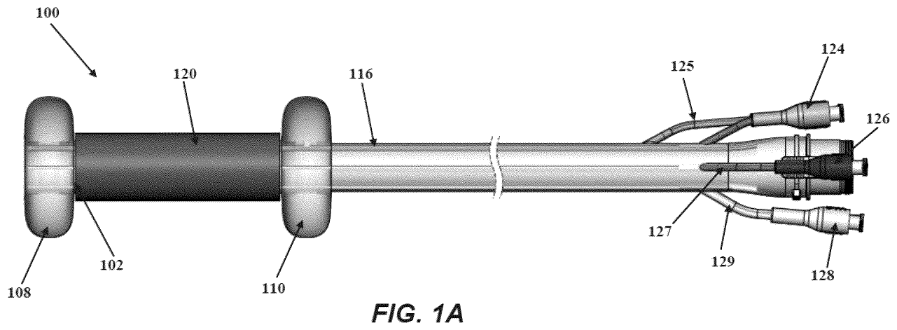

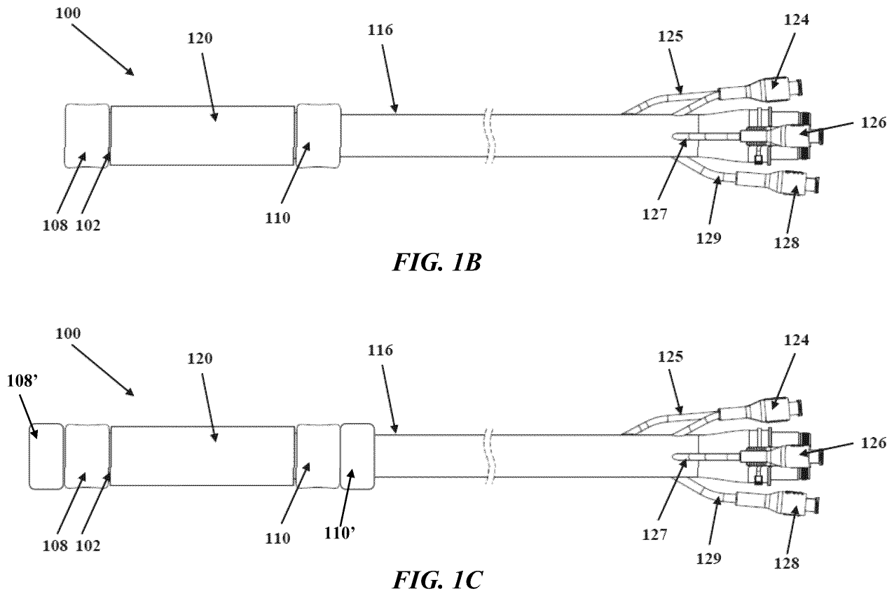

FIG. 1A shows an illustration of an anchoring system according to some embodiments of the invention. The anchoring system 100 includes a sleeve body 102 having an inner surface (not shown) defining a lumen (not shown). A first expandable sealing mechanism 108 is disposed at a proximal end of the sleeve body 102, and a second expandable sealing mechanism 110 is disposed at a distal end of the sleeve body 102. For the device, proximal is defined as the part of the device farthest from where fecal matter exits the sleeve body and/or the sleeve (at an effluence bag, for example), and distal is defined as the part of the device that is closer to where fecal matter exits the sleeve body and/or the sleeve during regular fecal flow. This orientation convention is used because this is the relationship of flow through the device (from proximal to distal) and matches the orientation of the device within the bowel. A sleeve 116 is in fluid communication with the sleeve body 102, and forms a second lumen (not shown) that is in continuity with the first lumen. A roughening material 120, such as, e.g., an open-cell foam, is disposed on the outer surface of the sleeve body 102. The application of negative pressure to the sleeve 116 and/or the sleeve body 102, as well as inflation of the first and second expandable sealing mechanisms 108, 110, causes a seal to form between the first and second expandable sealing mechanisms 108, 110 and an inner surface (or the inner walls) of the tissue or organ cavity, and creates a frictional force that resists displacement of the sleeve body 102. In some embodiments, the sleeve 116 extends through the sleeve body 102, such that the sleeve 116 is coextensive with the sleeve body 102. In another embodiment, the sleeve body 102 is absent and the expandable sealing mechanisms 108, 110 and the roughening material 120 are attached directly to a proximal end of the sleeve 116. In other embodiments, the distal end of the sleeve body 102 is connected to the proximal end of the sleeve 116.

Also shown in FIG. 1A are a series of flexible (or fluid) tubes 125, 127, and 129 and their associated connectors or ports 124, 126, 128, respectively. According to an embodiment, one of the flexible tubes 125 provides a fluid or inflation media (e.g., saline, mineral oil, and/or a dye-based (e.g., iodine-based) contrast solution), e.g., through its associated connector or port 124, to each of the first and second expandable sealing mechanisms 108, 110, in order to inflate the first and second expandable sealing mechanisms 108, 110 with the fluid or inflation media and create a seal between the first and second expandable sealing mechanisms 108, 110 and an inner surface (or the inner walls) of the tissue or organ cavity. Alternatively, the system 100 may include two flexible tubes (not shown), one for each of the first expandable sealing mechanism 108 and the second expandable sealing mechanism 110 to provide a fluid or inflation media (e.g., saline, mineral oil, and/or contrast dye) for inflation. According to one embodiment, a flexible tube or tubing 127, which is in communication with a connector or port 126, can be used to introduce fluid from a fluid infiltration source such, e.g., a syringe. This port 126 can be accessed from outside the patient's body via the flexible tubing 127. This configuration of the anchoring system 100 allows for delivery and removal of irrigation, drugs (such as antibiotics, anti-inflammatory drugs, or chemotherapy agents), and radiologic contrast between the external surface of the sleeve 116 and bowel wall and between the two expandable sealing mechanisms 108, 110. For example, according to one embodiment, the flexible tube 127 and port 126 are provided that can be used to inject contrast dye or radiopaque dye distal to the anchor portion of the device within the bowel to perform a radiographic leak test. According to an embodiment, another flexible tube or tubing 129, such as, e.g., an irrigation tubing, is provided that is used (if necessary) to instill fluid, such as, e.g., normal saline, proximal to the anchor portion of the device to liquify formed stool and maintain patency of the sleeve body 102 and/or the sleeve 116. For example, according to one embodiment, a fluid, such as, e.g., saline, can be introduced via the flexible tube or tubing 129, which is in communication with a connector or port 128, from a fluid infiltration source such, e.g., a syringe, between the external surface of the sleeve 116 and bowel wall between the two expandable sealing mechanisms 108, 110. Alternatively, the flexible tube or tubing 129 can be used to introduce a negative pressure source (i.e., vacuum) to the sleeve 116 and/or the sleeve body 102 in order to cause a seal to form between the first and second expandable sealing mechanisms 108, 110 and an inner surface (or the inner walls) of the tissue or organ cavity.

FIG. 1B shows another illustration of the anchoring system 100 of FIG. 1A according to an embodiment of the invention. As shown in FIG. 1B, the anchoring system 100 includes the sleeve body 102. The first expandable sealing mechanism 108 is disposed at a proximal end of the sleeve body 102, and the second expandable sealing mechanism 110 is disposed at a distal end of the sleeve body 102. The sleeve 116 is in fluid communication with the sleeve body 102 by being connected to, coextensive with, and/or disposed within the sleeve body 102, with each of the sleeve 116 and the sleeve body 102 being in fluid connection with a lumen. The roughening material 120 (e.g., open-cell foam) is disposed on the outer surface of the sleeve body 102. As discussed above, the application of negative pressure to the anchoring system 100, as well as inflation of the first and second expandable sealing mechanisms 108, 110, causes a seal to form between the first and second expandable sealing mechanisms 108, 110 and an inner surface (or inner walls) of the tissue or organ cavity, and creates a frictional force that resists displacement of the sleeve body 102. Also shown in FIG. 1B, are the series of flexible (or fluid) tubes 125, 127, and 129 and their associated connectors or ports 124, 126, 128, respectively, that are provided to introduce a fluid or inflation media, such as, e.g., saline, mineral oil, contrast dye, etc., to the sleeve 116 and/or the sleeve body 102 via the flexible tube or tubing 125, 127, 129, which is in communication with a respective connector or port 124, 126, 128.

As shown in the embodiment of FIG. 1A, the first expandable sealing mechanism 108 disposed at the proximal end of the sleeve body 102, and the second expandable sealing mechanism 110 disposed at the distal end of the sleeve body 102 are in an expanded state (i.e., the first expandable sealing mechanism 108 and the second expandable sealing mechanism 110 have a diameter that is greater than a diameter of the sleeve body 102 and/or the sleeve 116). By contrast, as shown in the embodiment of FIG. 1B, the first expandable sealing mechanism 108 disposed at the proximal end of the sleeve body 102, and the second expandable sealing mechanism 110 disposed at the distal end of the sleeve body 102 are in an unexpanded or deflated state (i.e., the first expandable sealing mechanism 108 and the second expandable sealing mechanism 110 have a diameter that is equal to a diameter of the sleeve body 102 and/or the sleeve 116).

As further shown in the embodiment of FIGS. 1A and 1B, the first expandable sealing mechanism 108 disposed at the proximal end of the sleeve body 102, and the second expandable sealing mechanism 110 disposed at the distal end of the sleeve body 102 are centralized on the inner lumen (not shown) of the sleeve body 102, which thereby prevents occlusion of the inner lumen of the sleeve body 102.

FIG. 1C shows another illustration of the anchoring system 100 of FIGS. 1A and 1B according to an embodiment of the invention. As shown in FIG. 1C, the anchoring system 100 includes the sleeve body 102. The first expandable sealing mechanism 108 is disposed at a proximal end of the sleeve body 102, and the second expandable sealing mechanism 110 is disposed at a distal end of the sleeve body 102. According to this embodiment, additional expandable sealing mechanisms are included, such that a third expandable sealing mechanism 108′ is disposed at the proximal end of the sleeve body 102, and a fourth expandable sealing mechanism 110′ is disposed at the distal end of the sleeve body 102. The sleeve 116 is in fluid communication with the sleeve body 102 by being connected to, coextensive with, and/or disposed within the sleeve body 102, with each of the sleeve 116 and the sleeve body 102 being in fluid connection with a lumen. The roughening material 120 (e.g., open-cell foam) is disposed on the outer surface of the sleeve body 102. As discussed above, the application of negative pressure to the anchoring system 100, as well as inflation of the first, second, third and fourth expandable sealing mechanisms 108, 108′, 110, 110′ causes a seal to form between the first, second, third and fourth expandable sealing mechanisms 108, 108′, 110, 110′ and an inner surface (or inner walls) of the tissue or organ cavity, and creates a frictional force that resists displacement of the sleeve body 102. Also shown in FIG. 1C, are the series of flexible (or fluid) tubes 125, 127, and 129 and their associated connectors or ports 124, 126, 128, respectively, that are provided to introduce a fluid or inflation media, such as, e.g., saline, mineral oil, contrast dye, etc., to the sleeve 116 and/or the sleeve body 102 via the flexible tube or tubing 125, 127, 129, which is in communication with a respective connector or port 124, 126, 128.

According to some embodiments, the device includes a negative pressure based anchor that prevents a sleeve and/or a sleeve body from becoming dislodged from the inner surface of the bowel. According to some embodiments, the sleeve includes a sleeve body and/or is connected to a sleeve body and acts in combination as a protective barrier between the GI tract and the GI contents flowing through the sleeve and sleeve body. In some embodiments, the sleeve body is added onto a proximal end of the sleeve. In some embodiments, the sleeve and sleeve body are manufactured as one continuous tubular structure. In some embodiments, the sleeve and sleeve body are separate tubular components that are bonded together. According to one embodiment, the sleeve body is a reinforcing member designed to support the proximal end of the sleeve and/or device and, more specifically, the anchor element. The sleeve body is a key component to functionality of the device and (1) provides added support to allow for expansion of the expandable sealing members (e.g., balloons) without collapse of the sleeve inner lumen, (2) helps maintain the expandable sealing members (e.g., balloons) at an angle perpendicular to the sleeve and provides support to prevent them from twisting sideways as they expand, and (3) maintains an open and patent central lumen of the sleeve at the proximal end by providing necessary added support/strength to the sleeve. This allows for a thinner, softer and more flexible sleeve conduit along the length of the device.

According to some embodiments, the inner or central lumen of the sleeve and/or sleeve body remains patent throughout the length of the device to allow for constant decompression of the tissue cavity (e.g., bowel). According to an embodiment, the sleeve attaches to an effluence bag at a distal end of the sleeve, wherein the inner or central lumen of the sleeve and/or sleeve body remains patent along a length of the device and up to the effluence bag to allow for constant decompression of the tissue cavity (e.g., bowel). By providing this constant decompression of the tissue cavity, a bolus or bulkhead of fluid (e.g., GI contents) traveling through the tissue cavity (e.g., bowel) will also be decompressed and thus will not build up to a point that overcomes the seal created by the anchoring device with the inner wall of the tissue cavity. For example, if the inner or central lumen of the sleeve and/or sleeve body is pinched, restricted and/or closed at the distal end of the anchoring device (e.g., near the anal sphincter), any bolus or bulkhead of fluid traveling through the lumen of the device will be pushed back up the device (i.e., toward the proximal end of the device), which can cause failure of the device by, e.g., causing the seal created by the anchoring device with the inner wall of the tissue cavity to fail. Thus, by creating the inner or central lumen of the sleeve and/or sleeve body to be patent along a length of the device, the inner or central lumen of the sleeve and/or sleeve body remains open along the length of the device and/or the bowel to the outside of the body (e.g., the effluence bag), which provides a path for any bolus or bulkhead of fluid traveling through the lumen of the device to exit the device and further allows for this constant decompression of the tissue cavity (e.g., bowel). Such decompression of the tissue cavity (e.g., bowel) also provides relief to the patient by preventing the feeling of discomfort and/or bloating.

In accordance with the features of the embodiments of the invention, the device for anchoring the sleeve and/or the sleeve body within the bowel can be described as having a hollow body that includes an inner or central lumen with one or more expandable sealing elements on one or both ends and a roughening or porous material on the external surface of the hollow body, such that upon application of negative pressure to the external surface of the hollow body and expansion of the one or more expandable sealing elements, an adhesive force forms between the bowel wall and hollow body. According to one embodiment, the one or more expandable sealing elements are provided on both ends of the hollow body in order to centralize the inner lumen and prevent occlusion of the inner lumen. At least one lumen can deliver negative pressure (e.g., vacuum) to the sealing member, while one or more other lumens can deliver a fluid (e.g., saline, mineral oil, and/or a dye-based (e.g., iodine-based) contrast solution) to inflate the one or more expandable sealing elements. A protective sleeve can be attached to the sealing member and a collection system can collect contents which pass through the sealing member.

According to some embodiments, the anchoring portion of the device is positioned in the GI tract on the proximal side of an anastomosis or proximal to the area of damaged bowel. The anchoring portion of the device is defined as the portion containing the expandable sealing elements and foam (or roughening material) interface. The proximal side is the side that is “upstream” in terms of the flow of GI content through the GI tract. This is in contrast to anastomosis or wound treatment systems that are configured to be applied directly to an anastomosis or wound site. This device is configured to be anchored in healthy undamaged bowel. Constant and/or variable negative pressure is maintained via a pneumatic interface connected to the anchoring system and dispersed through an open cell reticulated foam interface (or a roughening material or an air conducting rough surface materials). Expandable sealing elements at the end of the sleeve and/or the sleeve body create a negative pressure space between the outer surface of the sleeve and/or the sleeve body containing the foam interface (or roughening material) and the bowel wall. When negative pressure is applied, the pressure gradient acts through the foam (or roughening material) to create adhesive and friction forces between the GI tract and the anchoring system. These adhesive and friction forces created by the negative pressure-sponge interface enable the anchoring system to maintain a relatively fixed position in the bowel that is much greater than other non-surgically fixated sleeve anchoring systems previously described. This fixation is also substantially safer with respect to bowel damage compared to other forms of fixation such as stapling, suturing, expandable stenting, or external fixation rings. When a user is ready to remove the device, normal atmospheric pressure between the anchoring device and bowel can be reestablished, allowing the device to move through the GI tract with minimal friction. The device and method of fixation do not require suturing, stapling, biodegradable implants, external fixation rings, or other invasive anchoring techniques, and create minimal trauma to the bowel. Thus, disclosed herein are a method and device for securely fixing a sleeve and/or a sleeve body within the bowel lumen in a manner that does not substantially damage the bowel wall, and that allows fixation to be easily reversed for device removal.

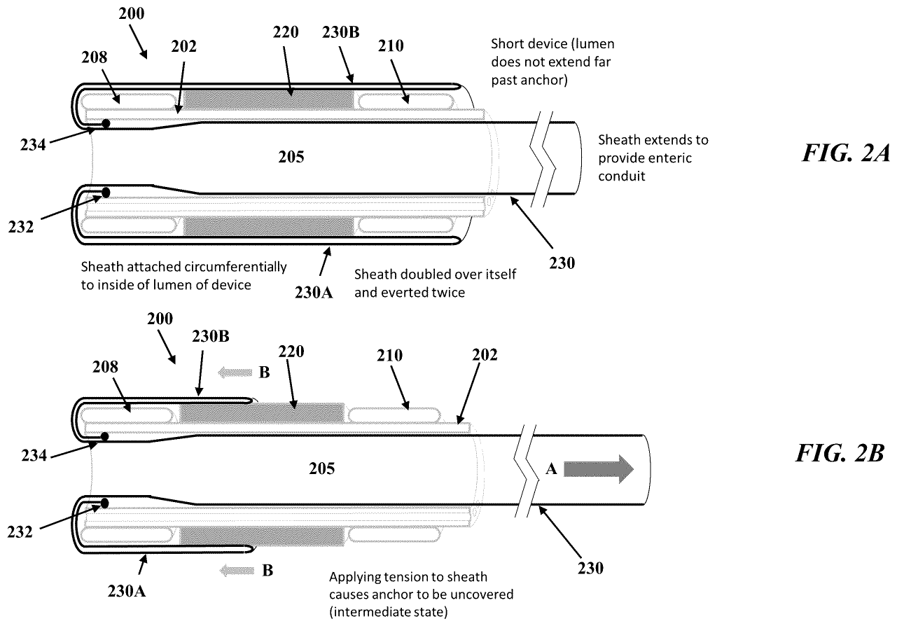

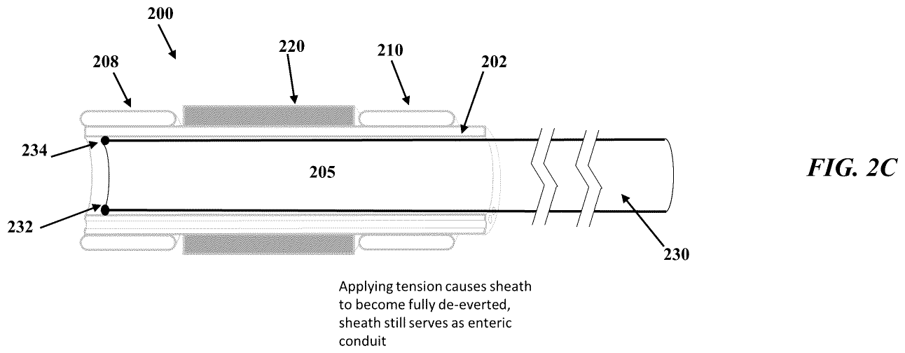

In advancing the anchoring system through the tissue or organ cavity to its site of deployment, it may be necessary to pass through an anastomosis, or other region of compromised tissue that the device may be intended to protect. In the case of an anastomosis, there may be metallic staples disposed circumferentially through the tissue or organ cavity that must be passed through and contact with a sharp end of a staple may damage the anchoring device. Furthermore, prior to expansion of the expandable elements (e.g., balloons), the highest profile area may be the roughening material (e.g., open cell foam), which may increase friction and thus cause difficulty advancing the anchoring device within the hollow organ cavity. Therefore, it is desirable to provide a smooth sheath that covers the anchoring system as it is being advanced to its desired position, and then, prior to activation of the anchoring system or expansion of the expandable elements (e.g., balloons), to remove the sheath. For example, by providing a sheath the covers the anchoring system when it is being advanced, the friction created by the roughening material (e.g., open cell foam) and the inner surface of the tissue or organ cavity is prevented and/or minimized, and the sheath further protects the anchoring system from any staples disposed circumferentially through the damaged tissue or organ cavity. In addition, the anchoring device comprises a hollow lumen through which intestinal contents are meant to pass through, when the anchoring device is placed or disposed internally in the tissue or organ cavity. According to one embodiment, the sheath may be disposed through the entire length of the device (i.e., through the hollow or internal lumen) to the region or portion of the device that is outside of the patient, and at the opposite end of the internal lumen where the anchoring system is disposed, to evert back on itself, and extend back down the length of the device on the exterior, thereby covering the anchoring system. For example, FIGS. 2A-2C show an illustration of an anchoring system 200 having a sheath 230 according to an embodiment of the invention. As shown in FIG. 2A, the anchoring system 200 includes a sleeve body 202 having an inner lumen 205. A first expandable sealing mechanism 208 is disposed at a proximal end of the sleeve body 202, and a second expandable sealing mechanism 210 is disposed at a distal end of the sleeve body 202. A roughening material 220 (e.g., open-cell foam) is disposed on the outer surface of the sleeve body 202. Positioned within the inner lumen 205 of the sleeve body 202 is a sheath 230, which is attached circumferentially at attachments points 232, 234 to inside of the inner lumen 205 of the sleeve body 202. The sheath 230 extends outside of the inner lumen 205 and the sleeve body 202 in order to cover at least a portion of the outside surface of the sleeve body 202, the first expandable sealing mechanism 208, the second expandable sealing mechanism 210, and the roughening material 220 at the proximal end of the sleeve body 202. As shown in FIG. 2A, which is the position of the sheath 230 during placement of the sleeve body 202 within a tissue or organ cavity, the sheath 230 doubles over itself at portions 230A, 230B in order to cover the outer portion of the sleeve body 202 having the first expandable sealing mechanism 208, the second expandable sealing mechanism 210, and the roughening material 220, and everts back over itself in order to extend back into the inner lumen 205 of the sleeve body 202. The sheath 230 extends through the inner lumen 205 to provide an enteric conduit for enteric (or GI) matter or contents flowing through the sleeve body 202 when in use.

In this embodiment, applying tension on the sheath 230 at its opposite end would cause it to de-evert, uncovering the anchoring element 200 and allowing the sheath 230 to be removed by pulling it all the way through the lumen 205. For example, as shown in FIGS. 2B and 2C, tension is applied to the sheath 230 in the direction of arrow A, which causes the sheath 230 to be pulled through the inner lumen 205 and to expose or uncover the outer portion of the sleeve body 202 having the first expandable sealing mechanism 208, the second expandable sealing mechanism 210, and the roughening material 220. FIG. 2B shows an intermediate state or position of the sheath 230 in which tension is applied to the sheath 230 in the direction of arrow A, which causes the portion of the sheath 230 (i.e., portion 230A, 230B) that is covering the outer portion of the sleeve body 202 having the first expandable sealing mechanism 208, the second expandable sealing mechanism 210, and the roughening material 220 to move in the direction of arrows B and to begin to expose or uncover the outer portion of the sleeve body 202 having the first expandable sealing mechanism 208, the second expandable sealing mechanism 210, and the roughening material 220. FIG. 2C shows a final state or position of the sheath 230 in which tension has been applied to the sheath 230 in the direction of arrow A, which causes the sheath 230 to be fully de-everted and positioned within the inner lumen 205, such that the outer portion of the sleeve body 202 having the first expandable sealing mechanism 208, the second expandable sealing mechanism 210, and the roughening material 220 is completely exposed or uncovered. The position of the sheath 230 in FIG. 2C occurs when the anchoring element is positioned within the tissue or organ cavity and is now able to be activated and/or the first expandable sealing mechanism 208 and the second expandable sealing mechanism 210 can be expanded to engage with the inner walls of the tissue or organ cavity. In the intermediate and final positions (FIGS. 2B and 2C), the sheath 230 extending through the inner lumen 205 still provides an enteric conduit for enteric (or GI) matter or contents flowing through the sleeve body 202 when in use. By pulling the sheath 230 through the inner lumen 205, the surface tension that can occur between the sheath 230 and the inner surface of the tissue cavity when removing the sheath 230 is minimized and/or avoided. This allows for the removal of the sheath 230 from the outer portion of the sleeve body 202 having the first expandable sealing mechanism 208, the second expandable sealing mechanism 210, and the roughening material 220 to be easier.

In other embodiments, the distal end of the sheath that is everted to cover the anchoring system is connected mechanically to a filament or tube that can be pulled on to provide traction and removal of the sheath to expose the anchoring portion of the device. In yet other embodiments, the smooth sheath encases the outside of the device and is not withdrawn through the center of the anchoring but is directly withdrawn from the outside of the device to expose the anchor portion of the device. This embodiment may also have a filament or tube that is configured to be mechanically attached to the portion of sheath that covers the anchor portion of the device and can provide a means of retracting the smooth sheath.

According to some embodiments, it may further be advantageous to provide a lubricant or other friction-reducing substance between the sliding surfaces related to the sheath, the inner surface of the primary lumen (see, e.g., inner lumen 205 of FIGS. 2A-2C) of the anchoring device, and the outer surface of the sleeve or sleeve body in order to improve placement of the device within a patient. In some embodiments, the lubricants may be either liquids (such as, e.g., K-Y JELLY®), surface coatings, solid particulates or a combination thereof, which are pre-applied to the packaged device in manufacturing and are already in place when the user is presented with the anchoring device for use. In some embodiments, the lubricant is a dry lubricant that is disposed along at least the sheath. In other embodiments, these lubricants are provided with the packaging and the user applies them to the device prior to use. In further embodiments, the lubricant between the outer surface of the sheath and the inner surface of the lumen is different than the lubricant between the inner surface of the sheath and the outer surface of the sleeve or sleeve body. In some embodiments, the same lubricant is used, or only one of the surfaces is provided with a lubricant. In some embodiments, the lubricant that is applied between (i) the outer surface of the sheath and the inner surface of the lumen and/or (ii) the inner surface of the sheath and the outer surface of the sleeve or sleeve body, should not be applied in an excessive amount in order to prevent the lubricant from blocking the primary lumen (see, e.g., inner lumen 205 of FIGS. 2A-2C).

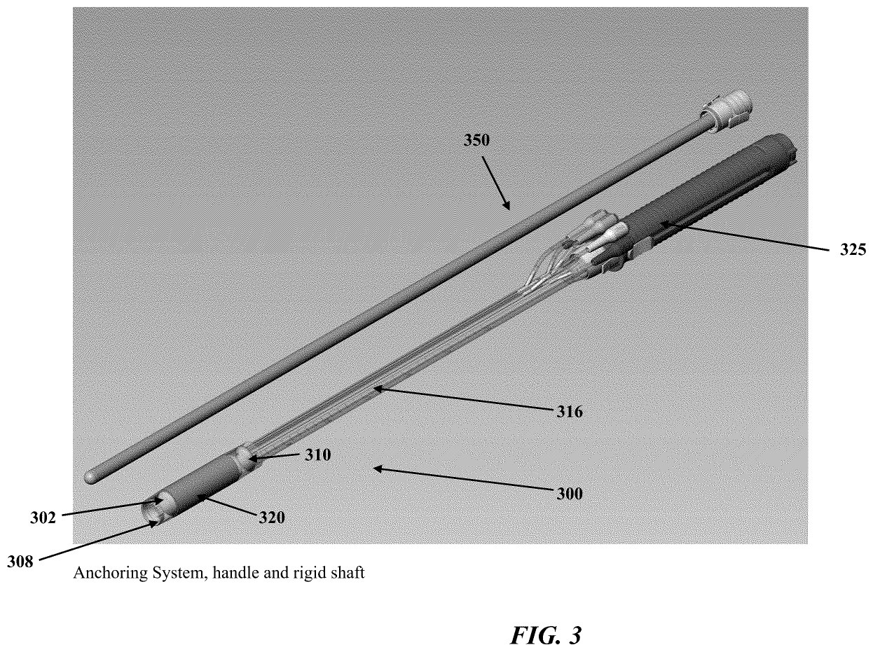

According to one embodiment, the implanted anchoring device is relatively soft and flexible, but includes a stiffness that is necessary to advance the device to its desired position. In some embodiments, there is a semi-stiff shaft element that is disposed within the lumen of the device that couples to the device, such that it may be used to advance the device to its desired position. This shaft in some embodiments is also configured to be flexible enough to allow for delivery of the device through the torturous course of the intestines. Once the device is in its desired position and the anchoring system is activated, the shaft can be removed, and all that is left in place is the soft, flexible device. For example, FIG. 3 illustrates an anchoring system 300 next to a rigid shaft 350, which can be positioned within the lumen of the anchoring system 300, such that the rigid shaft 350 may be used to advance the anchoring system 300 to its desired position within a tissue or organ cavity. As further shown in FIG. 3, the anchoring system 300 includes a sleeve body 302 having an inner surface (not shown) defining a lumen (not shown). A first expandable sealing mechanism 308 is disposed at a proximal end of the sleeve body 302, and a second expandable sealing mechanism 310 is disposed at a distal end of the sleeve body 302. A sleeve 316 is in fluid communication with the sleeve body 302, and forms a second lumen (not shown) that is in continuity with the first lumen. A roughening material 320, such as, e.g., an open-cell foam, is disposed on the outer surface of the sleeve body 302. The anchoring system 300 further includes a handle 325 that allows for a user to hold the anchoring system while positioning the anchoring system within the tissue or organ cavity. According to one embodiment, the rigid shaft 350 is configured to attach to an endoscope. According to another embodiment, the rigid shaft 350 comprises an endoscope.

In some embodiments, the smooth sheath described above is on the outside of the substantially stiff shaft. In some embodiments, the shaft is hollow and the smooth sheath is disposed within the hollow lumen of the shaft (which itself is disposed within the hollow lumen of the anchoring system). It may be advantageous to use an endoscope, such as a colonoscope or sigmoidoscope, as the semi-rigid shaft on which to advance the device. This may aid in visualization of the treatment area, or allow manipulation such as introduction of air to expand the cavity and make the insertion and advancement easier. In some instances, an endoscope may not be available, and thus a rigid shaft may be supplied with the device to be used (see, e.g., FIG. 3). It would be further advantageous to have a single device configurable to use either the supplied semi-rigid shaft or an endoscope.

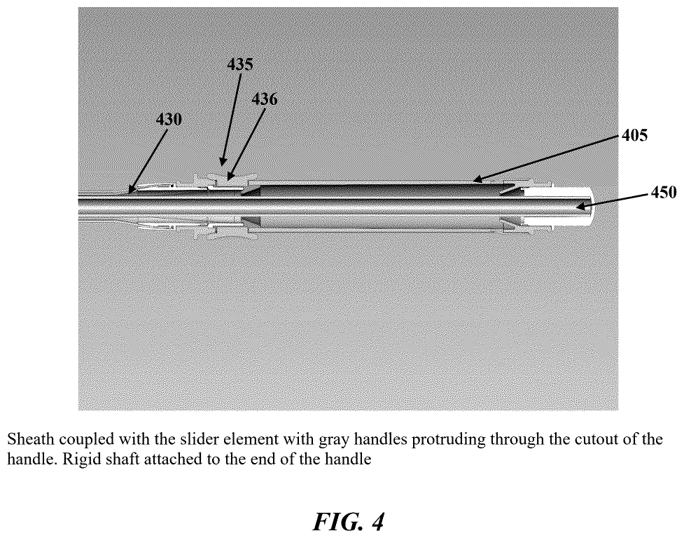

In one embodiment, the covering sheath disposed within the inner lumen of the anchoring device (see, e.g., sheath 230 in FIGS. 2A-2C) is comprised of a thin, flexible, smooth but preferentially not stretchable material, disposed in a cylindrical form. On the anchoring system side of the sheath, it is disposed as described above (see, e.g., FIGS. 2A-2C); on the other side of the sheath, it is coupled to a hollow cylindrical slider element, with this hollow cylindrical slider element itself disposed within a larger cylindrical handle. For example, FIG. 4 illustrates an embodiment of a sheath 430 coupled to a cylindrical slider element 435 having a handle 436, which is disposed within a cylindrical handle 405. As further shown in FIG. 4, a rigid shaft 450 is attached to the end of the handle 405. In some embodiments, the cylindrical handle 405 is detachable and coupled to the end of the anchoring device and/or an endoscope. This allows for the implantable device, sheath, and handle to be advanced and withdrawn as a single unit.

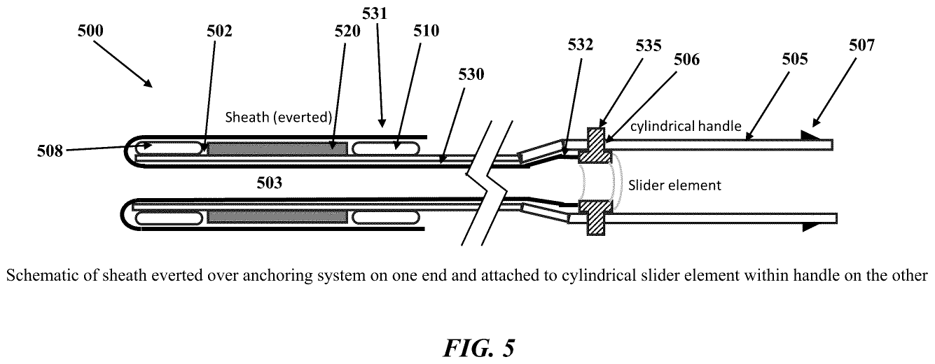

The cylindrical handle further comprises one or more cutouts disposed axially, wherein a member of part of the slider element may protrude through the cutouts (see, e.g., FIG. 5 described below). These members may then be manipulated to slide the slider element axially within the handle, transferring tension along the sheath and causing the sheath to uncover the anchoring system. The length of travel of the sheath would be set by the length of the cutout in the handle, and would be sufficient to completely uncover the anchoring system. For example, FIG. 5 illustrates an embodiment of a sheath 530, which has one end 531 covering and/or everted over an anchoring system 500 and a second end 532, which is attached to a cylindrical slider element 535 that is disposed within a cylindrical handle 505 but extends through a cut-out 506 provided in the cylindrical handle 505. In the embodiment of FIG. 5, the anchoring system 500 includes a sleeve body 502 having an inner surface defining an inner lumen 503, a first expandable sealing mechanism 508 that is disposed at a proximal end of the sleeve body 502, a second expandable sealing mechanism 510 that is disposed at a distal end of the sleeve body 502, and a roughening material 520, such as, e.g., an open-cell foam, that is disposed on the outer surface of the sleeve body 502. In the embodiment of FIG. 5, the sheath 530 is covering and/or everted over an outer portion of the sleeve body 502, the first expandable sealing mechanism 508, the second expandable sealing mechanism 510, and the roughening material 520 of the anchoring system 500. A portion of the sheath 530 also extends through the inner lumen 503 of the sleeve body 502 to thereby attach to the cylindrical slider element 535 that is disposed within the cylindrical handle 505. In some embodiments, the section of the sheath disposed within the inner lumen of the anchoring device may comprise woven fibers, one or more discrete filaments, a flexible cylindrical tube structure, or a combination thereof.

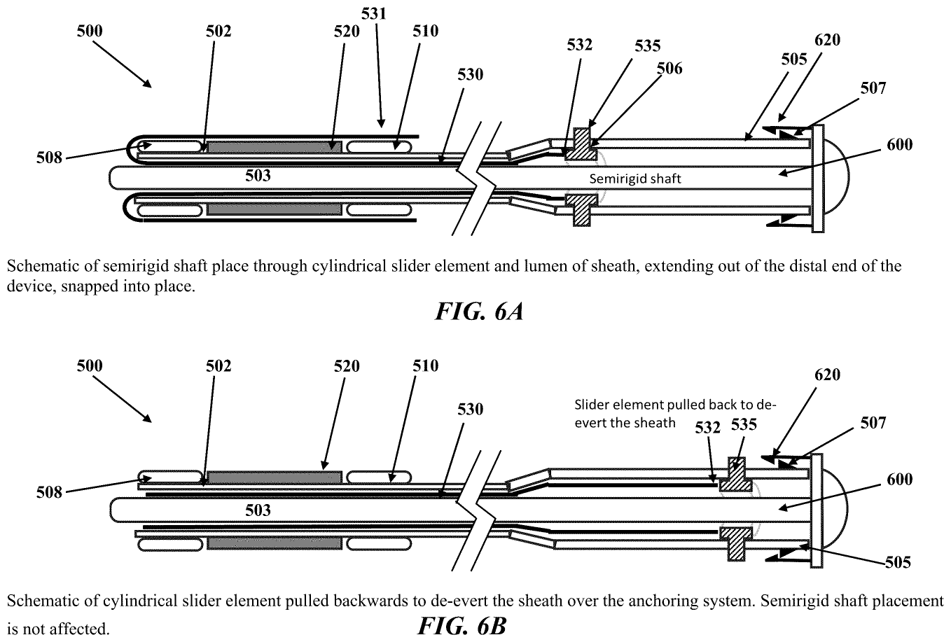

In some embodiments, the sheath and slider element are joined mechanically together such that they create one continuous luminal path to the opposite end of the device. The semi-rigid shaft would then be placed through this luminal path, and in one embodiment, secured into place mechanically at the end of the handle element. In one embodiment, this securing is done by a snap fitting. The semi-rigid shaft that is provided with the device may have this snap fit feature built into its end. This configuration allows for removal of the semi-rigid shaft, while leaving the smooth sheath in the implantable portion of the device for easy exchange with an endoscope. For example, FIG. 6A provides an illustration of this embodiment in which the sheath 530 of FIG. 5, which has a first end 531 that is covering and/or everted over an outer portion of the sleeve body 502, the first expandable sealing mechanism 508, the second expandable sealing mechanism 510, and the roughening material 520 of the anchoring system 500, and a second end 532, which is attached to a cylindrical slider element 535 that is disposed within a cylindrical handle 505 but extends through a cut-out 506 provided in the cylindrical handle 505, further includes a semi-rigid shaft 600 that is placed through the lumen 503 of the sheath 530 and/or the sleeve body 502, and extends out of a distal end of the anchoring system 500 but snaps into place via a snap feature 620 provided on the semi-rigid shaft 600 and a snap feature 507 provided on the cylindrical handle 505. FIG. 6B illustrates the removal of the sheath 530 from the proximal end of the anchoring system 500 to thereby uncover the outer portion of the sleeve body 502, the first expandable sealing mechanism 508, the second expandable sealing mechanism 510, and the roughening material 520 of the anchoring system 500 that is originally covered by the first end 531 of the sheath 530. By applying tension to the sheath 530 by pulling the cylindrical slider element 535 backwards (i.e., towards to the distal end of the anchoring system 500), the sheath 530 is de-everted and the outer portion of the sleeve body 502, the first expandable sealing mechanism 508, the second expandable sealing mechanism 510, and the roughening material 520 of the anchoring system 500 are uncovered. In this embodiment of FIG. 6B, the semi-rigid shaft 600 remains in place, i.e., its placement is not affected, when the sheath 530 is de-everted by pulling the cylindrical slider element 535 backwards, i.e., toward the engagement of the snap feature 620 provided on the semi-rigid shaft 600 and the snap feature 507 provided on the cylindrical handle 505.

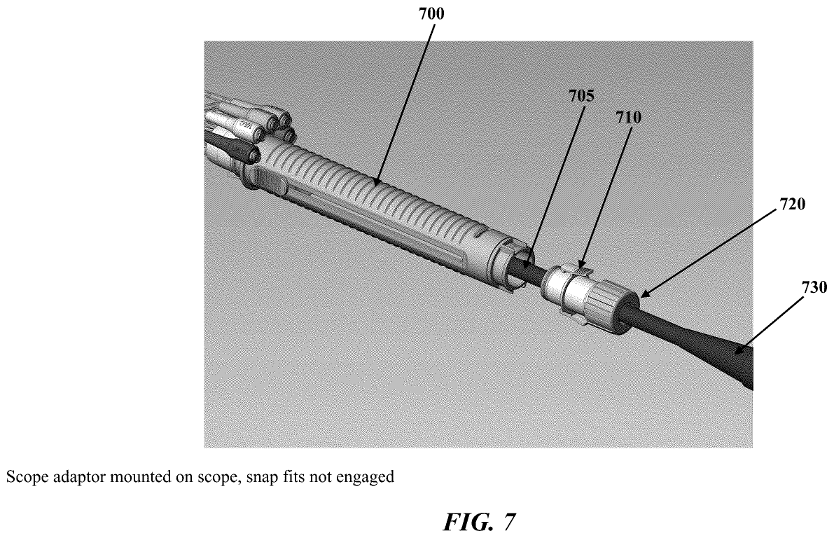

As discussed above, in the configuration in which the semi-rigid shaft is positioned within the lumen of the sheath and/or sleeve body of the anchoring system (see, e.g., FIGS. 6A and 6B), it is possible to remove the semi-rigid shaft, while leaving the smooth sheath in the implantable portion of the device for easy exchange with an endoscope. In this embodiment, an adaptor for an endoscope would be preferred, wherein the adaptor is configured to anchor to the insertion tube of the endoscope. In one embodiment, the anchoring to the insertion tube of the endoscope is accomplished using an adaptor that slips over the insertion tube and then is tightened into place with a twist-to-tighten mechanism, or any other mechanism known in the art. The scope adaptor further would have a means to mechanically connect to the handle described above. In one embodiment, this mechanical connection would be the same snap fit feature that is utilized on the semi-rigid shaft, allowing for easy exchange between the semi-rigid shaft and endoscope with adapter. For example, as shown in FIG. 7, a handle 700 of an anchoring system includes a semi-rigid shaft 705 therein, which engages with an insertion tube 720 of an endoscope 730 via an adaptor 710 as described above. The endoscope 730 would then be inserted through the handle 700, into the luminal path, preferentially such that the tip of the endoscope/colonoscope protrudes out the opposite end of the anchoring device, at which point the scope can be locked into place with the handle 700 using the snap fit feature or adaptor 710. This configuration allows for the advancement and withdrawal of the implantable portion of the anchoring device, sheath, handle, and either the semi-rigid shaft or endoscope with adaptor as a single mechanically connected unit.

In some embodiments, the sheath is also the protective lumen and is not removed. In this configuration, the protective sheath is attached to the anchor portion of the device and is in continuity with the anchor portion of the device. The sheath can be everted over the end of the anchor portion of the device and the sheath is retractable, but stays attached to the anchor to act as the protective lumen for enteric contents to flow through. In these embodiments, the sheath is attached circumferentially to the interior lumen of the device, and then disposed forward such that it extends out the front of the device. It is then everted to cover the anchor section on the outside, doubled over itself, and then de-everted once again at the tip of the device. The sheath then re-enters the lumen of the device and extends through its length. In some of these embodiments, the lumen of the device may not extend beyond the anchor section, and the sheath which stays in place provides the conduit for enteric contents to exit the patient. Prior to activation, applying tension on the sheath would cause the doubled over section extending and covering the outer surface of the anchor to fully de-evert, allowing for anchor activation and/or expansion of the first and second expandable sealing mechanisms.

According to an embodiment, in operation, the device would be advanced through the hollow substantially cylindrical organ cavity (e.g., intestine) to the site where it is desired to be anchored, whether using the provided rigid shaft (see, e.g., FIG. 3) or an endoscope (such as a colonoscope) with an adaptor (see, e.g., FIG. 7). Once positioned, the cylindrical slider element (as discussed above) would be pulled back, which would cause the sheath to retract and expose the anchoring system (see, e.g., FIGS. 6A and 6B). The anchoring system would be activated and/or the first and second expandable sealing mechanisms would be expanded. Afterwards, the handle (as described above) would be un-coupled from the anchoring device, and the sheath as well as the semi-rigid shaft, which are both coupled to the handle, would all be withdrawn from the device. When an endoscope with an adaptor is used (see, e.g., FIG. 7), the handle would be un-coupled from the anchoring device, and the sheath, as well as the endoscope, which are both coupled to the handle, would all be withdrawn from the device. Alternatively, in some embodiments, retraction or removal of the sheath is accomplished by first removing the semi-rigid shaft or endoscope with adaptor and then withdrawing the sheath.

According to some embodiments of the invention, an ICD delivery device is provided that includes a handle, a guide shaft or rigid shaft (see, e.g., FIG. 3), an outer protective sheath, and a sheath pull handle and/or a cylindrical slider element, which allows a physician or surgeon to place the ICD device at the time of surgery, e.g., colon resection surgery. The ICD delivery system facilitates placement of the ICD device by maintaining a small anchor profile during insertion and protecting the expandable sealing mechanisms (e.g., the inflatable elastomeric balloons) from potential damage when inserting the device past a stapled anastomosis. According to an embodiment, the ICD device is inserted, advanced, and positioned with the ICD delivery system transanally. An aqueous-based lubricant such as, e.g., KY-JELLY®, or a dry lubricant is used to facilitate placement. The anchor portion of the ICD device is positioned 5 cm to 10 cm above (more proximal in the GI tract) the area requiring protection from fecal flow (such as, e.g., the anastamosis after rectal cancer surgery). Importantly, the anchor portion of the ICD device is designed to be placed in a healthy bowel and not at the region of damaged bowel where protection is required. After positioning of the anchoring portion, the outer sheath is removed by withdrawing it using a sheath pull handle and/or a cylindrical slider element (see, e.g., FIGS. 6A and 6B. In some embodiments, the withdrawal of the outer sheath of the delivery system occurs through the center of the guide shaft and/or the inner lumen of the device. The device is then ready for deployment after the outer sheath of the delivery system is removed.

According to an embodiment of the invention, the ICD device deployment procedure is accomplished by (i) inflating the distal and proximal expandable sealing mechanisms (e.g., the balloons) by injecting a non-compressible fluid (e.g., normal saline, mineral oil, or dye-based or radiopaque contrast) through a syringe connected to distal and proximal filling ports of the ICD device, respectively, (ii) connecting a negative pressure source (e.g., a vacuum source) to a vacuum port to anchor the ICD device and/or activate negative pressure, and (iii) withdrawing the ICD delivery system handle and/or guide shaft (or rigid shaft) from the ICD device. According to another embodiment, the external portion of the ICD device is further anchored to a patient's skin using an adhesive dressing. In some embodiments, an optional extension tubing can extend the length of tubing to the effluence bag for bedside to gravity use. The ICD device is designed to be easily removed at bedside or in an outpatient clinic without the need for an additional invasive procedure. According to an embodiment, removal of the ICD device is accomplished by (i) disconnecting the negative pressure source (e.g., a vacuum source) and releasing the vacuum, (ii) deflating the distal and proximal expandable sealing mechanisms (e.g., the balloons) by completely removing fluid through a syringe connected to proximal and distal ports of the ICD device, respectively, (iii) injecting a small amount of normal saline through the vacuum port to break the anchoring seal, and (iv) removing the external dressing and gently pulling the ICD device sleeve until the ICD device is fully removed from the anal verge.

Another aspect of the invention is to provide features for clearing blockages within the anchoring device which may accumulate. In the physiological course following resection surgery, intestinal activity is typically minimal for the first few days. As functionality recovers, enteric contents start to form with high liquid content. As time passes, and colonic absorption increase, the enteric contents progress towards a more semi-solid form. This process may be selectively altered, for example, by patient diet or use of medications such as laxatives. Generally, the anchoring device may be removed from the colon prior to a return to normal stool function, while the enteric contents are still substantially liquid and flow well through the lumen of the implantable portion of the device. In some cases, it may be advantageous to assist the motility of enteric contents through the device that may be of higher viscosity or have more solid contents. An irrigation channel is provided in the device that is disposed parallel with the main central lumen. On the exterior end of the device, the irrigation channel is connected to a port, to which a syringe or other infusion device may be attached, to infuse irrigation fluid such as water, saline, mineral oil, or any other fluid known in the art to promote flow of enteric contents. The interior end of the irrigation channel may be open, which provides flow of the infused fluid facing upstream of the device in order to dilute the enteric contents positioned upstream of the device, and promote flow into the device and eventually out of the patient entirely.

Due to the positioning and sizing of the anchoring device, the diameter of the central lumen is generally smaller than the diameter of the intestinal cavity in which it sits. Thus, the flow path of enteric contents encounters a restriction at the entrance of the central lumen of the device. Furthermore, peristaltic contractions of the intestines are not transferred as readily to enteric contents once they are within the device. Therefore, it may be advantageous to provide means to encourage motility of enteric contents not just upstream of the device within the intestines, but also within the lumen of the device itself. In some embodiments, the above-mentioned irrigation channel is disposed co-axially with the central lumen, and there are at least one, and preferably a plurality of openings along the length of the irrigation channel that face into the primary lumen. In some embodiments, these openings are disposed primarily, or solely, at the position of the anchoring system where enteric flow first enters the device, In other embodiments, these openings are disposed along the entire length of the central lumen. When fluid is infused into the irrigation channel, it would then enter into the central lumen at the position of these openings, mixing with the enteric contents, reducing its viscosity and increasing motility. These intraluminal irrigation openings may be coupled to the same channel that opens to the interior end of the implantable portion of the device or they may be coupled to an independent channel running along the length of the device. In another embodiment, these intraluminal channels may be configured to deliver air or fluid to the lumen to “blow-out” obstructions within the channel. In these embodiments, a clip or other means of occlusion could be placed on the outer portion of the device to preferentially displace enteric contents at the inner end of the device.

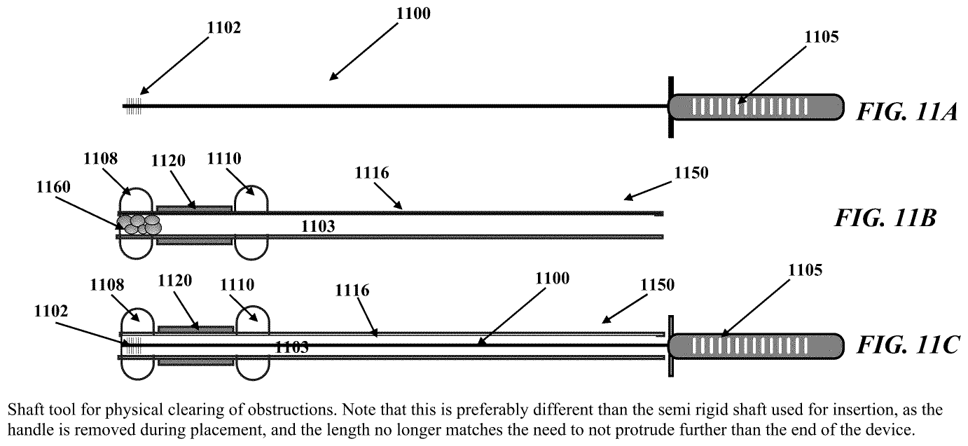

Another aspect of the invention is to provide a means to physically disrupt an impaction within the central lumen of the anchoring device. According to one embodiment, a semi-rigid shaft tool may be provided which is intended to be inserted into the central lumen of the anchoring device from the exterior and reach the entire length of the device within the patient. It is undesirable to have the shaft reach any further than the interior length of the device, as this may cause injury to the hollow organ cavity. Thus, according to one embodiment, the shaft further comprises a stop, which is sized such that it cannot enter the central lumen of the device, and when contact is made with the end of the device and said stop, the semi-rigid shaft tool is positioned such that the tip of the shaft does not protrude past the inner end of the implanted portion of the device. The semi-rigid shaft tool may then be physically manipulated as desired to break up any compactions. FIGS. 11A-11C illustrate one embodiment of this semi-rigid shaft tool that can be used to physically disrupt an impaction within the central lumen of the anchoring device. For example, FIG. 11A illustrates an embodiment of the semi-rigid shaft tool 1100, which includes a handle 1105 and a clearing mechanism 1102 to remove an obstruction. FIG. 11B illustrates an anchoring system 1150 that includes a sleeve 1116 having a central or inner lumen 1103, a first expandable sealing mechanism 1108, a second expandable sealing mechanism 1110, and a roughening material 1120 provided between the first and second expandable sealing mechanisms 1108, 1110. As further shown in FIG. 11B, an impaction 1160 is included within the central or inner lumen 1103 of the sleeve 1116 at the distal end of the sleeve 1116. Thus, as shown in FIG. 11C, the semi-rigid shaft tool 1100 is positioned within the central or inner lumen 1103 of the sleeve 1116 of the anchoring system 1150 in order to remove the impaction 1160 via clearing mechanism 1102. According to some embodiments, the shaft tip and/or body may comprise tines, brushes, fingers, protrusions and/or any other structure or clearing mechanism 1102 known in the art to aid in clearing physical obstructions. The shaft tool may further comprise one or more irrigation channels which may be used to aid in clearance via infusion of fluid from outside the body. According to an embodiment, the semi-rigid shaft tool (see, e.g., FIGS. 11A-11C) is different from the above-discussed semi-rigid shaft used for insertion of the anchoring device, as the handle of the above-discussed semi-rigid shaft is removed during placement, and the length no longer matches the need to not protrude further than the end of the device.

According to some embodiments, the device includes a pneumatic system for applying negative pressure to the anchor system. The device in some embodiments includes an external effluence bag to collect GI content that flows through the sleeve. By providing an external effluence (or collection) bag, the sleeve remains open throughout its length, which allows for constant decompression of the bowel and a continuous open pathway for enteric flow.

However, an external effluence bag is not required for the device to function. In some embodiments, the device has a sleeve that is open just external to the anal sphincter and feces can be passed through this opening. In this embodiment, the anal sphincter constricts around the sleeve and provides some continence and a collection bag is not required.

In some embodiments, the device is intended to be inserted deep into the colon. The anatomy of the colon is such that there are two flexures dividing the ascending colon: the transverse colon and the descending colon. In the case that the device is to be inserted past at least one of these flexures, it is advantageous to have the sleeve containing the central lumen of the device be able to bend around these flexures without kinking or bending in a way that obstructs flow. In some embodiments, the sleeve comprises additional semi-rigid supports built into the sleeve wall to allow it to bend. Such structures may comprise corrugations, braided supports, spiral wireform supports, or other supports, which enable flexibility but prevent kinking known in the art.

According to some embodiments, the ICD device contains at least two additional features. According to one embodiment, an irrigation lumen is provided that is used (if necessary) to instill normal saline proximal to the anchor portion of the device to liquify formed stool and maintain patency of the diversion sleeve and/or sleeve body. In some embodiments, this irrigation lumen is in communication with the proximal in bowel opening of the sleeve and/or sleeve body. According to another embodiment, a contrast lumen is provided that can be used to inject radiopaque dye distal to the anchor portion of the device within the bowel to perform a radiographic leak test.

According to an embodiment, the ICD system is used with a specialized adhesive dressing, a negative pressure source, and a collection bag. These features may be accessory products to the main system. In addition, according to one embodiment, the ICD system includes a scope adapter that allows for placement of the ICD device within the intestine through the use of an endoscope or colonoscope for direct visualization for guided insertion.

In accordance with the features of the embodiments of the invention, when the ICD device is loaded on the delivery system or on an endoscope, sigmoidoscope, or colonoscope, the ability of the ICD device anchor section to move forward against an external resistance is due to the combined effect of (i) the column strength for the size, shape, and material type of the outer sleeve body and/or the sleeve of the anchoring device, and (ii) the internal support (e.g., guide shaft or rigid shaft of the delivery system) provided by the size, shape, and material type for the internal supporting member. The internal support member might include the shaft of a colonoscope or other endoscope or the guide shaft of the ICD delivery system. The unique characteristic of the sleeve and/or the sleeve body allows for a column strength that supports the anchoring system during advancement into the bowel, which allows for the delivery system to function. Moreover, the column strength of the sleeve body and/or the sleeve allows for the external fixation of the ICD device in place. According to one embodiment, the relevant yield strength for column buckling is the compressive yield strength of the chosen material. The column strength of the outer sleeve can be calculated and tailored to the application by proper selection of the material type and material properties. In addition to the column strength of the outer sleeve, the internal support (e.g., guide shaft or rigid shaft of the delivery system) prevents collapse and bucking both axially and laterally. Moreover, the added support allows for the use of a softer and/or thinner wall material, while providing the strength needed to overcome the forward resistance needed to insert the device.

There are several key distinctions of this bowel protection device from negative pressure wound therapy treatment devices that may be used within the intestine. The disclosed anchoring portion of the device is not configured to treat an area of bowel injury, wound, or anastomosis directly. It is configured for anchoring a sleeve portion of the device that protects the area of bowel injury, wound, or anastomosis. Importantly, the anchor portion of the device is designed to be positioned in healthy uninjured bowel above or proximal in the bowel from the area of bowel injury. This method dramatically increases the potential safety of this device as negative pressure is not delivered to the area of the anastomosis, damage or injury; thus, the protected area of bowel is never made ischemic or exposed to significant shear or traction forces from the device.

Negative pressure when delivered through a sponge interface to tissues has been shown to reduce the blood flow to areas where it is delivered. Thus, delivering negative pressure to the damaged area of the bowel itself can further damage the bowel or prevent healing as the blood supply of the bowel is less robust than for other tissues (especially at an area of anastomosis). Furthermore, the method and device described has a flexible sleeve that covers the area of bowel anastomosis or damage; thus, the anchoring of the device is in a separate location than the area of damaged tissue. During bowel contraction at the area of damaged bowel, there are less mechanical forces exerted on the bowel when it constricts around the device because the flexible sleeve is less mechanically rigid than a negative pressure wound therapy dressing that employ wire-stent based internal structures to maintain luminal patency and facilitate anchoring. In addition, by placing the anchor far away from the area of damaged bowel, the device does not exert mechanical force on the anastomosis or damaged tissue with traction or pulling on the device from the pressure tubing or other portions of the device that are external to the patient's body. No portion of the device is anchored distally to the damaged bowel; thus traction is only exerted on the proximal healthy bowel tissues. This further diminishes the risk of pulling apart an anastomosis repair or further injuring an area of damaged bowel.

Another difference is that the anchoring device described herein must have a much higher pullout strength as it must anchor strongly enough to maintain the entire sleeve and anchor element in position in normally functioning, uninjured bowel. To accomplish this, the anchor has to be made long enough and wide enough to allow for adequate surface area of sponge contact to prevent expulsion, the anchor and sleeve must be configured to conform to resist displacement by peristalsis, and the expandable sealing mechanism must be made more robust to prevent potential air leaks.

Unlike a device that is designed to be mechanically dislodged by bowel function and peristalsis over time, the described device is designed to stay in place over an extended period of time until it is removed by the treating clinician. The higher anchoring strength of this anchoring system 100 and more solid fixation is important because it allows for placement of the device near the site of bowel being treated. In cases of bowel anastomosis in the colon, placement of a device higher into the bowel from the anus becomes more challenging due to the curvature of the bowel. So unlike devices that must be placed much higher (>40 cm above area to be treated) in the bowel due to device migration during the treatment period, the fixed anchoring provided by the disclosed anchoring system enables the anchoring element (sleeve, expandable sealing mechanisms, and roughening material (e.g., foam)) to be placed only a couple of centimeters above the area to be treated. However, it may be preferable to have the anchor element placed at least 10 cm above the area to be treated to avoid any potential further damage to the vulnerable segment of bowel.

This ability to deliver controlled anchoring is achieved through the described device elements elaborated on below.

The components of the anchoring system according to some embodiments of the invention are described in detail below. Reference is made to the figures as discussed above unless indicated otherwise.

### Sleeve Body

According to some embodiments of the invention, the sleeve body 102 is a flexible, concentric tube. In some embodiments, the sleeve body 102 is attached and/or bonded to the outer surface of sleeve 116 and provides localized support in the anchor section while allowing more softness and flexibility along the full length of sleeve 116. In some embodiments, the sleeve body 102 provides localized added support to prevent collapse of the sleeve 116 and the inner lumen under expansion of the expandable sealing mechanisms 108 and 110 and added support to prevent angular displacement of the expandable sealing mechanisms 108 and 110 after expansion. The sleeve body 102 can be tailored to provide higher or lower strength by varying wall thickness and material hardness as described below.

The outer diameter and profile can be configured to move within the bowel without significant resistance when negative pressure is not being applied to the outer surface of the sleeve body 102. In some embodiments, the external diameter of the sleeve body 102 is between 11 mm and 61 mm in cross-sectional external diameter. The internal diameter of the sleeve body 102 determines the diameter of the inner lumen, and in some embodiments, the sleeve body 102 has an internal lumen diameter of between 10 mm and 60 mm in cross-sectional internal diameter. For anchoring in other tissue cavities than bowel, these parameters will differ based on the hollow viscus in which anchoring is to be achieved. In some embodiments, the sleeve body 102 may have a diameter that is greater than or equal to the diameter of the tissue cavity. In some embodiments, the sleeve 116 may have a diameter that is less than the diameter of the tissue cavity. In some embodiments, the sleeve 116 may have a diameter that is less than 95% of the diameter of the tissue cavity. In some embodiments, the sleeve 116 may have a diameter that is less than 50% of the diameter of the tissue cavity. In some embodiments, the sleeve 116 may have a diameter that is less than 25% of the diameter of the tissue cavity.

In some embodiments, the sleeve body 102 is configured to be flexible enough to be easily removed by pulling on the sleeve 116 to slide the sleeve body 102 out through the bowel and anus, but rigid enough to hold a concentric shape so that it forms a lumen when negative pressure is applied. This allows for easy placement and removal of the device 100. When negative pressure is applied to the outer surface of the sleeve body 102, the sleeve body 102 and roughening material 120 (e.g., foam) surrounding the sleeve body 102 conform to the contours of the GI tract.

The sleeve body 102 is configured to be soft and pliable, and not to cause erosion into the bowel. The sleeve body 102 has enough flexibility and compliance to allow for the proximal and distal ends of the sleeve body 102 to conform to the bowel contours so that the foam-to-bowel wall contact can be maintained during peristalsis and the expandable sealing mechanisms 108, 110 can create and maintain a seal, yet keep the concentric tubular shape of the internal lumen patent so GI contents can pass through. According to some embodiments, the sleeve body 102 comprises medical grade silicone, polyurethane, thermoplastic elastomer, rubber, or other polymer exhibiting the flexibility and rigidity properties described herein. The flexibility of the sleeve body 102 allows it to safely anchor in a patient's body because the flexibility of the sleeve body 102 reduces pressure points created from bowel contraction forces. The sleeve body 102 according to some embodiments has a Shore A hardness between about 20A and about 70A to allow for maximum flexibility while maintaining a concentric form and patent lumen. The sleeve body flexibility is also determined by the body wall thickness. The sleeve body 102 is thin walled, again allowing for deformational forces to act upon it from bowel peristalsis. In some embodiments, the sleeve body 102 has a main body thickness of between 0.1 mm and 8 mm. The thinness allows for more durable materials to be utilized while continuing to accommodate peristaltic motion of the bowel wall.

The flexibility of the sleeve body 102 allows the sleeve body 102 to deform with the bowel during peristaltic motion. Peristaltic motion moves contents within the bowel by sequentially compressing the proximal section of bowel. Because the device is flexible, it maintains a seal between the expandable sealing mechanisms 108, 110, even with deformation by peristalsis or passage of enteric matter. It maintains surface contact between the roughening material 120 (e.g., foam) and the bowel wall, and the bowel is able to compress the device without the device exerting large and potentially damaging forces on the bowel wall in return. With constant negative pressure maintenance, the negative pressure between the expandable sealing mechanisms 108, 110 creates nearer to constant normal forces during peristalsis along the length of the anchor element that prevents migration by maintaining the foam-to-bowel wall relationship. Accordingly, the device partially conforms and moves in conjunction with the bowel wall because of the distribution of the adhesive forces over the entire surface of the sleeve body 102 covered by the roughening material 120 (e.g., foam interface).

The flexibility further allows for the sleeve body 102 to maintain the position of the roughening material 120 (e.g., foam) on the bowel wall without creating shear forces between the roughening material 120 and bowel wall during bowel contractions. When the bowel contracts, the flexible sleeve body 102 deforms with the forces exerted through the attached roughening material 120 so the roughening material 120 can more easily deform with the bowel wall instead of shearing off the bowel wall resulting in device migration.

Furthermore, for a more flexible anchoring element, the peristaltic wave has less ability to push against the anchoring element due to the flexibility and conformity to the contraction. In cases of a more rigid and less conforming body such as a wire-based stent, the peristaltic wave has the resistance of the less deformable body to push against, resulting in device displacement.

Moreover, the flexibility, compressibility, and compliance of the disclosed device aids in placement and removal of the device through the curvature of the bowel lumen. The flexibility allows delivery of the device higher up in the digestive tract as the bowel becomes more tortuous and curved and allows for easy removal. This flexibility also allows for a longer sleeve body 102 that has a larger roughening material 120 surface area and higher resultant anchoring strength to be manipulated into the bowel. This flexibility is also important for the anchoring system 100 as the roughening material 120 itself has a higher friction co-efficient than that of devices without roughening material.

In contrast to the disclosed invention, stent-like devices have a rigidity that resists compression. This rigidity increases the normal forces on the stent and the bowel as the bowel compresses, and causes the stent to slip along the surface of the bowel along with the peristaltic wave of normal bowel contraction. Some stent-based designs do have some compressibility and flexibility, but it is much lower than that of the disclosed device. Because of the low durometer construction, thinness, compressibility, and conformability of the device sleeve body 102 according to some embodiments, the anchor element is much more resistant to displacement by peristaltic activity. The flexibility of the sleeve body 102 and the expandable sealing mechanisms 108, 110 has the further advantage compared to more rigid devices of being more easily maneuvered for placement within the bowel along the normal longitudinal curvature of the bowel lumen. Moreover, when the device is placed in a region of bowel with longitudinal curvature, it is more easily able to conform to accommodate this curvature to maintain roughening material (e.g., foam)-to-bowel surface area contact when negative pressure is applied and prevent pressure points that can potentially damage the bowel wall. In addition, the elimination of a wire-stent based structure greatly enhances manufacturability from both an ease and expense perspective.

The sleeve body length determines the length of the anchoring element, and the length of the anchor portion of the device is also an important characteristic of the device. The anchoring strength of the anchor portion of the device is directly dependent on the length of the sleeve body 102 and associated surface area of the roughening material 120 (e.g., foam) in contact with the bowel wall. Just like the diameter affects the surface area of the roughening material contact, so too does the length of the anchor portion. Unlike a stent, negative pressure dressing, or sheath that is located distally in the colon near the anal verge over an anastomotic site or area of damaged bowel that can be supported in place by the device rigidity and does not need to conform significantly to the bends of the intestine proximally in the bowel, the anchor portion of the system according to some embodiments is constructed in a window of lengths from greater than 3 cm to less than 25 cm in length. Our testing in the porcine model indicates that if the anchor device is less than 3 cm in length with a diameter of 33 mm, it will not have the surface area to maintain a pull-out strength of greater than 5 lbs and may be susceptible to loss of seal and low force device displacement (<5 lbs force). Furthermore, if the anchor portion of the device is longer than 25 cm in length, the device cannot easily be place around the anatomic bends of the intestine and positioned in the intended area of anchoring that is above (proximal in the bowel) the level of the area of bowel to be protected. For applications in other tissue cavities that require less pull out strength, such as a duct or esophagus, for example, the device may be shorter than 3 cm in length. Further, the embodiments of the invention are not limited to a flexible sleeve, and a stent-like sleeve surrounded in foam (or other roughening material) may also be used.

### Expandable Sealing Mechanisms

The device 100 includes expandable sealing mechanisms 108, 110 disposed at each end of the sleeve body 102 and/or along a proximal end of the sleeve 116. According to an embodiment, the expandable sealing mechanisms 108, 110 comprise two soft, inflatable elastomeric balloons that provide atraumatic sealing surfaces to both facilitate negative pressure anchoring and provide a fluid/air tight seal against fecal contamination distal to the anchoring system 100. According to one embodiment, the expandable sealing mechanisms 108, 110 are expanded by injecting a fluid or inflation media (e.g., normal saline, mineral oil, and/or a dye-based (e.g., iodine-based) contrast solution) through a syringe connected to distal and/or proximal ports of the anchoring system 100. The expandable sealing mechanisms 108, 110 contact the inner surface of the tissue cavity in which the sleeve body 102 and/or the sleeve 116 is inserted. The expandable sealing mechanisms 108, 110 serve at least two functions. First, once expanded, they create seals between the proximal and distal ends of the external surface of the sleeve body 102 with the bowel wall to create the negative pressure space where the roughening material 120 (e.g., foam) can suck down to the bowel wall and create anchoring forces. Second, the seals create a fluid- and air-tight seal with the inner surface of the tissue cavity at either end of the sleeve body 102 when the expandable sealing mechanisms 108, 110 are expanded and negative pressure is applied to the outer surface of the sleeve body 102 that diverts GI content through the lumen of the sleeve body 102 and into the same lumen of the sleeve 116 attached to the sleeve body 102. The expansion of the expandable sealing mechanisms 108, 110 minimizes the risk of fecal forward or back flow from causing disruption of the seal as fecal flow is directed towards the central lumen of the sleeve body 102 by the expandable sealing mechanisms 108, 110.

The exact height of each expandable sealing mechanism 108, 110 is less important than the relationship of the sealing mechanisms to the external diameter of the roughening material 120 covering. The expandable sealing mechanisms 108, 110 of the system in some embodiments extend beyond the height of the roughening material 120 at rest so that when expansion and/or negative pressure is applied and the bowel wall collapses, a seal can be formed easily between the expandable sealing mechanisms 108, 110 and bowel wall without interference by the roughening material 120. Thus, the annular diameter of the expandable sealing elements 108, 110 is greater than the annular diameter of the roughening material 120 dispersed on the sleeve body 102 at rest when no expansion and/or negative pressure is applied. In some embodiments, the expandable sealing mechanisms 108, 110 extend at least 1 mm beyond the height of the roughening material 120 at rest.

The expandable sealing mechanisms 108, 110 are made of a soft, inflatable, and flexible material that allows them to be expanded and to conform to the surface of the bowel. This is important because the peristaltic forces of bowel contraction can cause potentially harmful pressure points without this flexibility. For example, the expandable sealing mechanisms 108, 110 can comprise thermoplastic elastomer, silicone, polyurethane, rubber, or other rubber-like materials or polymers. The Shore A hardness of the material can range between about 20A and about 70A. Similar to the low durometer of the sleeve body 102, the low durometer of the expandable sealing mechanisms 108, 110 allows for compression and conformation to the bowel lumen during the sealing process when expansion and negative pressure is applied and during bowel peristalsis. The conformability, flexibility, and compressibility of the expandable sealing mechanisms 108, 110 in a similar fashion to the flexibility of the sleeve body allow decreased displacement during peristalsis and easier device placement and removal.

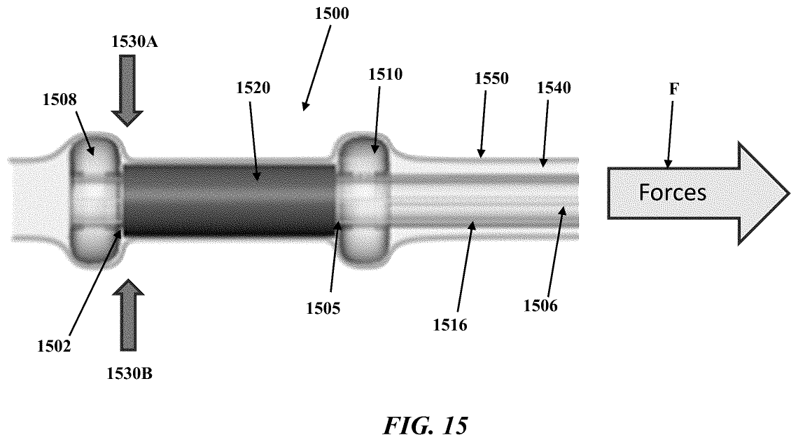

The expandable sealing mechanisms 108, 110 extend radially beyond the external diameter of the sleeve body 102 and/or the sleeve 116 to form seals at each end of the sleeve body 102 and/or along a proximal end of the sleeve 116. The expandable sealing elements 108, 110 extend radially toward the inner surface of the tissue cavity. In some embodiments, the expandable sealing elements 108, 110 extend beyond the roughening material 120 radially, before and/or after expansion, allowing for sealing to occur at the ends of the sleeve body 102 and/or the sleeve 116 without interference from the roughening material 120. In some embodiments, the expandable sealing elements 108, 110 extend or expand to a diameter that is larger than a diameter of the lumen of the tissue cavity (e.g., bowel). (See, e.g., FIG. 15, which is further discussed below). Each expandable sealing mechanism 108, 110 can be a single sealing element or multiple sealing elements. An expandable sealing element is an expandable element that provides for both air and fluid tight sealing and is configured to conform to the GI tissue to create one or more local air and fluid tight seals.

The expandable sealing mechanisms 108, 110 may be configured to be concentrically attached around the outer surface of the sleeve body 102 and/or the sleeve 116 or may be integrated into the wall of the sleeve body 102 and/or the sleeve 116. Specifically, this relates to the manufacturing process used to create the anchor portion, as the expandable sealing elements may be made in one mold with the sleeve body 102 and/or the sleeve 116 or they may be separately molded and adhered to the sleeve body 102 and/or the sleeve 116. According to some embodiments, the expandable sealing elements 108, 110 and sleeve body 102 and/or the sleeve 116 are created as a single molded part as both elements of the device have similar material property requirements of strength, flexibility, and conformability. In some embodiments, the sleeve body 102 and/or the sleeve 116 and the expandable sealing mechanisms 108, 110 are made from a single mold using the same material.

During activation of the anchoring system, in some embodiments, the expandable sealing elements or mechanisms are substantially toroidal balloons positioned circumferentially around the sleeve and/or the sleeve body, as well as the inner or central lumen of the anchoring device. The interior of the balloons is in fluid communication with fill channels, which extend the length of the device to the exterior or proximal end. These fill channels are connected to ports, to which a syringe or other infusion device may be connected, and used to fill the balloons with air or other fluid. In some embodiments, the balloons comprise a semi-porous, thin film material. The porosity of the balloon may be attributable to the balloon material, its thickness or a combination of both. In some embodiments, the fluid used to fill the balloons is substantially non-permeable with respect to the balloon material. In other embodiments, the fill fluid may be a higher viscosity fluid, such as, e.g., dimethylpolysiloxane fluid, or fluorosilicone fluid, or mineral oil. The higher viscosity may be due to longer molecular chains within the fluids which reduce the permeability of the fluid through the balloon materials. Additionally, the higher viscosity fill fluid has high flow resistance through the fill channel, which limits the rate of balloon expansion, which is inherently safer than rapid balloon expansion against an organ wall. The use of a substantially non-permeable filling fluid allows for a thinner balloon construction that benefits this particular anchoring mechanism by both increasing expandability of the balloon mechanisms and lowering the profile of the balloons during placement into the hollow organ body.

According to an embodiment, the fill volume of the balloons may be further controlled using a pressure indicator connected to the infusion device and/or the fill channels. This pressure indicator may be a gauge or threshold-based switch that indicates that the internal pressure of the balloon (from the resistance against the organ wall) is at a level that promotes sealing and does not induce excessive stretch in the organ wall. In some embodiments, the infusion fluid may comprise coloring that may be helpful to indicate if a balloon seal has failed due to rupture or leak. The coloring must be readily discernable and distinctive from other colors that may be present in enteric contents. A coloring pigment that selectively absorbs light wavelengths substantially longer than 650 nm would be preferable, which allows the fill fluid to appear blue. However, other embodiments may use other non-clear coloring that is discernable on physical examination from normal body fluids.

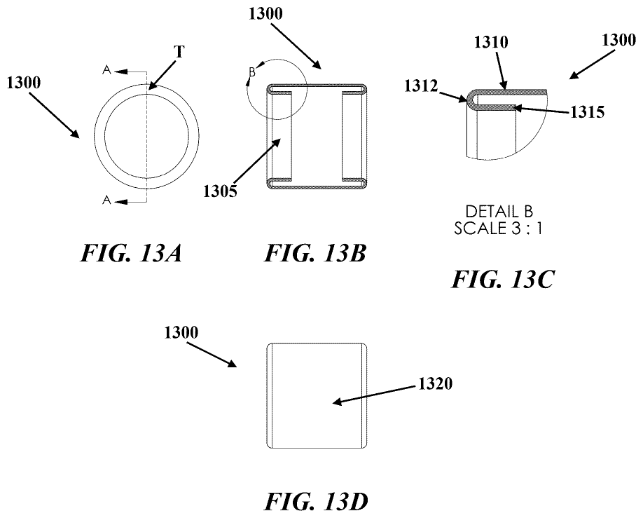

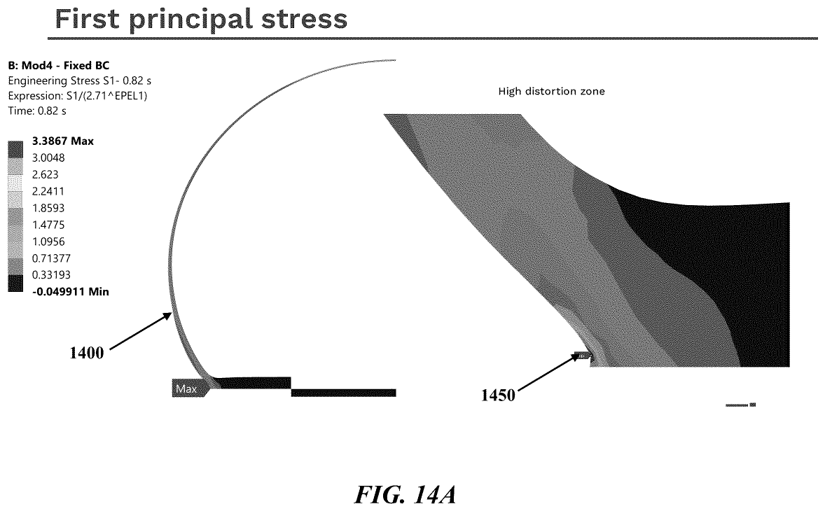

According to one embodiment, the anchoring (or fixation) system depends on having expandable balloon seals in order to function. These seals must be designed in a fashion that allows for collapsing to near flat configuration to allow for easy passage of the device into the bowel (or organ cavity) and expansion to diameters large enough to allow for creation of seals within the bowel and stretching out of some of the bowel's expansion capability. These balloons must further be collapsible so that the device can be easily removed. Furthermore, the balloons must perform reliably and not significantly collapse the smaller inner tubing (or inner lumen) of the pliable main bypass conduit or sleeve when expanded. In order to accomplish these features, the balloons need several important characteristics. First, the balloons need to be flexible enough to allow for the necessary expansion required to go from the nearly flat configuration to the fully expanded configuration to accommodate bowel sizes from 1.5 cm in diameter up to 8 cm in diameter. In order to accomplish this, the wall thickness of the balloons must be in a range of around 0.020 inches to 0.050 inches in thickness (see, e.g., thickness T of balloon 1300 shown in FIG. 13A). Thinner walled material would be susceptible to tearing and rupture and thicker walled material for the balloons would result in poor expansion properties and increased expansion pressure requirements. In addition, the overall durometer and material properties of the balloons that was found to be optimal is between 20-60 Shore A hardness, with a preferred durometer of 30-40 Shore A hardness. This durometer of the material allows for expansion of the material without the necessity of high pressure, but also has the durability to withstand the deforming forces present within the bowel lumens. The durometer also allows for sufficiently thin-walled balloons that allow for lower pressure filling as noted above. Low pressure filling allows the balloons to be compressible and malleable to conform to the shape of the bowel lumen. Lower durometer materials are not strong enough to withstand the expansion and the stress/strain forces within the bowel environment without risk of failure, and higher durometer material have higher filling pressure and stress/strain forces that make the balloons less compressible and mailable.

In some embodiments, each of the first and second expandable sealing mechanisms comprises a flat or low-profile balloon. For example, as shown in FIGS. 13A-13D, a flat or low-profile balloon 1300 is illustrated that includes a wall thickness T as shown in, e.g., FIG. 13A. Thus, such flat or low-profile balloons 1300 have a decreased circumference as compared to traditional expandable balloons. In addition, such flat or low-profile balloons 1300 are easier to put within a tissue cavity or lumen in an unexpanded state and do not create wrinkles in the outer surface of the balloon 1300 when unexpanded or expanded, which thus avoids any issues when contacting the inner surface of the tissue cavity (e.g., bowel). FIG. 13D also illustrates a top view (or width) of the flat or low-profile balloon 1300, which shows how the balloon 1300 has a flattened top 1320.

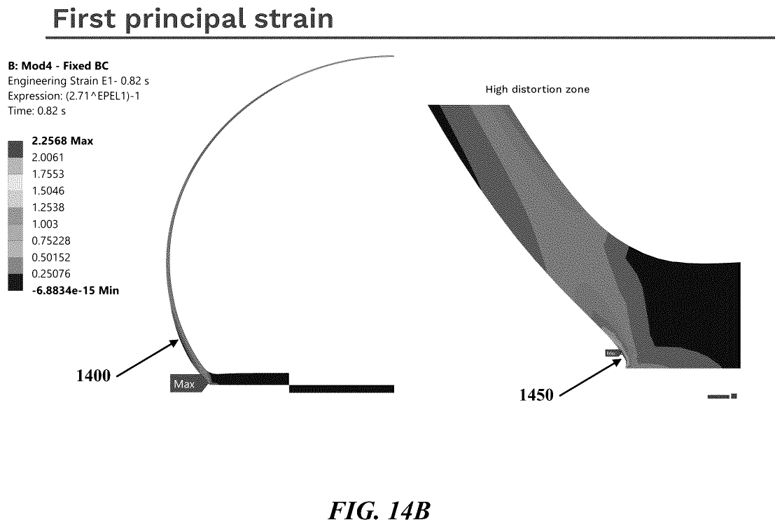

In some embodiments, the first and second expandable sealing mechanisms comprise a balloon having a varying thickness, such that each of the first and second expandable sealing mechanisms has a higher thickness in an area of the respective balloon that attaches or bonds to the sleeve. For example, as shown in FIG. 13B, the flat or low-profile balloon 1300 includes a flap or portion 1305 that is configured to attach to the sleeve and/or sleeve body. However, this flap or portion 1305 of the flat or low-profile balloon 1300 that is configured to attach to the sleeve and/or sleeve body is the portion of the balloon 1300 that encounters the highest amount of stress and strain levels during expansion (see, e.g., FIGS. 14A and 14B, as further described below). Thus, in order to avoid failure of the flat or low-profile balloon 1300, an area of the flap or portion 1305 (i.e., highlighted as area B in FIG. 13B, which is shown in the exploded view of FIG. 13C) is created to have a higher thickness than the rest of the walls of the flat or low-profile balloon 1300. For example, as shown in the embodiment of FIG. 13C, the area B includes a curved area 1312 and a flat area 1315 that are part of the flap or portion 1305 of the balloon 1300 that is configured to attach to the sleeve and/or sleeve body. This curved area 1312 and flat area 1315 of the area B are created with a higher thickness than the rest of the walls of the flat or low-profile balloon 1300, to thereby prevent or minimize the failure of the balloon 1300 during expansion. According to one embodiment, the curved area 1312 and flat area 1315 of the area B are created with a thickness that is from 20% to 30% higher than a thickness of the rest of the walls of the flat or low-profile balloon 1300.

For example, as shown in FIGS. 14A and 14B, a finite element analysis (FEA) was conducted to improve the design and reliability for the balloon construction. Initially, problems were encountered with the high stress and strain levels of the balloon 1400 near the balloon bonding interface 1450 shown in FIGS. 14A and 14B (i.e., the flap or portion 1305 of the flat or low-profile balloon 1300 that is configured to attach to the sleeve and/or sleeve body). Thus, to mitigate the risk of this high stress/strain area, the thickness of this balloon bonding interface 1450 (i.e., area B described above with respect to FIGS. 13B and 13C) was increased to accommodate this area of potential failure. This was done by increasing the thickness in the area (i.e., area B described above with respect to FIGS. 13B and 13C) from 0.020 inches to 0.30 inches, although a thicker amount at the stress area could be additionally beneficial. For example, according to some embodiments, this area has an increased thickness of 20% to 50% thicker as compared to the surrounding areas of the walls of the balloon. By increasing the thickness in this area of weakness (i.e., area B described above with respect to FIGS. 13B and 13C), the reliability and failure of balloons to overexpansion was greatly improved. Thus, this multi-thickness balloon allowed for having an overall thin-walled balloon with the desired expansion and functional properties with improved reliability.

In some embodiments, the expandable sealing mechanisms (see, e.g., expandable sealing mechanisms 108, 110 of FIG. 1A) can be inflated separately and/or independently. In this regard, according to one embodiment, the second expandable sealing mechanism 110 disposed at the distal end of the sleeve body 102 can be inflated first to thereby irrigate, clean and/or flush out any fecal matter around the sleeve body 102 (i.e., the area around the sleeve body 102, the foam 120, and the tissue cavity (or bowel wall)), while the first expandable sealing mechanism 108 disposed at the proximal end of the sleeve body 102 remains flat or uninflated. Such fecal matter disposed around the sleeve body 102 can prevent anchoring of the device within the tissue cavity (or bowel wall) and, thus, flushing out this area of the sleeve body 102 during bowel prep is helpful to assist with anchoring of the device once the negative pressure is applied. This area of the sleeve body 102 can be irrigated, cleaned and/or flushed with, e.g., water and/or saline that is injected into one of the device lumens (e.g., the negative pressure lumen) (see, e.g., flexible tube or tubing 129). After this area of the sleeve body is irrigated, cleaned and/or flushed, the first expandable sealing mechanism 108 disposed at the proximal end of the sleeve body 102 can then be inflated. According to another embodiment, both the first expandable sealing mechanism 108 disposed at the proximal end of the sleeve body 102 and the second expandable sealing mechanism 110 disposed at the distal end of the sleeve body 102 can be inflated at the same time, and then the second expandable sealing mechanism 110 disposed at the distal end of the sleeve body 102 is deflated in order to irrigate, clean and/or flush out any fecal matter around the sleeve body 102, in the same manner as discussed above.

In some embodiments, the sleeve body 102 and/or the sleeve 116 is divided into multiple anchoring portion segments having an independent negative pressure supply. An anchoring portion segment is a section along the sleeve that independently anchors the sleeve. In some embodiments, the sleeve body 102 and/or the sleeve 116 is divided into one or more additional anchoring portion elements to create two or more sealed off areas along the sleeve that independently anchor to the bowel wall. Foam (or other roughening material) is placed between each sealed off section to distribute pressure and interface with the bowel wall. According to some embodiments, the first expandable sealing mechanism 108 and the second expandable sealing mechanism 110 are centralized on the inner lumen of the sleeve body 102 and/or the sleeve 116, which thereby prevents occlusion of the inner lumen of the sleeve body 102 and/or the sleeve 116. Negative pressure is applied to the spaces between the seals, while each expandable sealing mechanism is expanded, to create redundant areas of anchoring along the length of the sleeve body and/or sleeve. In some configurations, negative pressure is applied to each segment from independent negative pressure sources, while each sealing mechanism is expanded from one or more expansion and/or inflation sources. In some configurations, the segments share the same negative pressure source and/or the same inflation source. This embodiment, similar to having multiple anchoring elements, provides redundancy in the anchoring system. The advantage of this design is that if the seal is broken in one segment, there are still adhesive/anchoring forces at another segment or segments.

As discussed, in some embodiments, the expandable sealing elements extend or expand to a diameter that is larger than a diameter of the lumen of the tissue cavity (e.g., bowel). (See, e.g., FIG. 15, which is further discussed below). According to an embodiment, the expandable sealing mechanisms are expanded to create a difference in luminal occupancy that results in a step off between the expandable sealing elements and the inner lumen in an area in which the seal is formed between the expandable sealing mechanisms, the outer surface of the sleeve, and the inner surface of the tissue cavity when negative pressure is applied, such that the inner surface of the tissue cavity (e.g., bowel lumen) conforms to this step off, and thereby displacement forces required to slide the device within the tissue cavity (e.g., bowel) are increased. For example, FIG. 15 illustrates an embodiment in which an anchoring device 1500 is provided within a lumen 1540 of a tissue or organ cavity 1550 (e.g., bowel). As shown in FIG. 15, the anchoring device 1500 includes a sleeve or sleeve body 1502 having an inner surface (not shown) defining an inner lumen 1505. A first expandable sealing mechanism 1508 is disposed at a proximal end of the sleeve body 1502, and a second expandable sealing mechanism 1510 is disposed at a distal end of the sleeve body 1502. A sleeve 1516 is in fluid communication with the sleeve body 1502, and forms a second lumen 1506 that is in continuity with the first (or inner) lumen 1505. A roughening material 1520, such as, e.g., an open-cell foam, is disposed on the outer surface of the sleeve body 1502. The application of negative pressure to the sleeve 1516 and/or the sleeve body 1502 via, e.g., a negative pressure pump, as well as inflation of the first and second expandable sealing mechanisms 1508, 1510, causes a seal to form between the first and second expandable sealing mechanisms 1508, 1510 and an inner surface (or the inner walls) (or lumen) 1540 of the tissue or organ cavity 1550, and creates a frictional force that resists displacement of the sleeve body 1502.

As further shown in the embodiment of FIG. 15, the inflation or expansion of the first and second expandable sealing mechanisms 1508, 1510 creates a difference in luminal occupancy that results in a step off (see, e.g., arrows at 1530A, 1530B) (differential size) between the first and second expandable sealing mechanisms 1508, 1510 and the inner lumen 1505 in an area in which the seal is formed between the first and expandable sealing mechanisms 1508, 1510, the outer surface of the sleeve body 1502, and the inner surface (or lumen) 1540 of the tissue cavity 1550 when negative pressure is applied. As further shown in the embodiment of FIG. 15, the inner surface (or lumen) 1540 of the tissue cavity 1550 (e.g., bowel lumen) conforms to this step off (see, e.g., arrows at 1530A, 1530B), and thereby displacement forces (see, e.g., forces “F” being applied in a direction that is distal to the device) required to slide the device within the tissue cavity (e.g., bowel) 1550 are increased. According to one embodiment, the first expandable sealing mechanism 1508 disposed at the proximal end of the sleeve body 1502 and/or the second expandable sealing mechanism 1510 disposed at the distal end of the sleeve body 1502 are larger (when expanded/inflated) than the inner surface (or lumen) 1540 of the tissue cavity 1550 (e.g., bowel lumen) at the area where the seal is being created, which causes the device to be harder to displace. Moreover, the “step off” (see, e.g., arrows at 1530A, 1530B) between at least the second expandable sealing mechanism 1510 disposed at the distal end of the device and (i) the inner lumen 1505 of the sleeve body 1502 and/or (ii) the inner surface (or lumen) 1540 of the tissue cavity 1550 further makes the device harder to be displaced (as discussed above), as the “step off” (see, e.g., arrows at 1530A, 1530B) becomes a place of fixation. According to one embodiment, this “step off” effect is less pronounced or has less effect at the first expandable sealing mechanism 1508 disposed at the proximal end of the sleeve body 1502. In other words, in the area in which the sleeve body 1502 is positioned within the inner surface (or lumen) 1540 of the tissue cavity 1550 (e.g., bowel), the inner surface (or lumen) 1540 of the tissue cavity 1550 drapes down over the first and second expandable sealing mechanisms 1508, 1510 and the roughening material 1520 (e.g., foam) and creates an acute angle. This acute angle (versus a flat device) makes it harder to displace the device along the tissue cavity 1550 (e.g., bowel) and helps to lock the device in place. This “step off” effect is more effective or pronounced at the second expandable sealing mechanism 1510 disposed at the distal end of the sleeve body 1502. According to an embodiment, the “step off” effect (see, e.g., arrows at 1530A, 1530B) is near perpendicular to the inner surface (or lumen) 1540 of the tissue cavity 1550 that is sucked down when negative pressure is applied (e.g., a bowel “grabbing” point). According to another aspect, the size of the inner surface (or lumen) 1540 of the tissue cavity 1550 is much different when it is sucked down when negative pressure is applied, which thereby creates a size differential between this sucked down, inner surface (or lumen) 1540 of the tissue cavity 1550 as compared to the inflated/expanded first and second expandable sealing mechanisms 1508, 1510, which further results in the inflated/expanded first and second expandable sealing mechanisms 1508, 1510 being less likely to slide down or be displaced along the inner surface (or lumen) 1540 of the tissue cavity 1550 (e.g., bowel).

As discussed above, according to some embodiments, there is less effect with respect to the “step off” effect on the first expandable sealing mechanism 1508 disposed at the proximal end of the sleeve body 1502, which is likely due to less size differential occurring at the proximal end of the device, given that no negative pressure is applied in front of this first expandable sealing mechanism 1508. However, according to some embodiments, it is likely the first expandable sealing mechanism 1508 disposed at the proximal end of the sleeve body 1502 would prevent displacement upstream of the device in the opposite (or proximal) direction. According to some embodiments, the size differential or differences in diameter between the first and second expandable sealing mechanisms 1508, 1510 and the inner lumen 1505 of the anchoring system 1500, as well as the sleeve body 1502 and/or sleeve 1516 allows for better fixation. Such features, including, e.g., the “step off” effect provide another advantage of the balloon design of the instant application. In some embodiments, there are a series (plurality >2) of inflatable sealing elements with negative pressure being applied between these sealing elements to create multiple “step-off” fixation points that when combined further increase fixation and decrease the likelihood of displacement (see, e.g., FIG. 1C).

Moreover, according to an embodiment, the combination of the first and second expandable sealing mechanisms 1508, 1510 and the application of negative pressure to the sleeve 1516 and/or the sleeve body 1502 via, e.g., a negative pressure pump, also prevents a bolus or bulkhead of fluid (e.g., GI contents) traveling through the tissue cavity 1550 (e.g., bowel) to not build up to a point that overcomes the seal created by the anchoring device 1500 with the inner surface (or lumen) 1540 of the tissue cavity 1550. For example, as discussed above, if the inner or central lumen of the sleeve and/or sleeve body is pinched, restricted and/or closed at the distal end of the anchoring device (e.g., near the anal sphincter), any bolus or bulkhead of fluid, air, gas and/or solid fecal matter traveling through the lumen of the device will be pushed back up the device (i.e., toward the proximal end of the device), which can cause failure of the device by, e.g., causing the seal created by the anchoring device with the inner wall of the tissue cavity to fail. Thus, by also providing an anchoring device 1500 having first and second expandable sealing mechanisms 1508, 1510 and a negative pressure system, an improved bypass for enteric flow (e.g., GI contents) is created, which provides a path for any bolus or bulkhead of fluid, air, gas and/or solid fecal matter traveling through the lumen of the device to effectively exit the device. In this regard, according to one embodiment, when a bolus or bulkhead of fluid, air, gas and/or solid fecal matter (e.g., GI contents) with high pressure travels through the tissue cavity 1550 (e.g., bowel), a portion of the bolus or bulkhead of fluid, air, gas and/or solid fecal matter might expand the bowel and get past the second expandable sealing mechanism 1510 disposed at the distal end of the sleeve body 1502. However, the negative pressure system (or pump) quickly evacuates any bolus or bulkhead that passes the second expandable sealing mechanism 1510 disposed at the distal end of the sleeve body 1502, such that no fluid, air, gas and/or solid fecal matter gets past the first expandable sealing mechanism 1508 disposed at the proximal end of the sleeve body 1502. According to this embodiment, even with a bolus or bulkhead of fluid, air, gas and/or solid fecal matter (e.g., GI contents) having high pressure that might expand the bowel wall and/or get past the second expandable sealing mechanism 1510 disposed at the distal end of the sleeve body 1502, the combination of the first and second expandable sealing mechanisms 1508, 1510 and the negative pressure system prevents further fluid, air, gas and/or solid fecal matter from getting downstream to an anastomosis or an area of damaged tissue (or bowel) by evacuating any fluid, air, gas and/or solid fecal matter between the first and second expandable sealing mechanisms 1508, 1510, while the first expandable sealing mechanism 1508 disposed at the proximal end of the sleeve body 1502 also provides a stop gap as the negative pressure system (or pump) sucks out any escaped fluid, air, gas and/or solid fecal matter (i.e., a portion of the bolus or bulkhead of fluid, air, gas and/or solid fecal matter traveling backwards (e.g., proximally) through the device). By contrast, a static system that loses pressure with time and/or that does not include a negative pressure system (e.g., an electric pump) would not be able to easily accommodate this bolus or bulkhead of fluid, air, gas and/or solid fecal matter.

### Sleeve, Extension Tubing, and Collection Bag

The device 100 includes a sleeve 116 that, in some embodiments, is in fluid communication with the sleeve body 102 by being connected to, coextensive with, and/or disposed within the sleeve body 102. According to some embodiments, the sleeve 116 is directly connected to the sleeve body 102. According to some embodiments, the sleeve 116 is indirectly connected to the sleeve body 102. For example, the sleeve 116 may be connected to the second expandable sealing mechanism 110 at the distal end of the sleeve body 102. In some embodiments, the sleeve and sleeve body are a single tubular structure. For example, the sleeve 116 can also be the sleeve body 102 by having the two expandable sealing elements 108, 110 and roughening material 120 (e.g., foam) dispersed on the proximal end of the sleeve 116 without a separate sleeve body. The sleeve 116 shares the same lumen as the sleeve body 102. The expandable sealing mechanisms 108, 110 divert GI content into the sleeve body 102. When the GI content reaches the distal end of the sleeve body 102, it continues through the inner lumen of the sleeve 116. The sleeve 116 can have a length that is sufficient to extend from the distal end of the sleeve body 102 to a patient's anal canal, and outside the patient's body. Thus, once the GI content enters the sleeve body 102, it is directed into the sleeve 116, and is completely isolated from the inner surface of the patent's bowel distal to the sleeve body 102. The sleeve 116 forms a barrier between the GI fecal flow content and the bowel wall, thereby protecting this portion of bowel. To isolate the bowel wall from fecal flow content, the sleeve 116 should be substantially fluid impermeable. Secondarily, the sleeve 116 also mechanically shields the bowel wall from mechanical expansion forces of GI flow contents.

According to some embodiments, the sleeve 116 is bonded to the sleeve body 102 or the distal (second) expandable sealing mechanism 110. The sleeve 116 can have molded fixation attachments that are configured to lock into the sleeve body 102 or the second (distal) sealing mechanism 110. According to some embodiments, the sleeve 116 is made of non-degradable biocompatible materials. For example, the sleeve 116 can be made of silicone, polyurethane, thermoplastic elastomer, rubber, or other polymer, though the embodiments of the invention are not limited to these materials. The sleeve 116 should be substantially impermeable to fluid and bacteria.

The sleeve 116 is configured so that its diameter allows it to dwell within the GI tract without obstructing the flow of GI flow material through it. In some embodiments, the sleeve 116 has a cross-sectional diameter of between about 10 mm and about 60 mm. The sleeve is made of an appropriate material and is thin and compliant enough so that the sleeve 116 is compressible by the bowel wall and does not eliminate the effects of peristaltic motion on fecal flow. Unlike a semi-rigid drainage tube designed primarily to maintain patency and depend on gravity and gastrointestinal flow pressures for movement of GI contents down the tube, the sleeve according to some embodiments is deformable during peristalsis to allow for serial compressions to move GI contents down the sleeve. This allows for placement of the device more proximally in the bowel, as gravity and GI flow pressure is inadequate to move material through a longer length of sleeve because resistance to flow increases with sleeve length. Furthermore, this compliance and associated flexibility allows for navigation around bowel curvatures, improves patient comfort, decreases the chance of bowel wall damage/erosion, and prevents sleeve clogging. Some embodiments of the sleeve 116 have a wall thickness of between about 50 microns and 5 mm. In some embodiments, the length of the sleeve 116 is sufficient for it to extend beyond the GI tract out of the anal canal after device placement. In some embodiments, the sleeve 116 is between about 8 inches and 72 inches in length. In some embodiments, the device is configured so that traction on the sleeve 116 from outside the body can be used to remove the device from the body cavity. The sleeve 116 must be strong enough to withstand longitudinal traction force without tearing of at least 10 lbs of force so that the sleeve can be used to extract the device after treatment is completed. The sleeve 116 in some embodiments is marked with indicators along its length that show the length of sleeve 116 residing inside of the GI tract or tissue cavity after placement in bowel or other tissue cavity. A user can use the indicators to determine whether the anchor section is migrating. The sleeve 116 according to some embodiments has a fixed length. According to some embodiments, the length of the sleeve 116 can be adjusted by cutting the sleeve 116.

According to some embodiments of the invention, a collection bag is disposed at the distal end of the sleeve 116 to manage the enteric (or GI) contents carried from the sleeve 116. By providing a collection bag, the sleeve 116 remains open throughout its length, which allows for constant decompression of the bowel and a continuous open pathway for enteric flow. The collection bag is air-tight and leakproof, and has sufficient volume to accommodate at least one day of use and a provision to empty the contents. The collection bag attaches to the sleeve 116 and is easily replaced using a quick-connect fitting(s). The collection bag is concealable, and for mobile patients, can be worn on the lower extremities of the body when used with, e.g., elastic leg straps, or can be attached to the bedside when used with infirmed patients. The collection bag is made from materials that are resistant to GI contents, provide an odor barrier, and are biocompatible for direct skin contact use. The collection bag is provided non-sterile in a single-use peel open pouch.

### Roughening Material

The device 100 includes roughening material 120 (e.g., foam) that is disposed on the outer surface of the sleeve body 102. The roughening material 120 or foam-like material (e.g., air conducting rough surface material) serves a critical role in both increasing anchoring strength and preventing damage to the bowel. The roughening material 120 provides a critical friction force to hold the sleeve body 102 in place when suction is applied to the outer surface of the sleeve body 102. In addition, the roughening material 120 distributes negative pressure and forces to minimize pressure points that might damage the bowel.

The roughening material 120 dispersed on the sleeve body 102 and/or the sleeve 116 provides a high friction coefficient material with a maximum surface area where adhesion is created by the normal force created with negative pressure. Foam and/or an air conducting rough surface material is an optimal material for distributing negative pressure in this application and providing an effective coefficient of friction when negative pressure is applied. One could envision a device that uses a membrane with a series of holes placed in close proximity to form a porous membrane to distribute negative pressure. However, the normal force generated by a membrane-based device is limited by the open surface area created by the holes. In addition, the porous membrane has a much lower coefficient of friction than the rough surface of the foam and/or the air conducting rough surface material. The foam and/or the air conducting rough surface material also has a larger surface area of effective contact with the bowel due to its open cell structure and multiple pores for distributing negative pressure throughout its substance. To maintain a comparable pullout strength without foam (or an air conducting rough surface material), the magnitude of negative pressure required would have to increase and place significant point stresses on the bowel.

The roughening material 120 (e.g., foam) and/or the air conducting rough surface material comprises a material that is chosen to produce particular compression characteristics and coefficients of friction to prevent migration of the sleeve body 102 and/or the sleeve 116. The roughening material 120 and/or the air conducting rough surface material can comprise a material having a pore size that allows negative pressure to be distributed throughout the roughening material, while preventing ingrowth of tissue into the roughening material. This allows the roughening material 120 and/or the air conducting rough surface material to be easily dislodged from the inner surface of the tissue cavity when normal pressure is restored. In order to have the characteristics required to distribute negative pressure and create a high friction force, some embodiments of the roughening material 120 and/or the air conducting rough surface material have an average foam pore size between about 50 microns to about 1000 microns in diameter. The average pore size of the roughening material 120 and/or the air conducting rough surface material in some embodiments is between about 100 and 300 microns. The average pore size of the roughening material 120 and/or the air conducting rough surface material in some embodiments is between about 200 and 400 microns. The average pore size of the roughening material 120 and/or the air conducting rough surface material in some embodiments is between about 300 and 600 microns. Too small a pore size and the roughening material 120 and/or the air conducting rough surface material loses some of its friction ability and too large a pore size and the material may have tissue ingrowth and has a lower tear strength. In some embodiments, the density and material composition of the roughening material 120 and/or the air conducting rough surface material must allow for an overall tensile strength of the foam to be at least about 50 Kpa. This allows deforming forces and traction on the sleeve body 102 to not shear or tear the roughening material 120 and/or the air conducting rough surface material. Because the roughening material 120 and/or the air conducting rough surface material is bearing the shear force exerted on the anchoring system 100 the roughening material must have a high tear force that can withstand about 50 Kpa of shear force and must be fixed to the sleeve body 102 in a fashion that can withstand about 50 Kpa of distraction force without separation. The level of forces exerted on the device both from the peristaltic and expulsive forces on the sleeve body 102 and sleeve 116 are much higher than for keeping in place the roughening material to treat a small wound area as might be done with negative pressure wound therapy.

The roughening material 120 and/or the air conducting rough surface material in some embodiments is comprised of a material that is hydrophilic, which can prevent the roughening material from drying out the surface tissue with which it comes into contact, though a hydrophobic material can also be used in some embodiments. According to some embodiments, the roughening material 120 and/or the air conducting rough surface material comprises polyvinyl alcohol. In some embodiments, the roughening material 120 and/or the air conducting rough surface material is made of polyurethane, another polymer, or organic fiber mesh. In some embodiments, the roughening material 120 comprises a single tubular piece of foam.

The roughening material 120 and/or the air conducting rough surface material covers the outer surface of the sleeve body 102 and/or the sleeve 116, and creates a friction force when negative pressure is applied to the outer surface that resists motion of the sleeve body 102 and/or the sleeve 116 with respect to the bowel. The porosity of the roughening material 120 and/or the air conducting rough surface material allows air to be evacuated from the region between the outer surface of the sleeve body 102 and the inner surface of the tissue without strong suction being applied to any single point. This creates a frictional force that is evenly distributed across the outer surface of the roughening material 120 and/or the air conducting rough surface material. The roughening material 120 and/or the air conducting rough surface material under negative pressure also creates a large surface area where frictional forces are created to resist dislodgement. The roughening material 120 and/or the air conducting rough surface material is designed to be compressible to minimize the amount of force exerted on any single point of the bowel when negative pressure is applied, and to maximize the surface area contact to the bowel wall by conforming to the shape of the bowel wall.

In some embodiments, the roughening material 120 and/or the air conducting rough surface material dispersed over the sleeve body 102 and/or the sleeve 116 must have a thickness or height that allows for dispersion of negative pressure around the sleeve body 102 and/or the sleeve 116 but does not extend beyond the height of the radial edge of the expandable sealing mechanisms 108, 110 at rest and/or after expansion, or results in narrowing of the sleeve lumen 106 to the point of obstructing GI content flow. If the roughening material 120 and/or the air conducting rough surface material is too thin, it will collapse or clog and not have enough open pores to evenly distribute negative pressure around the sleeve body 102 and/or the sleeve 116. If the roughening material is too thick, it will prevent air tight seals from initiating at the expandable sealing mechanisms 108, 110 and constrict the diameter of the sleeve lumen 106. In some embodiments, the thickness of the roughening material 120 and/or the air conducting rough surface material disposed around the sleeve body 102 and/or the sleeve 116 is between 2 mm and 1.5 cm.

According to some embodiments, the roughening material 120 and/or the air conducting rough surface material can be segmented into separate subunits. In some embodiments, multiple pieces of foam are dispersed around each anchoring segment. As described above, these segments can be separated by multiple serial sealing elements. In these embodiments, negative pressure can be applied to all of the subunits in parallel or separately through independent negative pressure supplies.

Alternatives to foam may be used in some embodiments of the disclosed invention to form the interface with the bowel wall. These foam-like alternatives, which include, e.g., air conducting rough surface material, must distribute negative pressure evenly through the material, create a significant friction force to resist displacement when negative pressure is applied, have biocompatibility with the tissues of the GI tract, and compressibility and deformational properties that resist expulsion and pressure induced tissue damage. Some potential polymer-based alternatives or air conducting rough surface material are stacked mesh matrices that are wrapped around the sleeve or sleeve body, a honey-comb lattice of interconnected channels oriented in a radial fashion around the sleeve or sleeve body, or 3-D woven synthetic fabric material. Natural fiber alternatives include gauze, naturally occurring sponges, or woven fabric. However, some embodiments of this device utilize open-cell reticulated foam. According to one embodiment, the air conducting rough surface material have an average pore size of between about 200 and 400 microns. According to some embodiments, the air conducting rough surface material have a higher coefficient of friction than that of the sleeve 116 and/or the sleeve body 102.

According to some embodiments, the roughening material comprises at least one of a porous fiber or matrix, a polymeric material having small protrusions, an open-cell foam, or a combination thereof.

### Pneumatic System

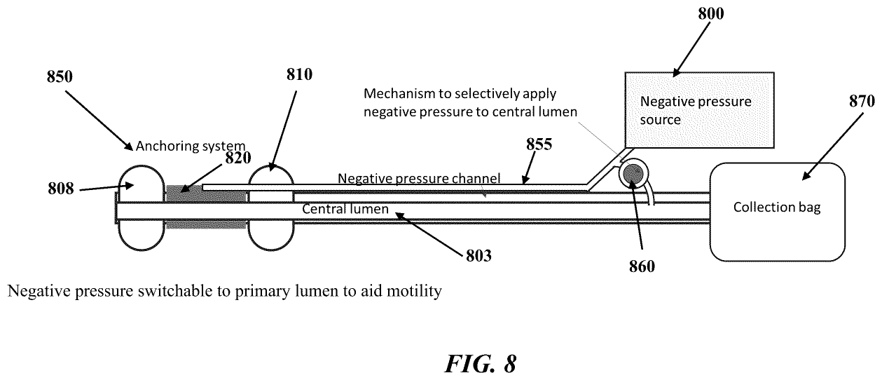

To promote anchoring, a negative pressure source is disposed and typically provides negative pressure to the roughening material of the anchoring system. This negative pressure source may be a spring-powered suction device, a pre-loaded vacuum bottle, a syringe, a powered or electric pump, or a combination thereof. In some embodiments, means may be provided to selectively apply negative pressure from the same source or different source to the central lumen, in order to promote motility of enteric contents down the lumen. In one embodiment, the negative pressure is provided to the anchoring system via a negative pressure channel which is disposed parallel to the primary channel. In these embodiments, the negative pressure channel further comprises a “tee” or “wye” junction which is then selectively in fluid communication with the primary lumen. The selectivity of this connection is preferentially provided by a manual activation means, such as, e.g., a manual switch or a spring-loaded button. A mechanism that by default closes the communication with the primary lumen is preferable. It is also preferable that activation of this mechanism does not alter the delivery of negative pressure to the anchoring system. When this mechanism is activated, the negative pressure is then delivered into the central lumen, preferentially at a substantially downstream location, thus providing additional motility to the enteric contents temporarily. For example, FIG. 8 illustrates an embodiment in which a negative pressure source 800 is provided that is in communication with the inner or central lumen 803, the roughening material 820, and/or the first and second expandable sealing elements 808, 810 of the anchoring system 850 via a negative pressure channel 855. A mechanism 860 is provided in order to selectively apply negative pressure to the central lumen 803 of the anchoring system 850. As further shown in the embodiment of FIG. 8, a collection bag 870 is attached to the proximal end of the anchoring system 850 in order to collect any enteric (GI) contents flowing through the central lumen 803 of the anchoring system 850. In the embodiment of FIG. 8, the negative pressure being supplied by the negative pressure source 800 is switchable to the central (primary) lumen 803 to aid in motility.

According to one embodiment, the device 100 includes a pressure tube (not shown) that is in fluid connection with the outer surface of the sleeve body 102 and/or the sleeve 116. The pressure tube is connected to a negative pressure source such as an air pump, electric pump or vacuum that sucks air out of the tube in a controlled fashion. This pump maintains either constant or variable negative pressure at a level of pressure that allows for adequate anchoring so that the anchor portion does not become dislodged, but does not harm the bowel. The configuration of device allows for physiologically safe pressures of up to −200 mmHg, though pressures of −50 to −150 mmHg may be the preferred range of negative pressure delivery. Negative pressure is applied to the tube, after a seal is formed by expansion of the expandable sealing mechanisms 108, 110 at either end of the sleeve body 102. As the pressure tube connected to a negative pressure source continues to apply negative pressure, the inner walls of the tissue cavity are pulled toward the outer surface of the sleeve body 102 and/or the sleeve 116, bringing the tissue into contact with the roughening material 120. The normal force created by the negative pressure sucking down the tissue against the roughening material 120 creates a friction force that resists motion of the sleeve body 102. The pressure tube is configured to resist occlusion from wall collapse when negative pressure is applied. In some embodiments, there are more than one pressure tube to provide redundancy in case of kinking or clogging of any one pressure tube. In some embodiments with a plurality of pressure tubes, more flexible and compliant tubing material can be utilized due to the redundancy of negative pressure delivery. Each of these pressure tubes are individually in fluid communication with the roughening material 120 to allow for negative pressure delivery. In some embodiments, where there are multiple anchoring portion elements or in cases where there are multiple anchoring portion segments, there may be separate pressure tubes connected to each anchoring portion element or anchoring portion segment. The plurality of pressure tubes can be connected to a single negative pressure source such as a single pump or individually to a plurality of pressure sources such as multiple pumps.

According to an embodiment, the pressure tube (not shown) extends from the sleeve body 102 beyond the anus. The pressure tube can be disposed within the wall of the sleeve 116 or be separate. According to some embodiments, the sleeve 116 defines an additional lumen in which the pressure tube is disposed such that it is isolated from the GI content traveling through the sleeve 116. Alternatively, the pressure tube can be situated alongside the sleeve 116, either attached to the outside of the sleeve 116, inside the sleeve 116, or detached from the sleeve 116. In another embodiment, an additional lumen in the sleeve 116 is the pressure tube.

The proximal end of the pressure tube can be connected to the distal end of the sleeve body 102 or to the second expandable sealing mechanism 110 disposed at the distal end of the sleeve body 102.

In some embodiments, the pressure tube has an adaptor that can be used to attach a syringe so that the pressure tube can be flushed and the roughening material 120 irrigated with fluid. This can be helpful with removal of the device from the bowel wall during the removal procedure or for flushing GI contents away from the foam interface that might clog the pneumatic system.

According to some embodiments, the pressure tube is part of a pneumatic system that controls the pressure on the outer surface of the sleeve body 102 and/or the sleeve 116. The pneumatic system includes a pump (e.g., electric pump) that pulls air out of the pressure tube and maintains near constant and/or variable negative pressure at a set pressure level in the range of −50 mmHg to −200 mmHg or −50 mmHg to −150 mmHg. Our benchtop testing has demonstrated that −50 mmHg negative pressure to the system can produce >10 lbs longitudinal pullout force. The pneumatic pump may also in some configurations be capable of applying positive pressure, for example to assist in removal of the device from the patient's bowel. The pneumatic pump can maintain negative pressure through an electric pump mechanism or mechanical pump mechanism. The pneumatic system may include an indicator that allows the user to determine whether sufficient negative pressure has been achieved and maintained. For example, the pressure gauge can be an indicator that demonstrates that sealing is maintained as suction force is measured within the pneumatic system.

According to an embodiment, the anchoring system works in conjunction with a negative pressure system that is an electrical pump having an indicator. In this regard, the ability of the anchoring system (or ICD System) to fixate in the tissue or organ cavity (e.g., bowel) is dependent on delivery of consistent negative pressure to the fixation portion of the device from an external negative pressure source. The clinical application of negative pressure for this particular use requires several important characteristics and features that can more easily be accomplished with an electrically powered negative pressure source with associated electrically powered functions. Based on benchtop and clinical testing, these specific parameters have been defined to create a unique device that is specific for this application. Although a non-electrically powered negative pressure source can be used, as disclosed above, the use of an electrically powered device with an electrical processor provides distinct advantages for maintaining the function of the anchoring system when in clinical use. Thus, according to an embodiment, an electrically powered negative pressure source is provided for anchoring a suction-based apparatus (e.g., the anchoring system described herein) within a body cavity such as the intestines.

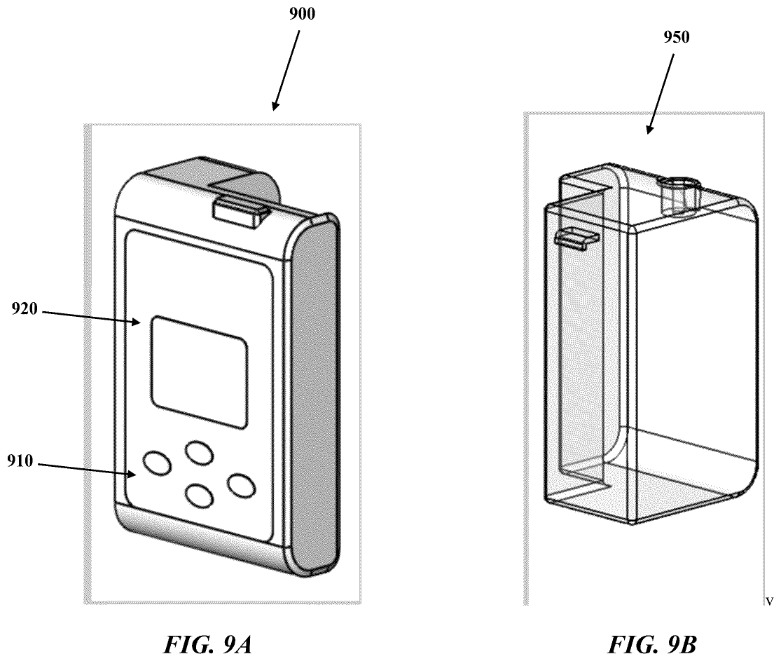

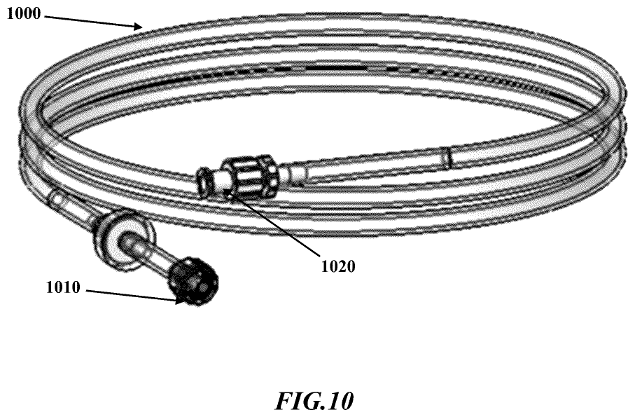

According to one embodiment, the device comprises an electrically powered negative pressure pump attached to a collection canister. For example, FIGS. 9A and 9B illustrate an embodiment of an electrically powered negative pressure pump 900, which can be attached to a collection canister 950. The pump 900 has the ability to deliver continuous negative pressure of up to at least −150 mmHg. The canister 950 comprises a housing 955 that creates an internal chamber 960 configured to hold a volume of fluid, such that the canister 950 has a fluid collection capacity that can hold 100 ml or greater of fluid. The canister 950 further comprises an inlet 970 into which the fluid can enter the internal chamber 960 of the canister 950 via, e.g., a tubing (not shown). The canister 950 also includes a connector member 980 configured to attach the canister 950 to the electrically powered negative pressure pump 900. The pump 900 comprises a housing 905 configured to house electronic controls 910 (e.g., a controller) that allow for the pump to maintain negative pressure in a continuous fashion in a range from around −50 mmHg to −150 mmHg. The housing 905 of the pump 900 is configured to connect to the sleeve of the anchoring device via at least one pressure tube (see, e.g., FIG. 10). The pump 900 has a sensor and/or the ability to indicate that negative pressure has dropped from a pre-determined setting range or threshold (i.e., the negative pressure is not being maintained). This can be called a “low pressure” indicator or alarm 920. In some cases, this range of “low pressure” is less than around −100 mmHg, but in some cases this is less than around −70 mmHg and/or −60 mmHg and/or −50 mmHg (or any other set threshold range or value). The pump 900 can be further configured to indicate if negative pressure is dropping to provide a “leak” indication or alarm. The pump 900 can be further configured to indicate if the device is powered on, but not activated, with an “idle” indication or alarm. The pump 900 in combination with the canister 950 can also be configured to detect if the canister is full and has a “full canister” indicator or alarm. In some cases, this indicator(s) 920 is a display screen. In other embodiments, this indicator(s) is an audible signal. In yet other embodiments, this indicator(s) is an analog indicator. According to some embodiments, the pump 900 includes one or more of (i) a full canister alarm, (ii) a low pressure alarm, (iii) a leak alarm, and (iv) an idle alarm.

The pump 900 can also have a long-lasting battery. The battery should be able to power the device for 24 hours or longer, with the preferred embodiment allowing for functioning of around 3 days or more between charging. The battery allows for increased mobility of the patient during treatment because the patient does not need to be tethered to an electrical wall outlet to power the pump. The pump also can have a “low power” indicator that signals when the battery reserve on the device needs recharging. All of these indicators are important as they provide information to the clinician, caregiver, and/or patient that the anchoring device may not be fixating properly within the bowel. They allow the operator time to correct the problem before the anchoring system becomes displaced. This is critical as the longer that negative pressure is not being delivered to the fixation portion of the device, the more likely the device can become displaced. Thus, timely warning of any problems that might arise significantly mitigates the risk of using the fixation technology.

According to an embodiment, the pressure range was determined based on testing of the anchoring device in a simulated pig intestine model. It was determined that the requirement for fixation based on clinical experience was that retention forces of >10 lbs would be required to keep the device in place. Although tested retention forces were found to be as high as 16 lbs for pressures as low as −40 mmHg, the tear strength of the bowel was often reached (i.e., the test apparatus consisting of a porcine colon would tear apart before displacement). As shown in the data of Table 1 below, fixation in the bowel meets the 10 lb threshold at pressures of −40 mm Hg or greater. Thus, according to one embodiment, the minimum pressure range was set to −50 mmHg.

TABLE 1

| Forces | 20 mmHg | 40 mmHg | 60 mmHg | 80 mmHg | 100 mmHg | 120 mmHg | 150 mmHg |
|---|---|---|---|---|---|---|---|
| Pullout Force | 9 | >10 | >10 | >10 | >10 | >10 | >10 |
| (lb) | | | | | | | |
| Head Pressure | Yes | Yes | No | No | No | No | No |
| Migration | | | | | | | |

As shown in the data of Table 1 above, it was further determined that with simulated boluses of fluid (e.g., head pressure), device migration did not occur unless the pressure dropped below −60 mmHg. Thus, there is an optimal or critical range of anchoring pressure that the device delivers between around −50 mmHg to −150 mmHg for this application. The upper limit of −150 mmHg was determined based on the effects of negative pressure on tissue blood supply. For example, in a patient with a blood pressure in the normal range, an increase in negative pressure (i.e., over −150 mmHg) can cause a decrease to the blood supply. In addition, the literature further supports that higher pressures reduce tissue perfusion. Thus, pressures less than −150 mmHg are deemed safe for human tissues for prolonged use.

Further, it is also known from human studies that the capacity required for exudate during this use case from bowel excretions or any seepage of enteric fluid around the more proximal (in patient) balloon on the anchoring device requires a fluid storage capacity of around 30 ml/day. Thus, for a maximum 14-day period, this would require a canister of 250 ml or more. Thus, a collection canister (see, e.g., canister 950 of FIG. 9B) of between around 50 ml (emptied or replaced daily) to around 500 ml would be ideal for this application, with the preferred embodiment having a 100 ml to 400 ml capacity.

FIG. 10 illustrates one embodiment of a pressure tube 1000 which can be used to connect a negative pressure source (see, e.g., electrically powered negative pressure pump 900 of FIG. 9A) to an anchoring system (as described herein) via a first connection member 1010 and a second connection member 1020. According to one embodiment, the first connection member 1010 and/or the second connection member 1020 of the pressure tube 1000 comprises a one-way valve.

### Insertion and Removal

During insertion into a patient's bowel, the device 100 is introduced into the anal canal and moved past the anastomosis site, so that the second expandable sealing mechanism 110 disposed at the distal end of the sleeve body 102 is proximal to the anastomosis or area to be protected from stool flow. The method of deployment may depend on the level of the anastomosis. For low anastomosis, the device can be deployed through a capsule sheath system that is positioned manually. For higher anastomosis, an endoscope can be used to assist in the deployment. Though use of an endoscope may be useful for both high and low placement within the bowel or passage through an area of bowel damage such as an anastomosis. The device can be placed over the outside of an endoscope and affixed such that a user can position and deploy the device in the desired location.

According to some embodiments, the anchoring system 100 is configured to be placed into position by an endoscope. The device 100 can have a suture or tab present that can be grasped by an endoscope grasper to pull the sleeve body 102 in place using an endoscope. In some embodiments, the device is attached to a releasable clip on the end of the endoscope that can release the device from the end of endoscope from outside the body. Alternatively, an endoscope may be used to hold a flexible member such as a wire or string attached to the anchor that is looped out of the patient's body and pulled around the fixed end of the endoscope within the bowel to pull the device into the bowel and into the desired position. As discussed herein, the device 100 can be connected to an endoscope via a scope adapter (see, e.g., FIG. 7).

In some embodiments, the sleeve 116 can be attached to the endoscope using a releasable mechanism from outside the body clamping mechanism. In some embodiments, the sleeve 116 can be attached to semi-rigid tubing that fits over the endoscope. This tubing is configured to push the anchoring system 100 into place over the endoscope and then to release from the anchoring system 100. In other embodiments, the introducing member is a first semi-rigid tube that contains the proximal portion of device. This first semi-rigid tube is advanced into the bowel through the anus, and after reaching the desired position, a second semi-rigid pushing tube that encircles the sleeve and is smaller in diameter than the first semi-rigid tube is used to hold the device in place while the first semi-rigid tube is removed. The second semi-rigid pushing tube is then removed after negative pressure anchoring of the anchoring system 100 is initiated. See also, e.g., rigid shaft or tube 350 of FIG. 3, which is configured to connect to an endoscope.

According to some embodiments, the ICD device is deployed by (i) expanding the expandable sealing mechanisms 108, 110 (e.g., expandable balloons) by injecting a fluid or inflation media (e.g., normal saline, mineral oil, and/or a dye-based (e.g., iodine-based) contrast solution) through a syringe connected to distal and proximal ports of the device, respectively, (ii) connecting the negative pressure source (e.g., vacuum source or electric pump) to a port (e.g., vacuum port) to anchor the device and/or activate negative pressure, and (iii) withdrawing the delivery system from the ICD device. According to one embodiment, a dye-based (e.g., iodine-based) contrast solution is used to fill the expandable sealing mechanisms 108, 110 (e.g., expandable balloons) as the osmotic gradient helps to maintain the expandable sealing mechanisms 108, 110 (e.g., expandable balloons) in an expanded form better and also allows for radiographic analysis of the anchor location within the body. According to an embodiment, removal of the ICD device is accomplished by (i) disconnecting the negative pressure source (e.g., vacuum source or electric pump) and releasing the negative pressure or vacuum, (ii) collapsing the expandable sealing mechanisms 108, 110 (e.g., expandable balloons) by completely removing fluid through a syringe connected to distal and proximal ports of the device, respectively, (iii) injecting a small amount of normal saline through a port (e.g., vacuum port) to break the anchoring seal, and (iv) removing an external dressing (if necessary) and gently pulling the ICD device sleeve until the ICD device is fully removed from the anal verge.

### ICD Device Column Strength

According to some embodiments of the invention, the ability of the anchoring section of the ICD device to move forward against an external resistance is due to the combined effect of (i) the column strength for the diameter, wall thickness, shape, and material type of the outer sleeve, and (ii) the internal support provided by the diameter, wall thickness, shape, and material type for the internal supporting member (i.e., guide shaft, rigid shaft and/or endoscope). In this case, the relevant yield strength for column buckling is the compressive yield strength of the chosen material. The column strength of the outer sleeve can be calculated and tailored to the application by proper selection of the material type and material properties. In addition to column strength of the outer sleeve, the internal support (guide shaft or rigid shaft) prevents collapse and buckling both axially and laterally. The added support allows use of a softer and/or thinner wall material, while providing the strength needed to overcome the forward resistance needed to insert the device.

According to some embodiments, the outer sleeve (e.g., sleeve 116) has a column strength of 4.0 lbs., when supported by an internal support member. According to another embodiment, the column strength may range from 3.0 lbs. to 6.0 lbs.

According to some embodiments, the outer sleeve (e.g., sleeve 116) has a wall thickness of 0.035 inches, although the wall thickness may range from 0.020 inches to 0.080 inches in thickness. According to one embodiment, the outer sleeve has a hardness of Shore 60A durometer, although the hardness may range from Shore 40A to Shore 90A.

According to some embodiments, the internal support member (guide shaft or rigid shaft) has a diameter of 0.500 inches, although the diameter may range from 0.375 inches to 0.625 inches. According to an embodiment, the internal support member has a wall thickness of 0.06 inches, although the wall thickness may range from 0.020 inches to 0.080 inches in thickness. According to an embodiment, the internal support member material has a stiffness of 1,600 MPa Tensile E modulus, although the stiffness may range from 220 to 2,500 MPa Tensile E modulus.

The embodiments of the invention described herein provide an anchoring device that is designed to have redundant stool bypass, even if negative pressure fails, because the expandable sealing mechanisms (e.g., balloons) still will block most fecal flow and the device will stay in place with the external fixation due to the column strength of the sleeve and/or the sleeve body. In addition, the use of the device on an endoscope requires that the sleeve and/or the sleeve body will not collapse in order for the device to be pushed into the patient's body. Thus, a certain column strength is necessary in order to ensure the delivery system using the scope adapter will work.

### Manufacturing

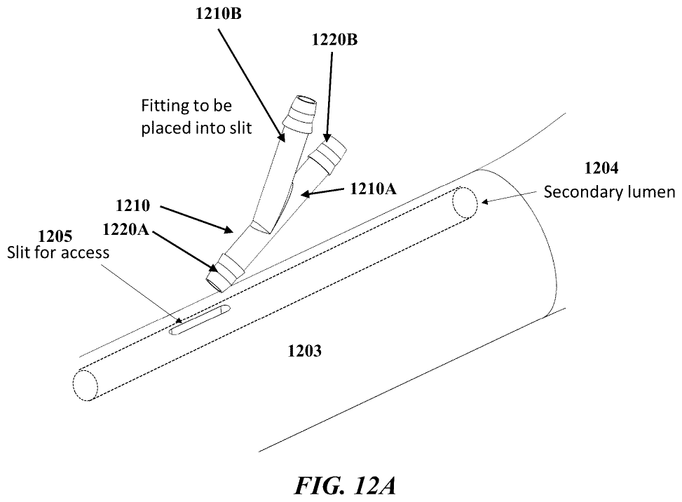

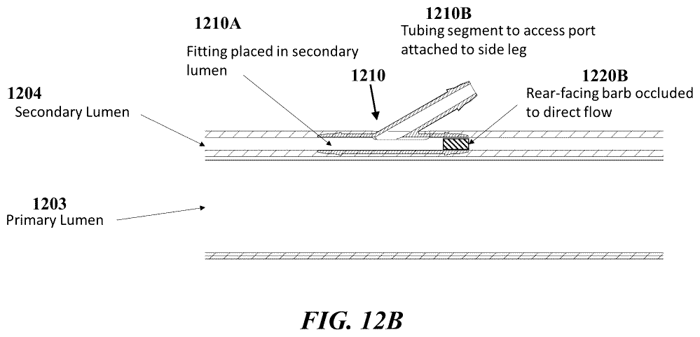

For expediency in manufacturing, in some embodiments, it may be useful to create a central lumen and secondary lumens using a single multi-lumen extrusion. These secondary lumens serve a variety of specific but different purposes, including, e.g., conducting fluid or gas between the access ports and various sections of the device, such as, e.g., irrigation, inflation of the balloons, and/or delivery of negative pressure. In order to access these secondary lumens, according to one embodiment, access ports can be provided that are disposed apart from, but connected to, each individual lumen with a short segment of tubing. This allows the user to have maneuverability and the ability to easily distinguish between the different lumens. Providing a secure, reliable connection into a discrete lumen that is part of a multi-lumen extrusion can be a challenging manufacturing problem. In some embodiments, this connection is provided in the following disclosed manufacturing method and devices. According to this embodiment, which is illustrated in FIGS. 12A and 12B, a small slit 1205 is made in a secondary lumen 1204 provided within the primary or inner lumen 1203 along the axis. A “tee” or “wye” fitting 1210 with a straight-through section 1210A and a side leg section 1210B is then provided, with the side leg section 12010B connected to a segment of tubing and an access port (not shown). The straight-through section 1210A of the fitting 1210 comprises barbs 1220A, 1220B on each end. The slit 1205 in the secondary lumen 1204 is widened, and the fitting 1210 is placed into the slit 1205, such that the straight-through section 1210A now rests in the secondary lumen 1204, positioned axially along its length. Once the front barb 1220A is inserted through the slit 1205, the material is then stretched over the rear barb 1220B to allow it to enter into the secondary lumen 1204. The side leg section 1210B of the fitting 1210 then protrudes out of the slit 1205, providing a connection into the secondary lumen 1204 for the access port (not shown). In some embodiments, one of the ends of the straight-through section 1210A is occluded while the other is patent, the patent segment faces towards the distal end of the device to direct flow in that direction and prevent flow from traveling in the other direction in the secondary lumen 1204.

### OTHER USES

The embodiments of the invention described herein may have uses outside of protection of damaged bowel or anastomosis protection. For example, the disclosed device and method may also be used for continence control in settings like an Intensive Care Unit. In these settings, fecal contamination of the perineum can result in significant skin irritation and breakdown. Existing continence control devices for diverting fecal flow into a collection bag often result in complications such as fecal leaks, displacement of fecal tubes, and erosion into the bowel wall. In contrast, the device and method described here can anchor a fecal collection sheath/sleeve within the rectum of a patient with an anchoring mechanism that is non-traumatic, sealed off from leakage, not easily dislodged, and easily reversible. The anchoring methods described herein may also be used to fixate other sheaths/sleeves or drug delivery devices within the bowel. For example, sheaths/sleeves for limiting absorption used for treating metabolic disorders, diabetes, or obesity may be anchored using the described technique. Specialized sheaths/sleeves designed to elute drugs may also be anchored using the described technique. For example, a sheath/sleeve attached to the anchor device described herein can contain controlled release anti-inflammatory drugs to treat inflammatory bowel disease. Moreover, as described above, a second anchor element can be placed distally to create a sealed space between the treated segment of bowel, the two anchor elements, and the sleeve. This space can be filled with therapeutic solutions such as antibiotics, anti-inflammatory drugs, or chemotherapeutic agents for cancer. This allows for controlled local delivery to a segment of bowel wall isolated between the two anchor elements. Also, as previously mentioned, sleeves may be anchored that may help with diverting flow from a damaged segment of bowel such as a perforation within the bowel, ischemic bowel, bowel contused by blunt trauma, or bowel that is inflamed or dilated such as in cases of inflammatory bowel disease.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

Further aspects of the present disclosure are provided by the subject matter of the following clauses.

An anchoring system comprising a sleeve having an inner surface defining an inner lumen, a first expandable sealing mechanism disposed along a proximal end of the sleeve, a second expandable sealing mechanism disposed along the proximal end of the sleeve, a roughening material disposed on an outer surface of the sleeve, and a sheath covering the outer surface of the sleeve, the roughening material, and the first and second expandable sealing mechanisms, wherein the sheath is configured to be removed via the inner lumen after insertion of the system into a tissue cavity, and wherein expansion of the first and second expandable sealing mechanisms and application of negative pressure to the anchoring system, causes a seal to form between the first and second expandable sealing mechanisms, the outer surface of the sleeve, and an inner surface of a tissue cavity.

The anchoring system according to any preceding clause, wherein the sheath comprises a smooth material.

The anchoring system according to any preceding clause, wherein the roughening material comprises at least one of a porous fiber or matrix, a polymeric material having small protrusions, an open-cell foam, or a combination thereof.

The anchoring system according to any preceding clause, wherein the application of negative pressure brings the roughening material disposed on the outer surface of the sleeve into contact with the inner surface of the tissue cavity thereby creating frictional force that resists displacement of the sleeve.

The anchoring system according to any preceding clause, wherein the first and second expandable sealing mechanisms are expanded by providing a non-compressible fluid to expand the first and second expandable sealing mechanisms.

The anchoring system according to any preceding clause, wherein each of the first and second expandable sealing mechanisms comprises a low-profile balloon.

The anchoring system according to any preceding clause, further comprising (i) a third expandable sealing mechanism disposed along the proximal end of the sleeve, and (ii) a fourth expandable sealing mechanism disposed along the distal end of the sleeve.

The anchoring system according to any preceding clause, wherein the sheath is disposed through the inner lumen and through an entire length of the anchoring system to a region of the anchoring system that is outside of a patient and at an opposite end of the inner lumen where an anchor portion is disposed, such that the sheath everts back onto itself, and extends back down the length of the anchoring system on the outer surface, thereby covering the anchor portion.

The anchoring system according to any preceding clause, wherein the anchor portion includes the first expandable sealing mechanism disposed along a proximal end of the sleeve, the second expandable sealing mechanism disposed along the proximal end of the sleeve and the roughening material positioned between the first and second expandable sealing mechanisms.

The anchoring system according to any preceding clause, wherein the sheath is configured to be removed by applying tension on the sheath at an end of the sheath that is opposite to the anchor portion thereby causing the sheath to de-evert, uncovering the anchor portion, and allowing the sheath to be removed by pulling the sheath through the inner lumen.

The anchoring system according to any preceding clause, wherein a lubricant or other friction-reducing substance is provided along one or more of (i) inner and/or outer surfaces of the sheath, (i) an inner surface of the inner lumen, and (iii) the outer surface of the sleeve.

The anchoring system according to any preceding clause, wherein the lubricant or other friction-reducing substance comprises at least one of (i) a liquid, (ii) a surface coating, (iii) a solid particulate, or (iv) a combination thereof.

The anchoring system according to any preceding clause, wherein the lubricant or other friction-reducing substance are pre-applied to the anchoring system during manufacturing and are already in place when a user is presented with the anchoring system for use.

The anchoring system according to any preceding clause, wherein the lubricant or other friction-reducing substance are provided with a packaging of the anchoring system and a user applies them to the anchoring system prior to use.

The anchoring system according to any preceding clause, wherein a first type of lubricant is provided between the outer surface of the sheath and the inner surface of the lumen and a second type of the lubricant, which is differ from the first type of lubricant, is provided between the inner surface of the sheath and the outer surface of the sleeve.

The anchoring system according to any preceding clause, wherein the anchoring system further includes a semi-rigid shaft element that is disposed within the inner lumen of the sleeve and couples to the anchoring system, such that it may be used to advance the anchoring system to its desired position.

The anchoring system according to any preceding clause, wherein the semi-rigid shaft element attaches to at least one of an endoscope, a colonoscope, or a sigmoidoscope, upon which the shaft element is able to advance the anchoring system into a patient.

The anchoring system according to any preceding clause, wherein the sheath is coupled to a hollow cylindrical slider element, with this slider element disposed within a cylindrical handle.

The anchoring system according to any preceding clause, wherein the cylindrical handle further comprises one or more cutouts disposed axially, wherein a portion of the slider element may protrude through the one or more cutouts.

The anchoring system according to any preceding clause, wherein the sheath and slider element are joined mechanically together such that they create one continuous luminal path to the opposite end of the anchoring system.

The anchoring system according to any preceding clause, further comprising a negative pressure source.

The anchoring system according to any preceding clause, wherein the negative pressure source comprises an electrically powered negative pressure pump attached to a collection canister.

The anchoring system according to any preceding clause, wherein the electrically powered negative pressure pump is configured to provide a continuous negative pressure in a range from around −50 mmHg to −150 mmHg.

The anchoring system according to any preceding clause, wherein the electrically powered negative pressure pump includes a low pressure indicator or alarm.

The anchoring system according to any preceding clause, wherein the electrically powered negative pressure pump includes one or more of (i) a full canister alarm, (ii) a low pressure alarm, (iii) a leak alarm, and (iv) an idle alarm.

The anchoring system according to any preceding clause, wherein the electrically powered negative pressure pump includes a long-lasting battery.

The anchoring system according to any preceding clause, wherein the first and second expandable sealing mechanisms are expanded to create a difference in luminal occupancy that results in a step off between the first and second expandable sealing mechanisms and the inner lumen in an area in which the seal is formed between the first and second expandable sealing mechanisms, the outer surface of the sleeve, and the inner surface of the tissue cavity when negative pressure is applied, such that the inner surface of the tissue cavity conforms to this step off, and thereby displacement forces required to slide the sleeve within the tissue cavity are increased.

A method for anchoring a sleeve in a tissue cavity, with the sleeve having an outer surface comprising an expandable sealing mechanism for isolating a portion of the tissue cavity adjacent to the sleeve from a remainder of the tissue cavity, the method comprising inserting the sleeve in the tissue cavity, expanding the expandable sealing mechanism to create a seal between the expandable sealing mechanism and the inner surface of a tissue cavity, and applying a constant negative pressure in a range of −50 mmHg to −150 mmHg using an electrically powered negative pressure pump to a region between an outer surface of the sleeve and an inner surface of the isolated portion of the tissue cavity to create a frictional force between the outer surface of the sleeve and the inner surface of the tissue cavity.

The method according to any preceding clause, wherein the step of expanding the expandable sealing mechanism is conducted by injecting a non-compressible liquid into the expandable sealing mechanism.

The method according to any preceding clause, wherein the step of expanding the expandable sealing mechanism is conducted by inflating the expandable sealing mechanism.

The method according to any preceding clause, wherein the sleeve further includes a roughening material along the outer surface of the sleeve.

The method according to any preceding clause, wherein the roughening material comprises at least one of a porous fiber or matrix, a polymeric material having small protrusions, an open-cell foam, or a combination thereof.

The method according to any preceding clause, wherein the expandable sealing mechanism comprises a low-profile balloon.

The method according to any preceding clause, wherein the expandable sealing mechanism comprises two or more expandable sealing mechanisms.

The method according to any preceding clause, wherein each of the two or more expandable sealing mechanisms comprises a low-profile balloon.

The method according to any preceding clause, wherein the step of inserting the sleeve in the tissue cavity is conducted using a delivery system that includes a semi-rigid shaft element.

The method according to any preceding clause, wherein the semi-rigid shaft element attaches to at least one of an endoscope, a colonoscope, or a sigmoidoscope, upon which the shaft element is able to advance the sleeve into a patient.

The method according to any preceding clause, wherein the method further includes removing the sleeve from the tissue cavity by (i) releasing the negative pressure, (ii) collapsing the expandable sealing mechanism, and (iii) injecting an amount of saline through an irrigation tubing to break the seal.

The method according to any preceding clause, wherein the applying a constant negative pressure brings the roughening material disposed on the outer surface of the sleeve into contact with the inner wall of the tissue cavity thereby creating frictional force that resists displacement of the sleeve.

The method according to any preceding clause, wherein the expanding of the expandable sealing mechanism creates a difference in luminal occupancy that results in a step off between the expandable sealing mechanism and an inner lumen of the sleeve in an area in which a seal is formed between the expandable sealing mechanism, the outer surface of the sleeve, and the inner wall of the tissue cavity when negative pressure is applied, such that the inner wall of the tissue cavity conforms to this step off, and thereby displacement forces required to slide the sleeve within the tissue cavity are increased.

The method according to any preceding clause, wherein a sheath is disposed through an inner lumen of the sleeve and through an entire length of an anchoring system comprising the sleeve to a region of the anchoring system that is outside of a patient and at an opposite end of the inner lumen where the sleeve is disposed, such that the sheath everts back onto itself, and extends back down the length of the anchoring system on the outer surface of the sleeve, thereby covering the sleeve.

The method according to any preceding clause, wherein the sheath is configured to be removed by applying tension on the sheath at an end of the sheath that is opposite to the sleeve thereby causing the sheath to de-evert, uncovering the sleeve, and allowing the sheath to be removed by pulling the sheath through the inner lumen of the sleeve.

The method according to any preceding clause, wherein the sleeve includes a first expandable sealing mechanism disposed along a proximal end of the sleeve, a second expandable sealing mechanism disposed along a distal end of the sleeve, and a roughening material positioned between the first and second expandable sealing mechanisms.

The method according to any preceding clause, wherein a lubricant or other friction-reducing substance is provided along one or more of (i) inner and/or outer surfaces of the sheath, (i) an inner surface of the inner lumen, and (iii) the outer surface of the sleeve.

The method according to any preceding clause, wherein the lubricant or other friction-reducing substance comprises at least one of (i) a liquid, (ii) a surface coating, (iii) a solid particulate, or (iv) a combination thereof.

The method according to any preceding clause, wherein the lubricant or other friction-reducing substance are pre-applied to the sleeve during manufacturing and are already in place when a user is presented with the sleeve for use.

The method according to any preceding clause, wherein the lubricant or other friction-reducing substance are provided with a packaging of the sleeve and a user applies them to the sleeve prior to use.

The method according to any preceding clause, wherein a first type of lubricant is provided between the outer surface of the sheath and the inner surface of the lumen and a second type of the lubricant, which is differ from the first type of lubricant, is provided between the inner surface of the sheath and the outer surface of the sleeve.

The method according to any preceding clause, wherein the sleeve further includes a semi-rigid shaft element that is disposed within an inner lumen of the sleeve and couples to the sleeve, such that it may be used to advance the sleeve to its desired position.

The method according to any preceding clause, wherein the semi-rigid shaft element attaches to at least one of an endoscope, a colonoscope, or a sigmoidoscope, upon which the shaft element is able to advance the anchoring system into a patient.

The method according to any preceding clause, wherein the sheath is coupled to a hollow cylindrical slider element, with this slider element disposed within a cylindrical handle.

The method according to any preceding clause, wherein the cylindrical handle further comprises one or more cutouts disposed axially, wherein a portion of the slider element may protrude through the one or more cutouts.

The method according to any preceding clause, wherein the sheath and slider element are joined mechanically together such that they create one continuous luminal path to the opposite end of the sleeve.

The method according to any preceding clause, wherein the electrically powered negative pressure pump is attached to a collection canister.

The method according to any preceding clause, wherein the electrically powered negative pressure pump includes a low pressure indicator or alarm.

The method according to any preceding clause, wherein the electrically powered negative pressure pump includes one or more of (i) a full canister alarm, (ii) a low pressure alarm, (iii) a leak alarm, and (iv) an idle alarm.

The method according to any preceding clause, wherein the electrically powered negative pressure pump includes a long-lasting battery.

The method according to any preceding clause, further comprising detecting if the constant negative pressure provided to the sleeve drops below a pre-determined threshold value.

The method according to any preceding clause, wherein the pre-determined threshold value is −70 mmHg.

The method according to any preceding clause, wherein the pre-determined threshold value is −60 mmHg.

The method according to any preceding clause, further comprising emitting an audible signal if the constant negative pressure drops below the pre-determined threshold value.

The method according to any preceding clause, further comprising displaying an indication on a display screen if the constant negative pressure drops below the pre-determined threshold value.

An anchoring device configured to be anchored within a bowel of a patient, the anchoring device comprising a sleeve configured to be positioned within the bowel of the patient, an external portion configured to extend externally from the bowel of the patient, and two redundant methods of anchoring the device within the bowel, wherein a first method of anchoring the device utilizes a negative pressure-based system that comprises an electrically powered negative pressure pump that applies negative pressure to the device to create a frictional force that resists displacement of the sleeve of the device from the bowel, wherein a second method of anchoring the device utilizes first and second expandable sealing mechanisms, wherein each of the first and second expandable sealing mechanisms comprises a low-profile balloon wherein expansion of the first and second expandable sealing mechanisms causes a seal to form between the first and second expandable sealing mechanisms and an inner surface of a tissue cavity, wherein at least one of (i) a column strength of the sleeve is high enough to hold the device in place even if there is failure of the first method of anchoring, (ii) the first method of anchoring is sufficient to hold the device in place even if there is failure of the second method of anchoring, or (iii) the second method of anchoring is sufficient to hold the device in place even if there is failure of the first method of anchoring.

The anchoring device according to any preceding clause, wherein the sleeve further includes a roughening material along an outer surface of the sleeve.

The anchoring device according to any preceding clause, wherein the roughening material comprises at least one of a porous fiber or matrix, a polymeric material having small protrusions, an open-cell foam, or a combination thereof.

The anchoring device according to any preceding clause, wherein the application of negative pressure brings the roughening material disposed on the outer surface of the sleeve into contact with the inner surface of the tissue cavity thereby creating frictional force that resists displacement of the sleeve.

The anchoring device according to any preceding clause, wherein the first and second expandable sealing mechanisms are expanded by providing a non-compressible fluid to expand the first and second expandable sealing mechanisms.

The anchoring device according to any preceding clause, further comprising (i) a third expandable sealing mechanism disposed along the proximal end of the sleeve, and (ii) a fourth expandable sealing mechanism disposed along the distal end of the sleeve.

The anchoring device according to any preceding clause, wherein a sheath is disposed through an inner lumen and through an entire length of the anchoring device to a region of the anchoring device that is outside of a patient and at an opposite end of the inner lumen where an anchor portion is disposed, such that the sheath everts back onto itself, and extends back down the length of the anchoring device on the outer surface, thereby covering the anchor portion.

The anchoring device according to any preceding clause, wherein the anchor portion includes the first expandable sealing mechanism disposed along a proximal end of the sleeve, the second expandable sealing mechanism disposed along the proximal end of the sleeve and a roughening material positioned between the first and second expandable sealing mechanisms.

The anchoring device according to any preceding clause, wherein the sheath is configured to be removed by applying tension on the sheath at an end of the sheath that is opposite to the anchor portion thereby causing the sheath to de-evert, uncovering the anchor portion, and allowing the sheath to be removed by pulling the sheath through the inner lumen.

The anchoring device according to any preceding clause, wherein a lubricant or other friction-reducing substance is provided along one or more of (i) inner and/or outer surfaces of the sheath, (i) an inner surface of the inner lumen, and (iii) the outer surface of the sleeve.

The anchoring device according to any preceding clause, wherein the lubricant or other friction-reducing substance comprises at least one of (i) a liquid, (ii) a surface coating, (iii) a solid particulate, or (iv) a combination thereof.

The anchoring device according to any preceding clause, wherein the lubricant or other friction-reducing substance are pre-applied to the anchoring device during manufacturing and are already in place when a user is presented with the anchoring device for use.

The anchoring device according to any preceding clause, wherein the lubricant or other friction-reducing substance are provided with a packaging of the anchoring device and a user applies them to the anchoring device prior to use.

The anchoring device according to any preceding clause, wherein a first type of lubricant is provided between the outer surface of the sheath and the inner surface of the lumen and a second type of the lubricant, which is differ from the first type of lubricant, is provided between the inner surface of the sheath and the outer surface of the sleeve.

The anchoring device according to any preceding clause, wherein the anchoring system further includes a semi-rigid shaft element that is disposed within the inner lumen of the sleeve and couples to the anchoring device, such that it may be used to advance the anchoring device to its desired position.

The anchoring device according to any preceding clause, wherein the semi-rigid shaft element attaches to at least one of an endoscope, a colonoscope, or a sigmoidoscope, upon which the shaft element is able to advance the anchoring device into a patient.

The anchoring device according to any preceding clause, wherein the sheath is coupled to a hollow cylindrical slider element, with this slider element disposed within a cylindrical handle.

The anchoring device according to any preceding clause, wherein the cylindrical handle further comprises one or more cutouts disposed axially, wherein a portion of the slider element may protrude through the one or more cutouts.

The anchoring device according to any preceding clause, wherein the sheath and slider element are joined mechanically together such that they create one continuous luminal path to the opposite end of the anchoring device.

The anchoring device according to any preceding clause, further comprising a negative pressure source.

The anchoring device according to any preceding clause, wherein the negative pressure source comprises an electrically powered negative pressure pump attached to a collection canister.

The anchoring device according to any preceding clause, wherein the electrically powered negative pressure pump is configured to provide a continuous negative pressure in a range from around −50 mmHg to −150 mmHg.

The anchoring device according to any preceding clause, wherein the electrically powered negative pressure pump includes a low pressure indicator or alarm.

The anchoring device according to any preceding clause, wherein the electrically powered negative pressure pump includes one or more of (i) a full canister alarm, (ii) a low pressure alarm, (iii) a leak alarm, and (iv) an idle alarm.

The anchoring device according to any preceding clause, wherein the electrically powered negative pressure pump includes a long-lasting battery.

The anchoring device according to any preceding clause, wherein the first and second expandable sealing mechanisms are expanded to create a difference in luminal occupancy that results in a step off between the first and second expandable sealing mechanisms and an inner lumen of the sleeve in an area in which the seal is formed between the first and second expandable sealing mechanisms, an outer surface of the sleeve, and the inner surface of the tissue cavity when negative pressure is applied, such that the inner surface of the tissue cavity conforms to this step off, and thereby displacement forces required to slide the sleeve within the tissue cavity are increased.

A electrically powered negative pressure pump for use with an anchoring device configured to be anchored within a tissue cavity of a patient, the anchoring device including a sleeve having an outer surface for contacting an inner wall of the tissue cavity, and an expandable sealing mechanism for isolating a portion of the tissue cavity adjacent to the sleeve from a remainder of the tissue cavity, the electrically powered negative pressure pump comprising (i) at least one pressure tube configured to connect to the sleeve of the anchoring device, to thereby provide negative pressure to the sleeve of the anchoring device to create a frictional force that resists displacement of the sleeve of the anchoring device from the tissue cavity, (ii) a collection canister configured to collect fluid from an area around the tissue cavity in which the anchoring device is anchored, and (iii) a negative pressure indicator configured to detect when the negative pressure provided to the sleeve of the anchoring device drops below a pre-determined threshold value, wherein the electrically powered negative pressure pump is configured to provide a continuous negative pressure to the sleeve of the anchoring device in a range of from −50 mmHg to −150 mmHg.

The electrically powered negative pressure pump according to any preceding clause, wherein the at least one pressure tube is configured to connect to the sleeve of the anchoring device via an one-way valve.

The electrically powered negative pressure pump according to any preceding clause, further comprising a controller configured to maintain the continuous negative pressure to the sleeve of the anchoring device.

The electrically powered negative pressure pump according to any preceding clause, wherein the sleeve further includes a roughening material along an outer surface of the sleeve.

The electrically powered negative pressure pump according to any preceding clause, wherein the roughening material comprises at least one of a porous fiber or matrix, a polymeric material having small protrusions, an open-cell foam, or a combination thereof.

The electrically powered negative pressure pump according to any preceding clause, further comprising one or more of (i) a full canister alarm, (ii) a leak alarm, and (iii) an idle alarm.

The electrically powered negative pressure pump according to any preceding clause, wherein the pre-determined threshold value is −70 mmHg.

The electrically powered negative pressure pump according to any preceding clause, wherein the pre-determined threshold value is −60 mmHg.

The electrically powered negative pressure pump according to any preceding clause, wherein the negative pressure indicator includes an alarm comprising an audible signal.

The electrically powered negative pressure pump according to any preceding clause, wherein the negative pressure indicator comprises a display screen.

The electrically powered negative pressure pump according to any preceding clause, wherein the collection canister comprises a housing that creates an internal chamber configured to hold the fluid collected from an area around the tissue cavity.

The electrically powered negative pressure pump according to any preceding clause, wherein the collection canister further comprises an inlet into which the fluid collected from an area around the tissue cavity enters the internal chamber of the housing.

The electrically powered negative pressure pump according to any preceding clause, further comprising a housing configured to house electronic controls configured to maintain the continuous negative pressure to the sleeve of the anchoring device.

The electrically powered negative pressure pump according to any preceding clause, wherein the expandable sealing mechanism comprises a low-profile balloon.

The electrically powered negative pressure pump according to any preceding clause, wherein the expandable sealing mechanism comprises two or more expandable sealing mechanisms.

The electrically powered negative pressure pump according to any preceding clause, wherein each of the two or more expandable sealing mechanisms comprises a low-profile balloon.

The electrically powered negative pressure pump according to any preceding clause, wherein the electrically powered negative pressure pump includes a long-lasting battery.

The electrically powered negative pressure pump according to any preceding clause, wherein the negative pressure brings the roughening material disposed on the outer surface of the sleeve into contact with the inner wall of the tissue cavity thereby creating frictional force that resists displacement of the sleeve.

A electrically powered negative pressure pump for use with a sleeve configured to be anchored within a tissue cavity of a patient, the electrically powered negative pressure pump comprising (i) a housing and (ii) a negative pressure indicator configured to detect when a negative pressure provided to the sleeve drops below a pre-determined threshold value, wherein the electrically powered negative pressure pump is configured to provide a continuous negative pressure to the sleeve in a range of from −50 mmHg to −150 mmHg.

The electrically powered negative pressure pump according to any preceding clause, further comprising at least one pressure tube configured to connect to the sleeve, to thereby provide negative pressure to the sleeve to create a frictional force that resists displacement of the sleeve from the tissue cavity.

The electrically powered negative pressure pump according to any preceding clause, wherein the at least one pressure tube is configured to connect to the sleeve via an one-way valve.

The electrically powered negative pressure pump according to any preceding clause, further comprising a controller configured to maintain the continuous negative pressure to the sleeve of the anchoring device.

The electrically powered negative pressure pump according to any preceding clause, further comprising a collection canister configured to collect fluid from an area around the tissue cavity in which the sleeve is anchored.

The electrically powered negative pressure pump according to any preceding clause, wherein the sleeve further includes an expandable sealing mechanism at a proximal end of the sleeve.

The electrically powered negative pressure pump according to any preceding clause, wherein the sleeve further includes an expandable sealing mechanism at a distal end of the sleeve.

The electrically powered negative pressure pump according to any preceding clause, wherein the sleeve further includes a roughening material along an outer surface of the sleeve.

The electrically powered negative pressure pump according to any preceding clause, wherein the roughening material comprises at least one of a porous fiber or matrix, a polymeric material having small protrusions, an open-cell foam, or a combination thereof.

The electrically powered negative pressure pump according to any preceding clause, further comprising one or more of (i) a full canister alarm, (ii) a leak alarm, and (iii) an idle alarm.

The electrically powered negative pressure pump according to any preceding clause, wherein the pre-determined threshold value is −70 mmHg.

The electrically powered negative pressure pump according to any preceding clause, wherein the pre-determined threshold value is −60 mmHg.

The electrically powered negative pressure pump according to any preceding clause, wherein the negative pressure indicator includes an alarm comprising an audible signal.

The electrically powered negative pressure pump according to any preceding clause, wherein the negative pressure indicator comprises a display screen.

The electrically powered negative pressure pump according to any preceding clause, wherein the collection canister comprises a housing that creates an internal chamber configured to hold the fluid collected from an area around the tissue cavity.

The electrically powered negative pressure pump according to any preceding clause, wherein the collection canister further comprises an inlet into which the fluid collected from an area around the tissue cavity enters the internal chamber of the housing.

The electrically powered negative pressure pump according to any preceding clause, further comprising a housing configured to house electronic controls configured to maintain the continuous negative pressure to the sleeve of the anchoring device.

The electrically powered negative pressure pump according to any preceding clause, wherein the expandable sealing mechanism comprises a low-profile balloon.

The electrically powered negative pressure pump according to any preceding clause, wherein the expandable sealing mechanism comprises two or more expandable sealing mechanisms.

The electrically powered negative pressure pump according to any preceding clause, wherein each of the two or more expandable sealing mechanisms comprises a low-profile balloon.

The electrically powered negative pressure pump according to any preceding clause, wherein the electrically powered negative pressure pump includes a long-lasting battery.

The electrically powered negative pressure pump according to any preceding clause, wherein the negative pressure brings the outer surface of the sleeve into contact with an inner wall of the tissue cavity thereby creating frictional force that resists displacement of the sleeve.

The electrically powered negative pressure pump according to any preceding clause, wherein the negative pressure brings the roughening material disposed on the outer surface of the sleeve into contact with an inner wall of the tissue cavity thereby creating frictional force that resists displacement of the sleeve.

An anchoring system comprising a sleeve having an inner surface defining an inner lumen, an expandable sealing mechanism disposed along at least one end of the sleeve, and a sheath covering the outer surface of the sleeve and the expandable sealing mechanism, wherein the sheath is disposed through the inner lumen of the sleeve and through an entire length of the anchoring system to a region of the anchoring system that is outside of a patient and at an opposite end of the inner lumen where the sleeve is disposed, such that the sheath everts back onto itself, and extends back down the length of the anchoring system on the outer surface of the sleeve, thereby covering the sleeve, wherein the sheath is configured to be removed via the inner lumen after insertion of the system into a tissue cavity.

The anchoring system according to any preceding clause, wherein, during placement of the sleeve within the tissue cavity, the sheath is doubled over itself in order to cover the outer surface of the sleeve having the expandable sealing mechanism and everts back over itself in order to extend back into the inner lumen of the sleeve.

The anchoring system according to any preceding clause, wherein the sheath is configured to be removed by applying tension on the sheath at an end of the sheath that is opposite to the sleeve thereby causing the sheath to de-evert, uncovering the outer surface of the sleeve, and allowing the sheath to be removed by pulling the sheath through the inner lumen.

The anchoring system according to any preceding clause, wherein the sheath is attached circumferentially at attachments points to inside of the inner lumen of the sleeve.

The anchoring system according to any preceding clause, wherein the sheath extends through the inner lumen of the sleeve to provide an enteric conduit for enteric matter flowing through the sleeve when in use.

The anchoring system according to any preceding clause, wherein expansion of the expandable sealing mechanism and application of negative pressure to the anchoring system, causes a seal to form between the expandable sealing mechanism, the outer surface of the sleeve, and an inner surface of the tissue cavity.

The anchoring system according to any preceding clause, wherein the sheath comprises a smooth material.

The anchoring system according to any preceding clause, wherein the sleeve further includes a roughening material along an outer surface of the sleeve.

The anchoring system according to any preceding clause, wherein the roughening material comprises at least one of a porous fiber or matrix, a polymeric material having small protrusions, an open-cell foam, or a combination thereof.

The anchoring system according to any preceding clause, wherein the expandable sealing mechanism comprises a low-profile balloon.

The anchoring system according to any preceding clause, wherein the expandable sealing mechanism is expanded by providing a non-compressible fluid to expand the expandable sealing mechanism.

The anchoring system according to any preceding clause, wherein the sleeve includes a first expandable sealing mechanism along a proximal end of the sleeve and a second expandable sealing mechanism along a distal end of the sleeve.

The anchoring system according to any preceding clause, wherein each of the first and second expandable sealing mechanisms comprises a low-profile balloon.

The anchoring system according to any preceding clause, wherein the first and second expandable sealing mechanisms are expanded by providing a non-compressible fluid to expand the first and second expandable sealing mechanisms.

The anchoring system according to any preceding clause, wherein the application of negative pressure brings the roughening material disposed on the outer surface of the sleeve into contact with the inner surface of the tissue cavity thereby creating frictional force that resists displacement of the sleeve.

The anchoring system according to any preceding clause, further comprising (i) a third expandable sealing mechanism disposed along the proximal end of the sleeve, and (ii) a fourth expandable sealing mechanism disposed along the distal end of the sleeve.

The anchoring system according to any preceding clause, further comprising an anchor portion that includes a first expandable sealing mechanism disposed along a proximal end of the sleeve, a second expandable sealing mechanism disposed along the proximal end of the sleeve and a roughening material positioned between the first and second expandable sealing mechanisms.

The anchoring system according to any preceding clause, wherein a lubricant or other friction-reducing substance is provided along one or more of (i) inner and/or outer surfaces of the sheath, (i) an inner surface of the inner lumen, and (iii) the outer surface of the sleeve.

The anchoring system according to any preceding clause, wherein the lubricant or other friction-reducing substance comprises at least one of (i) a liquid, (ii) a surface coating, (iii) a solid particulate, or (iv) a combination thereof.

The anchoring system according to any preceding clause, wherein the lubricant or other friction-reducing substance are pre-applied to the anchoring system during manufacturing and are already in place when a user is presented with the anchoring system for use.

The anchoring system according to any preceding clause, wherein the lubricant or other friction-reducing substance are provided with a packaging of the anchoring system and a user applies them to the anchoring system prior to use.

The anchoring system according to any preceding clause, wherein a first type of lubricant is provided between the outer surface of the sheath and the inner surface of the lumen and a second type of the lubricant, which is differ from the first type of lubricant, is provided between the inner surface of the sheath and the outer surface of the sleeve.

The anchoring system according to any preceding clause, wherein the anchoring system further includes a semi-rigid shaft element that is disposed within the inner lumen of the sleeve and couples to the anchoring system, such that it may be used to advance the anchoring system to its desired position.

The anchoring system according to any preceding clause, wherein the semi-rigid shaft element attaches to at least one of an endoscope, a colonoscope, or a sigmoidoscope, upon which the shaft element is able to advance the anchoring system into a patient.

The anchoring system according to any preceding clause, wherein the sheath is coupled to a hollow cylindrical slider element, with this slider element disposed within a cylindrical handle.

The anchoring system according to any preceding clause, wherein the cylindrical handle further comprises one or more cutouts disposed axially, wherein a portion of the slider element may protrude through the one or more cutouts.

The anchoring system according to any preceding clause, wherein the sheath and slider element are joined mechanically together such that they create one continuous luminal path to the opposite end of the anchoring system.

The anchoring system according to any preceding clause, further comprising a negative pressure source.

The anchoring system according to any preceding clause, wherein the negative pressure source comprises an electrically powered negative pressure pump attached to a collection canister.

The anchoring system according to any preceding clause, wherein the electrically powered negative pressure pump is configured to provide a continuous negative pressure in a range from around −50 mmHg to −150 mmHg.

The anchoring system according to any preceding clause, wherein the electrically powered negative pressure pump includes a low pressure indicator or alarm.

The anchoring system according to any preceding clause, wherein the electrically powered negative pressure pump includes one or more of (i) a full canister alarm, (ii) a low pressure alarm, (iii) a leak alarm, and (iv) an idle alarm.

The anchoring system according to any preceding clause, wherein the electrically powered negative pressure pump includes a long-lasting battery.

The anchoring system according to any preceding clause, wherein the first and second expandable sealing mechanisms are expanded to create a difference in luminal occupancy that results in a step off between the first and second expandable sealing mechanisms and the inner lumen in an area in which a seal is formed between the first and second expandable sealing mechanisms, the outer surface of the sleeve, and the inner surface of the tissue cavity when negative pressure is applied, such that the inner surface of the tissue cavity conforms to this step off, and thereby displacement forces required to slide the sleeve within the tissue cavity are increased.

An anchoring system comprising a sleeve having an inner surface defining an inner lumen, a roughening material along an outer surface of the sleeve, an expandable sealing mechanism disposed along at least one end of the sleeve, and a sheath covering the outer surface of the sleeve, the roughening material, and the expandable sealing mechanism in order to reduce friction created by the roughening material when being inserted into a tissue cavity, wherein the sheath is disposed through the inner lumen of the sleeve and through an entire length of the anchoring system to a region of the anchoring system that is outside of a patient and at an opposite end of the inner lumen where the sleeve is disposed, such that the sheath everts back onto itself, and extends back down the length of the anchoring system on the outer surface of the sleeve, thereby covering the sleeve, the roughening material, and the expandable sealing mechanism, wherein the sheath is configured to be removed via the inner lumen after insertion of the system into the tissue cavity.

The anchoring system according to any preceding clause, wherein, during placement of the sleeve within the tissue cavity, the sheath is doubled over itself in order to cover the outer surface of the sleeve having the roughening material and the expandable sealing mechanism and everts back over itself in order to extend back into the inner lumen of the sleeve.

The anchoring system according to any preceding clause, wherein the sheath is configured to be removed by applying tension on the sheath at an end of the sheath that is opposite to the sleeve thereby causing the sheath to de-evert, uncovering the outer surface of the sleeve, the roughening material, and the expandable sealing mechanism, and allowing the sheath to be removed by pulling the sheath through the inner lumen.

The anchoring system according to any preceding clause, wherein the sheath is attached circumferentially at attachments points to inside of the inner lumen of the sleeve.

The anchoring system according to any preceding clause, wherein the sheath extends through the inner lumen of the sleeve to provide an enteric conduit for enteric matter flowing through the sleeve when in use.

The anchoring system according to any preceding clause, wherein expansion of the expandable sealing mechanism and application of negative pressure to the anchoring system, causes a seal to form between the expandable sealing mechanism, the outer surface of the sleeve, and an inner surface of the tissue cavity.

The anchoring system according to any preceding clause, wherein the sheath comprises a smooth material.

The anchoring system according to any preceding clause, wherein the roughening material comprises at least one of a porous fiber or matrix, a polymeric material having small protrusions, an open-cell foam, or a combination thereof.

The anchoring system according to any preceding clause, wherein the expandable sealing mechanism comprises a low-profile balloon.

The anchoring system according to any preceding clause, wherein the expandable sealing mechanism is expanded by providing a non-compressible fluid to expand the expandable sealing mechanism.

The anchoring system according to any preceding clause, wherein the sleeve includes a first expandable sealing mechanism along a proximal end of the sleeve and a second expandable sealing mechanism along a distal end of the sleeve.

The anchoring system according to any preceding clause, wherein each of the first and second expandable sealing mechanisms comprises a low-profile balloon.

The anchoring system according to any preceding clause, wherein the first and second expandable sealing mechanisms are expanded by providing a non-compressible fluid to expand the first and second expandable sealing mechanisms.

The anchoring system according to any preceding clause, wherein the application of negative pressure brings the roughening material disposed on the outer surface of the sleeve into contact with the inner surface of the tissue cavity thereby creating frictional force that resists displacement of the sleeve.

The anchoring system according to any preceding clause, further comprising (i) a third expandable sealing mechanism disposed along the proximal end of the sleeve, and (ii) a fourth expandable sealing mechanism disposed along the distal end of the sleeve.

The anchoring system according to any preceding clause, further comprising an anchor portion that includes a first expandable sealing mechanism disposed along a proximal end of the sleeve, a second expandable sealing mechanism disposed along the proximal end of the sleeve and the roughening material positioned between the first and second expandable sealing mechanisms.

The anchoring system according to any preceding clause, wherein a lubricant or other friction-reducing substance is provided along one or more of (i) inner and/or outer surfaces of the sheath, (i) an inner surface of the inner lumen, and (iii) the outer surface of the sleeve.

The anchoring system according to any preceding clause, wherein the lubricant or other friction-reducing substance comprises at least one of (i) a liquid, (ii) a surface coating, (iii) a solid particulate, or (iv) a combination thereof.

The anchoring system according to any preceding clause, wherein the lubricant or other friction-reducing substance are pre-applied to the anchoring system during manufacturing and are already in place when a user is presented with the anchoring system for use.

The anchoring system according to any preceding clause, wherein the lubricant or other friction-reducing substance are provided with a packaging of the anchoring system and a user applies them to the anchoring system prior to use.

The anchoring system according to any preceding clause, wherein a first type of lubricant is provided between the outer surface of the sheath and the inner surface of the lumen and a second type of the lubricant, which is differ from the first type of lubricant, is provided between the inner surface of the sheath and the outer surface of the sleeve.

The anchoring system according to any preceding clause, wherein the anchoring system further includes a semi-rigid shaft element that is disposed within the inner lumen of the sleeve and couples to the anchoring system, such that it may be used to advance the anchoring system to its desired position.

The anchoring system according to any preceding clause, wherein the semi-rigid shaft element attaches to at least one of an endoscope, a colonoscope, or a sigmoidoscope, upon which the shaft element is able to advance the anchoring system into a patient.

The anchoring system according to any preceding clause, wherein the sheath is coupled to a hollow cylindrical slider element, with this slider element disposed within a cylindrical handle.

The anchoring system according to any preceding clause, wherein the cylindrical handle further comprises one or more cutouts disposed axially, wherein a portion of the slider element may protrude through the one or more cutouts.

The anchoring system according to any preceding clause, wherein the sheath and slider element are joined mechanically together such that they create one continuous luminal path to the opposite end of the anchoring system.

The anchoring system according to any preceding clause, further comprising a negative pressure source.

The anchoring system according to any preceding clause, wherein the negative pressure source comprises an electrically powered negative pressure pump attached to a collection canister.

The anchoring system according to any preceding clause, wherein the electrically powered negative pressure pump is configured to provide a continuous negative pressure in a range from around −50 mmHg to −150 mmHg.

The anchoring system according to any preceding clause, wherein the electrically powered negative pressure pump includes a low pressure indicator or alarm.

The anchoring system according to any preceding clause, wherein the electrically powered negative pressure pump includes one or more of (i) a full canister alarm, (ii) a low pressure alarm, (iii) a leak alarm, and (iv) an idle alarm.

The anchoring system according to any preceding clause, wherein the electrically powered negative pressure pump includes a long-lasting battery.

The anchoring system according to any preceding clause, wherein the first and second expandable sealing mechanisms are expanded to create a difference in luminal occupancy that results in a step off between the first and second expandable sealing mechanisms and the inner lumen in an area in which a seal is formed between the first and second expandable sealing mechanisms, the outer surface of the sleeve, and the inner surface of the tissue cavity when negative pressure is applied, such that the inner surface of the tissue cavity conforms to this step off, and thereby displacement forces required to slide the sleeve within the tissue cavity are increased.

A sleeve configured to be anchored within a tissue cavity of a patient, the sleeve having an outer surface for contacting an inner wall of the tissue cavity and an expandable sealing mechanism for isolating a portion of the tissue cavity adjacent to the sleeve from a remainder of the tissue cavity, wherein the expandable sealing mechanism comprises a low-profile balloon having a flap configured to attach to the sleeve.

The sleeve according to any preceding clause, wherein the low-profile balloon has a flattened top.

The sleeve according to any preceding clause, wherein the low-profile balloon has a varying thickness.

The sleeve according to any preceding clause, wherein an area of the flap of the low-profile balloon has a thickness that is greater than a thickness of the rest of the balloon.

The sleeve according to any preceding clause, wherein the area of the flap of the low-profile balloon is 20% to 30% thicker than a thickness of the rest of the balloon.

The sleeve according to any preceding clause, wherein the sleeve includes an inner lumen.

The sleeve according to any preceding clause, wherein a sheath extends through the inner lumen of the sleeve to provide an enteric conduit for enteric matter flowing through the sleeve when in use.

The sleeve according to any preceding clause, wherein expansion of the expandable sealing mechanism and application of negative pressure to the sleeve, causes a seal to form between the expandable sealing mechanism, the outer surface of the sleeve, and an inner surface of the tissue cavity.

The sleeve according to any preceding clause, wherein the sheath comprises a smooth material.

The sleeve according to any preceding clause, wherein the sleeve further includes a roughening material along the outer surface of the sleeve.

The sleeve according to any preceding clause, wherein the roughening material comprises at least one of a porous fiber or matrix, a polymeric material having small protrusions, an open-cell foam.

The sleeve according to any preceding clause, wherein the expandable sealing mechanism is expanded by providing a non-compressible fluid to expand the expandable sealing mechanism.

The sleeve according to any preceding clause, wherein the sleeve includes a first expandable sealing mechanism along a proximal end of the sleeve and a second expandable sealing mechanism along a distal end of the sleeve.

The sleeve according to any preceding clause, wherein each of the first and second expandable sealing mechanisms comprises a low-profile balloon.

The sleeve according to any preceding clause, wherein the first and second expandable sealing mechanisms are expanded by providing a non-compressible fluid to expand the first and second expandable sealing mechanisms.

The sleeve according to any preceding clause, wherein the application of negative pressure brings the outer surface of the sleeve into contact with the inner surface of the tissue cavity thereby creating frictional force that resists displacement of the sleeve.

The sleeve according to any preceding clause, wherein the application of negative pressure brings the roughening material disposed along the outer surface of the sleeve into contact with the inner surface of the tissue cavity thereby creating frictional force that resists displacement of the sleeve.

The sleeve according to any preceding clause, further comprising (i) a third expandable sealing mechanism disposed along the proximal end of the sleeve, and (ii) a fourth expandable sealing mechanism disposed along the distal end of the sleeve.

The sleeve according to any preceding clause, further comprising an anchor portion that includes a first expandable sealing mechanism disposed along a proximal end of the sleeve, a second expandable sealing mechanism disposed along the proximal end of the sleeve and the roughening material positioned between the first and second expandable sealing mechanisms.

The sleeve according to any preceding clause, wherein a lubricant or other friction-reducing substance is provided along one or more of (i) inner and/or outer surfaces of the sheath, (i) an inner surface of the inner lumen, and (iii) the outer surface of the sleeve.

The sleeve according to any preceding clause, wherein the lubricant or other friction-reducing substance comprises at least one of (i) a liquid, (ii) a surface coating, (iii) a solid particulate, or (iv) a combination thereof.

The sleeve according to any preceding clause, wherein the lubricant or other friction-reducing substance are pre-applied to the sleeve during manufacturing and are already in place when a user is presented with the sleeve for use.

The sleeve according to any preceding clause, wherein the lubricant or other friction-reducing substance are provided with a packaging of the sleeve and a user applies them to the sleeve prior to use.

The sleeve according to any preceding clause, wherein a first type of lubricant is provided between the outer surface of the sheath and the inner surface of the lumen and a second type of the lubricant, which is differ from the first type of lubricant, is provided between the inner surface of the sheath and the outer surface of the sleeve.

The sleeve according to any preceding clause, wherein the sleeve further includes a semi-rigid shaft element that is disposed within the inner lumen of the sleeve and couples to the sleeve, such that it may be used to advance the sleeve to its desired position.

The sleeve according to any preceding clause, wherein the semi-rigid shaft element attaches to at least one of an endoscope, a colonoscope, or a sigmoidoscope, upon which the shaft element is able to advance the sleeve into a patient.

The sleeve according to any preceding clause, wherein the sheath is coupled to a hollow cylindrical slider element, with this slider element disposed within a cylindrical handle.

The sleeve according to any preceding clause, wherein the cylindrical handle further comprises one or more cutouts disposed axially, wherein a portion of the slider element may protrude through the one or more cutouts.

The sleeve according to any preceding clause, wherein the sheath and slider element are joined mechanically together such that they create one continuous luminal path to the opposite end of the sleeve.

The sleeve according to any preceding clause, further comprising a negative pressure source.

The sleeve according to any preceding clause, wherein the negative pressure source comprises an electrically powered negative pressure pump attached to a collection canister.

The sleeve according to any preceding clause, wherein the electrically powered negative pressure pump is configured to provide a continuous negative pressure in a range from around −50 mmHg to −150 mmHg.

The sleeve according to any preceding clause, wherein the electrically powered negative pressure pump includes a low pressure indicator or alarm.

The sleeve according to any preceding clause, wherein the electrically powered negative pressure pump includes one or more of (i) a full canister alarm, (ii) a low pressure alarm, (iii) a leak alarm, and (iv) an idle alarm.

The sleeve according to any preceding clause, wherein the electrically powered negative pressure pump includes a long-lasting battery.

The sleeve according to any preceding clause, wherein the first and second expandable sealing mechanisms are expanded to create a difference in luminal occupancy that results in a step off between the first and second expandable sealing mechanisms and the inner lumen in an area in which a seal is formed between the first and second expandable sealing mechanisms, the outer surface of the sleeve, and the inner surface of the tissue cavity when negative pressure is applied, such that the inner surface of the tissue cavity conforms to this step off, and thereby displacement forces required to slide the sleeve within the tissue cavity are increased.

An anchoring system comprising a sleeve having an inner surface defining an inner lumen, a first expandable sealing mechanism disposed along a proximal end of the sleeve, a second expandable sealing mechanism disposed along the proximal end of the sleeve, and a negative pressure system configured to provide negative pressure to the sleeve to cause the sleeve to remain in place in a tissue cavity, wherein expansion of the first and second expandable sealing mechanisms and application of negative pressure to the sleeve via the negative pressure system, creates a bypass for fluid, gas and/or solid matter flowing through the inner lumen of the sleeve such that a bolus of fluid, gas and/or solid matter has unrestricted flow down the sleeve to prevent build up of pressure or expansile forces in the bowel.

The anchoring system according to the preceding clause, wherein the sleeve further comprises a roughening material disposed on an outer surface of the sleeve.

The anchoring system according to any preceding clause, wherein the roughening material comprises at least one of a porous fiber or matrix, a polymeric material having small protrusions, an open-cell foam, or a combination thereof.

The anchoring system according to any preceding clause, wherein the application of negative pressure brings the roughening material disposed on the outer surface of the sleeve into contact with an inner surface of the tissue cavity thereby creating frictional force that resists displacement of the sleeve.

The anchoring system according to any preceding clause, wherein each of the first and second expandable sealing mechanisms comprises a low-profile balloon.

The anchoring system according to any preceding clause, wherein the first and second expandable sealing mechanisms are expanded by providing a non-compressible to inflate or expand the first and second expandable sealing mechanisms.

The anchoring system according to any preceding clause, wherein the negative pressure brings an outer surface of the sleeve into contact with the inner wall of the tissue cavity thereby creating frictional force that resists displacement of the sleeve.

The anchoring system according to any preceding clause, further comprising (i) a third expandable sealing mechanism disposed along the proximal end of the sleeve, and (ii) a fourth expandable sealing mechanism disposed along the distal end of the sleeve.

The anchoring system according to any preceding clause, further comprising an anchor portion that includes the first expandable sealing mechanism disposed along the proximal end of the sleeve, the second expandable sealing mechanism disposed along the distal end of the sleeve, and a roughening material positioned between the first and second expandable sealing mechanisms.

The anchoring system according to any preceding clause, further comprising a sheath covering an outer surface of the sleeve and the first and second expandable sealing mechanisms.

The anchoring system according to any preceding clause, wherein the sheath is configured to be removed via the inner lumen after insertion of the system into a tissue cavity.

The anchoring system according to any preceding clause, wherein a lubricant or other friction-reducing substance is provided along one or more of (i) inner and/or outer surfaces of the sheath, (i) an inner surface of the inner lumen, and (iii) the outer surface of the sleeve.

The anchoring system according to any preceding clause, wherein the lubricant or other friction-reducing substance comprises at least one of (i) a liquid, (ii) a surface coating, (iii) a solid particulate, or (iv) a combination thereof.

The anchoring system according to any preceding clause, wherein the lubricant or other friction-reducing substance are pre-applied to the anchoring system during manufacturing and are already in place when a user is presented with the anchoring system for use.

The anchoring system according to any preceding clause, wherein the lubricant or other friction-reducing substance are provided with a packaging of the anchoring system and a user applies them to the anchoring system prior to use.

The anchoring system according to any preceding clause, wherein a first type of lubricant is provided between the outer surface of the sheath and the inner surface of the lumen and a second type of the lubricant, which is differ from the first type of lubricant, is provided between the inner surface of the sheath and the outer surface of the sleeve.

The anchoring system according to any preceding clause, wherein the anchoring system further includes a semi-rigid shaft element that is disposed within the inner lumen of the sleeve and couples to the anchoring system, such that it may be used to advance the anchoring system to its desired position.

The anchoring system according to any preceding clause, wherein the semi-rigid shaft element attaches to at least one of an endoscope, a colonoscope, or a sigmoidoscope, upon which the shaft element is able to advance the anchoring system into a patient.

The anchoring system according to any preceding clause, wherein the sheath is coupled to a hollow cylindrical slider element, with this slider element disposed within a cylindrical handle.

The anchoring system according to any preceding clause, wherein the cylindrical handle further comprises one or more cutouts disposed axially, wherein a portion of the slider element may protrude through the one or more cutouts.

The anchoring system according to any preceding clause, wherein the sheath and slider element are joined mechanically together such that they create one continuous luminal path to the opposite end of the anchoring system.

The anchoring system according to any preceding clause, wherein the negative pressure system comprises an electrically powered negative pressure pump.

The anchoring system according to any preceding clause, wherein the electrically powered negative pressure pump is attached to a collection canister.

The anchoring system according to any preceding clause, wherein the electrically powered negative pressure pump is configured to provide a continuous negative pressure in a range from around −50 mmHg to −150 mmHg.

The anchoring system according to any preceding clause, wherein the electrically powered negative pressure pump includes a low pressure indicator or alarm.

The anchoring system according to any preceding clause, wherein the electrically powered negative pressure pump includes one or more of (i) a full canister alarm, (ii) a low pressure alarm, (iii) a leak alarm, and (iv) an idle alarm.

The anchoring system according to any preceding clause, wherein the electrically powered negative pressure pump includes a long-lasting battery.

The anchoring system according to any preceding clause, wherein the first and second expandable sealing mechanisms are expanded to create a difference in luminal occupancy that results in a step off between the first and second expandable sealing mechanisms and the inner lumen in an area in which a seal is formed between the first and second expandable sealing mechanisms, the outer surface of the sleeve, and the inner surface of the tissue cavity when negative pressure is applied, such that the inner surface of the tissue cavity conforms to this step off, and thereby displacement forces required to slide the sleeve within the tissue cavity are increased.

An anchoring system comprising a sleeve having an inner surface defining an inner lumen, a first expandable sealing mechanism disposed along a proximal end of the sleeve, a second expandable sealing mechanism disposed along the proximal end of the sleeve, a roughening material disposed on an outer surface of the sleeve, and a sheath configured to cover the outer surface of the sleeve, the roughening material, and the first and second expandable sealing mechanisms, wherein the sheath is configured to be removed via the inner lumen after insertion of the system into a tissue cavity, wherein the anchoring system comprises (i) a first configuration in which the sheath covers the outer surface of the sleeve, the roughening material, and the first and second expandable sealing mechanisms, and (ii) a second configuration in which the sheath is removed from the system after insertion of the system into a tissue cavity, and wherein expansion of the first and second expandable sealing mechanisms and application of negative pressure to the anchoring system, causes a seal to form between the first and second expandable sealing mechanisms, the outer surface of the sleeve, and an inner surface of a tissue cavity.

1. An anchoring system comprising:
a sleeve having an inner surface defining an inner lumen;
a first inflatable sealing mechanism disposed n the sleeve, the first inflatable sealing mechanism comprising a low-profile balloon;
a second inflatable sealing mechanism disposed on the sleeve, the second inflatable sealing mechanism comprising a low-profile balloon;
a roughening material disposed on an outer surface of the sleeve and positioned between the first inflatable sealing mechanism and the second inflatable sealing mechanism; and
a sheath configured to cover at least one of the outer surface of the sleeve, the roughening material, or the first and second inflatable sealing mechanisms,
wherein the sheath is configured to be removed via the inner lumen after insertion of the system into a tissue cavity, and
wherein expansion of the first and second inflatable sealing mechanisms and application of negative pressure to the anchoring system, causes a seal to form between the first and second inflatable sealing mechanisms, the outer surface of the sleeve, and an inner surface of a tissue cavity.

2. The anchoring system according to claim 1, wherein the roughening material comprises at least one of a porous fiber or matrix, a polymeric material having small protrusions, an open-cell foam, or a combination thereof.

3. The anchoring system according to claim 1, wherein the application of negative pressure brings the roughening material disposed on the outer surface of the sleeve into contact with the inner surface of the tissue cavity thereby creating frictional force that resists displacement of the sleeve.

4. The anchoring system according to claim 1, wherein the first and second inflatable sealing mechanisms are expanded by providing a non-compressible fluid to inflate or expand the first and second inflatable sealing mechanisms.

5. (canceled)

6. The anchoring system according to claim 1, wherein the sheath is disposed through the inner lumen and through an entire length of the anchoring system to a region of the anchoring system that is outside of a patient and at an opposite end of the inner lumen where an anchor portion is disposed, such that the sheath everts back onto itself, and extends back down the length of the anchoring system on the outer surface, thereby covering the anchor portion.

7. The anchoring system according to claim 6, wherein the anchor portion includes the first inflatable sealing mechanism, the second inflatable sealing mechanism, and the roughening material positioned between the first and second inflatable sealing mechanisms.

8. The anchoring system according to claim 7, wherein the sheath is configured to be removed by applying tension on the sheath at an end of the sheath that is opposite to the anchor portion thereby causing the sheath to de-evert, uncovering the anchor portion, and allowing the sheath to be removed by pulling the sheath through the inner lumen.

9. The anchoring system according to claim 1, wherein a lubricant or other friction-reducing substance is provided along one or more of (i) inner and/or outer surfaces of the sheath, (i) an inner surface of the inner lumen, and (iii) the outer surface of the sleeve.

10. The anchoring system according to claim 9, wherein the lubricant or other friction-reducing substance comprises at least one of (i) a liquid, (ii) a surface coating, (iii) a solid particulate, or (iv) a combination thereof.

11. The anchoring system according to claim 9, wherein the lubricant or other friction-reducing substance are one of (i) pre-applied to the anchoring system during manufacturing and are already in place when a user is presented with the anchoring system for use, or (ii) provided with a packaging of the anchoring system and a user applies them to the anchoring system prior to use.

12. The anchoring system according to claim 1, wherein the anchoring system further includes a semi-rigid shaft element that is disposed within the inner lumen of the sleeve and couples to the anchoring system, such that it may be used to advance the anchoring system to its desired position.

13. The anchoring system according to claim 12, wherein the semi-rigid shaft element attaches to at least one of an endoscope, a colonoscope, or a sigmoidoscope, upon which the shaft element is able to advance the anchoring system into a patient.

14. The anchoring system according to claim 1, wherein the sheath is coupled to a hollow cylindrical slider element, with this slider element disposed within a cylindrical handle.

15. The anchoring system according to claim 14, wherein the cylindrical handle further comprises one or more cutouts disposed axially, wherein a portion of the slider element may protrude through the one or more cutouts.

16. The anchoring system according to claim 14, wherein the sheath and slider element are joined mechanically together such that they create one continuous luminal path to the opposite end of the anchoring system.

17. The anchoring system according to claim 1, further comprising a negative pressure source.

18. The anchoring system according to claim 17, wherein the negative pressure source comprises an electrically powered negative pressure pump attached to a collection canister.

19. The anchoring system according to claim 18, wherein the electrically powered negative pressure pump is configured to provide a continuous negative pressure in a range from around −50 mmHg to −150 mmHg.

20. The anchoring system according to claim 18, wherein the electrically powered negative pressure pump includes a low pressure indicator or alarm.

21. The anchoring system according to claim 18, wherein the electrically powered negative pressure pump includes one or more of (i) a full canister alarm, (ii) a low pressure alarm, (iii) a leak alarm, and (iv) an idle alarm.

22. The anchoring system according to claim 18, wherein the electrically powered negative pressure pump includes a long-lasting battery.

23. The anchoring system according to claim 1, wherein the first and second inflatable sealing mechanisms are expanded to create a difference in luminal occupancy that results in a step off between the first and second inflatable sealing mechanisms and the inner lumen in an area in which the seal is formed between the first and second inflatable sealing mechanisms, the outer surface of the sleeve, and the inner surface of the tissue cavity when negative pressure is applied, such that the inner surface of the tissue cavity conforms to this step off, and thereby displacement forces required to slide the sleeve within the tissue cavity are increased.

24. A method for anchoring a sleeve of an anchoring system in a tissue cavity, with the sleeve having an outer surface comprising at least two expandable sealing mechanisms for isolating a portion of the tissue cavity adjacent to the sleeve from a remainder of the tissue cavity, the method comprising:
inserting the sleeve of the anchoring system of claim 1 in the tissue cavity;
expanding the first and second expandable sealing mechanisms to create a seal between the first and second expandable sealing mechanisms and an inner surface of a tissue cavity; and
applying a constant negative pressure in a range of −50 mmHg to −150 mmHg using an electrically powered negative pressure pump to a region between an outer surface of the sleeve and an inner surface of the isolated portion of the tissue cavity to create a frictional force between the outer surface of the sleeve and the inner surface of the tissue cavity.

25. The method according to claim 24, wherein the step of expanding the first and second expandable sealing mechanisms is conducted by injecting a non-compressible liquid into each of the first and second expandable sealing mechanisms.

26. (canceled)

27. The method according to claim 24, wherein the step of inserting the sleeve in the tissue cavity is conducted using a delivery system that includes a semi-rigid shaft element.

28. The method according to claim 24, wherein the method further includes removing the sleeve from the tissue cavity by (i) releasing the negative pressure, (ii) collapsing the first and second expandable sealing mechanisms, and (iii) injecting an amount of saline through an irrigation tubing to break the seal.

29. An anchoring device configured to be anchored within a bowel of a patient, the anchoring device comprising:
a sleeve configured to be positioned within the bowel of the patient;
an external portion configured to extend externally from the bowel of the patient; and
two redundant methods of anchoring the device within the bowel,
wherein a first method of anchoring the device utilizes a negative pressure-based system that comprises an electrically powered negative pressure pump that applies negative pressure to the device to create a frictional force that resists displacement of the sleeve of the device from the bowel,
wherein a second method of anchoring the device utilizes first and second expandable sealing mechanisms, wherein each of the first and second expandable sealing mechanisms comprises a low-profile balloon wherein expansion of the first and second expandable sealing mechanisms causes a seal to form between the first and second expandable sealing mechanisms and an inner surface of a tissue cavity,
wherein at least one of (i) a column strength of the sleeve is high enough to hold the device in place even if there is failure of the first method of anchoring, (ii) the first method of anchoring is sufficient to hold the device in place even if there is failure of the second method of anchoring, or (iii) the second method of anchoring is sufficient to hold the device in place even if there is failure of the first method of anchoring.

30. The anchoring system according to claim 1, wherein the anchoring system comprises (i) a first configuration in which the sheath covers at least one of the outer surface of the sleeve, the roughening material, or the first and second inflatable sealing mechanisms, and (ii) a second configuration in which the sheath is removed from the system after insertion of the system into a tissue cavity.
